(19) United States
(12) Patent Application Publication
(10) Pub. No.: US 2025/0127964 A1
(43) Pub. Date: Apr. 24, 2025

(54) SYSTEMS AND METHODS FOR GEL-BASED NEUROMODULATION

(71) Applicant: Tulavi Therapeutics, Inc., Los Gatos, CA (US)

(72) Inventor: Corinne Bright, Mountain View, CA (US)

(21) Appl. No.: 19/002,485

(22) Filed: Dec. 26, 2024

Related U.S. Application Data
(63) Continuation of application No. 16/981,199, filed on Sep. 15, 2020.
(60) Provisional application No. 62/643,174, filed on Mar. 15, 2018.

(51) Int. Cl. A61L 27/54; A61L 27/18; A61L 27/52; A61N 7/00; A61N 7/02; C08L 71/02

(52) U.S. Cl. CPC A61L 27/54; A61L 27/18; A61L 27/52; A61N 7/02; C08L 71/02; A61N 2007/003; C08L 2203/02

(57) ABSTRACT

Methods, devices and systems are described for gel-based modulation of neural tissue, including prevention of nerve regeneration and neuroma formation. The gel can be delivered to selected target locations within or proximate nerves, including interfascicularly and intrafascicularly. Gel delivery associated with an operative procedure for the treatment of pain and other indications is also disclosed.

### INCORPORATION BY REFERENCE TO ANY PRIORITY APPLICATIONS

This application is a continuation of U.S. application Ser. No. 16/981,199 filed on Sep. 15, 2020, which is a national stage under 35 U.S.C. § 371 of International Application No. PCT/US2019/022616, filed on Mar. 15, 2019, which claims the benefit under 35 U.S.C. § 119 (e) as a nonprovisional application of U.S. Provisional Application No. 62/643,174 filed on Mar. 15, 2018, each of which is hereby incorporated by reference in its entirety. Furthermore, any and all applications for which a foreign or domestic priority claim is identified in the Application Data Sheet as filed with the present application are hereby incorporated by reference under 37 CFR 1.57.

### BACKGROUND

### Field of the Invention

The invention relates in some aspects to systems and methods for neuromodulation utilizing including hydrogels, sympathetic neuromodulation, parasympathetic neuromodulation, central nervous system and peripheral somatic neuromodulation, including sensory, and motor nerve modulation. In particular, the development of in situ forming injectable nerve barriers comprised of synthetic polymers is disclosed. Numerous neurally-mediated diseases can be treated using a variety of neuromodulation techniques, including biomaterial based therapy, drug delivery based therapy, energy-based nerve therapy alone or in combination with existing neuromodulation techniques such as nerve blocks, nerve ablation, or neurolysis. Improved neuromodulation techniques with greater efficacy and/or decreased side effects are needed. In particular, methods to reduce the acute, subacute and chronic pain in patients are described. In delivering hydrogels loaded with various agents, a greater reduction and longer term pain relief can be realized. Furthermore, by delivering a neuroablative therapy (chemical, thermal etc) prior to or in conjunction with a non-growth permissive hydrogel at the target nerve, the efficacy of existing neuroablative therapies can be significantly extended as the nerves face an insurmountable barrier through which they can not regenerate to reach their target tissue.

### Description of the Related Art

Neuromodulation treatment options for reducing pain have expanded over the last 10 years. Long-established techniques for locally blocking nerves with anesthetic agents have expanded to include several new products that provide sustained release of anesthetic agents such a bupivacaine (Pacira Pharmaceuticals Exparel, DepoFoam particles) or bupivacaine and meloxicam (Heron Therapeutics HTX-011, particles). Both of these formulations provide sustained release of anesthetic when delivered locally to the surgical site, however the benefits in pain reduction to patients are measured in hours, not weeks. These formulations are delivered in particles to the surgical site where there are many fine nerve endings and smaller caliber nerves. The products also, by virtue of the fact that they do not target the larger caliber nerves, require the delivery of high volumes of the product in combination with additional bupivacaine, to achieve significant therapeutic benefit.

Long-established techniques for locally ablating nerves include the delivery of chemical agents such as ethanol and phenol, primarily to sensory nerves so there is not off target damage to motor nerves. In the periphery, while recognized to be efficacious, these are not routinely performed due to concerns about local toxicity and necrosis at the injection site. Similarly, there has been a resurgence of companies developing next-generation radiofrequency ablation systems, such as Avanos COOLIEF, to locally ablate nerves. All of these techniques, however, due to the strong regenerative propensity of nerves after injury, suffer from variable and poor long-term efficacy as the nerve eventually regenerate and/or form painful neuromas. In addition, the neuroablative drugs that are delivered rapidly spread and dissipate from the site and so the neuroablative effect is short lived and incomplete. Similarly, the lesion created by radiofrequency ablation is confined and so if improper technique is used or, as is often the case, the target nerve can not be identified under imaging, the thermal ablation efficacy is often poor due to incomplete ablation of the nerve.

Described herein are improvements on the existing therapeutic modalities utilizing in situ forming hydrogels to deliver the therapy directly to the nerves and retain it at the target site to reduce patient pain, improve their mobility and quality of life. The hydrogels may be delivered locally to the appropriate nerves to treat pain in patients with arthritis (e.g. osteoarthritis pain), pain from a neuroma (e.g. Morton's neuroma, neuroma after amputation, terminal neuroma or neuroma formed in continuity), after a traumatic injury, in the setting of treating a chronic pain patient, in preparation for a surgery (e.g. weeks to days prior to surgery), in lieu of a surgery as a way to provide another treatment option for patients that are not eligible or not ready to advance to a surgical solution for their pain, at the time of surgery, or post-operatively.

Of particular interest is leveraging the increasingly adopted use of ultrasound to deliver the therapy percutaneously to the nerves that are either the source of pain or innervate the distal source of pain. In the case of knee pain, hydrogels containing nerve blocking agents can be delivered to mixed nerves (sensory and motor) or sensory nerves that innervate the knee and surrounding soft tissue, including but not limited to, the femoral, sciatic, popliteal, and genicular nerves. In addition, the hydrogel would be suitable to minimize the high volumes of the particle based anesthetic blocks delivered through infiltration to the region. Examples include the delivery of sustained release of nerve blocking agents to the posterior capsule/fat pads or iPACK block. Nerve blocking agents can be incorporated into the hydrogels directly or encapsulated within micelles/nanoparticles which are then trapped in the hydrogel when it is formed in situ.

For treatments in which a permanent relief of pain is desired, such as patients who suffer from chronic pain, the in situ forming hydrogel can be delivered either alone or in conjunction with one of the existing therapeutic modalities (e.g. after the local delivery of a neurolytic agent such as capsaicin, RTX, ethanol, phenol or after the delivery of RF therapy). If the hydrogel is delivered alone, a nerve blocking or nerve ablation agent can be incorporated. The conventional neurolytic agents cause significant pain after injection as they are excitotoxic to the cells. Of particularly interest in the delivery of a hydrogel containing non excitotoxic agents at high enough concentrations to both reduce or block neurotransmitter and ATP release but also ablate nerves without significant pain. This permits the delivery of this therapy in a variety of clinical settings including in the clinic, in the ambulatory surgical center, or the hospital. It also permits a reduction in the expenses associated with the procedure, allowing for the use of blind and/or ultrasound guided injections performed under local anesthesia only with the patient awake.

Also of interest is the ability to combine the therapy with a surgical procedure to reduce costs associated with the treatment. The delivery of the hydrogel typically takes less than 5 minutes, including the time required to place the needle and then less than 20 seconds to deliver the hydrogel and thus there is not a significant impact to overall procedure time for procedures such as total knee arthroplasty or hip replacement. In these settings, the hydrogel can be delivered in a posterior/capsule/fat pad infiltration, iPACK, or genicular nerve block either prior to the surgery or immediately afterwards so that when a patient recovers they are already benefitting from the therapy.

### SUMMARY

Neuromodulatory hydrogels, with or without the addition of a neuromodulatory agent, are delivered to the desired neural target. Of particular interest to this disclosure is targeting peripheral nerve locations including locations with mixed nerves (motor, sensory, autonomic), mixed nerves (motor, sensory), or purely sensory or motor nerve bundles.

### Target Locations.

In some embodiments, gels can be delivered in association with an operative procedure of a joint. The operative procedure could be, for example, a spinal (e.g., cervical, thoracic, lumbar, and/or sacral spinal procedure), a shoulder procedure, an elbow procedure, a wrist procedure, a hip procedure, a knee procedure, an ankle procedure, a foot procedure, a hand procedure, or other procedures. In some embodiments, the procedure can be a total knee arthroplasty (TKA procedure) or a hip replacement procedure.

In some embodiments the gels can be delivered in to relieve pain, including in a joint, as an alternative to a surgical procedure or a way to delay the need for a surgical procedure.

In some embodiments, a gel can be delivered to or proximate a femoral nerve. In some embodiments, gel can be delivered to or proximate a sciatic nerve, including a popliteal sciatic nerve.

In some embodiments, gel can be delivered to the adductor canal alone, or additionally proximate the sciatic nerve, such as to control early postoperative pain after total knee arthroplasty. The articular branches, arising from the main trunks of the tibial and obturator nerves, travel through a tissue space between the popliteal artery and the femur to innervate the posterior capsule of the knee. These articular branches can be neuromodulated by infiltrating this tissue plane between the popliteal artery and the capsule of the knee (iPACK) with a gel under ultrasound or other imaging guidance. As such, an iPACK procedure can selectively modulate only the innervation of the posterior knee joint while sparing the main trunks of tibial and common peroneal nerves, thereby, maintaining the sensorimotor function of the leg/foot.

In some embodiments, a gel can be delivered to the epidural space. In some embodiments, a gel can delivered proximate the spinal nerves in the cervical, lumbar, thoracic, or sacral regions of the spine to treat, for example, back pain.

Hip. Without being limited, these neural targets are high articular nerve ablation whose targets are: the femoral and obturator nerve branches of the anterior hip joint, the nerve to the quadratus fermoris and the superior gluteal nerve to the posterior hip joint. The anterior nerves of the hip are targeted for severe non-operative hip pain. For example, the ablation over the superior portion of the acetabulum and inferomedial portion of the acetabulum. The neural target is selected based on the source of the pain—the obturator nerve for the anteromedial joint, the femoral nerve for the anterolateral joint, the sciatic nerve for the posterosuperior joint, the nerve to the quadratus femoris muscle for the posteroinferior joint, and the superior gluteal nerve for the posterolateral joint.

Shoulder. Chronic shoulder pain may be attributed to rotator cuff disease, glenohumeral joint osteoarthritis, nerve injuries and capsulitis. The superior trunk, suprascapular nerve, the suprascapular nerve medial and lateral trunks, axillary nerve (AN), lateral pectoral nerve (LPN), subscapular, supraclavicular nerves can be targeted preceding or as an alternative to shoulder surgery in patients with chronic shoulder pain. For example, the posterior glenohumeral joint (GHJ) innervation is from the axillary and suprascapular nerve. Distal banches of the posterior division of the axillary nerve can be targeted. The anterior-superior GHJ can be targeted via the nerve to the subscapularis. Lastly, the GHJ can be targetd by the lateral pectoral nerves. Newer blocks include delivering to the suprascapular nerve via the spingolenoid Notch under fluoroscopy or ultrasound, posterior access to the axillary articular nerves, anterior access to the lateral pectoral nerve branches over the coracoid process. Articular branch nerves are also potential targets. Mixed motor nerves can be targeted in patients that have limited motor use of the shoulder. However, if possible, locations with complete motor sparing potential are desired. For these locations, the hydrogel can be highly localized to deliver only to the sensory branches to reduce off target injury that would potentially occur with a radiofrequency or chemical ablation approach where the spread is more difficult to control. Contract dye can be bound to or incorporated into the hydrogel to confirm target delivery and prevention of off-target spread. These nerves can also be targeted for post-stroke shoulder pain, such as the axillary nerves. Other indications include chronic hemiplegic shoulder pain, rotator cuff disease, osteoarthritis of the GHJ, nerve injuries and capsulitis.

Knee. Nerve that innervate the knee include the superolateral genicular branch from the vastus lateralis, superomedial genicular branch from the vasus medialis, the interfomedial genicular branch from the saphenous nerve, and the terminal branch of the nerve vastus intermedius. Other targets include inferomedial, infrapatellar, medial retinacular, superolateral and superomedial genicular nerves (https://www.ncbi.nlm.nih.gov/pubmed/30271194).

Other targets include head and neck pain-mandibular, maxillary, lesser palatine. Other shoulder and proximal humerous include interscalene nerve. For elbow pain, the supraclavicular, infraclavicular, axillary, median and ulnar nerves are targeted. For thorax and breast pain, thoracic paravertebral, intercostal and intrapleural nerves are targeted. For abdomen and inguinal pain, in addition to the paravertebral gutter, the transversus abdominus plane and the quadratus lumborum are targeted with the neuromodulatory hydrogel. Many of the The in situ forming hydrogel can be delivered in conjunction with the delivery of electromagnetic energy, such as RF (standard, cooled, pulsed RF), cryoablation, hydrodissection, microwave, and/or ultrasound energy to a desired anatomic location, such as a peripheral nerve, for example. In some embodiments, neuromodulation can include delivering a gel, such as a hydrogel to the paravertebral gutter. The hydrogel could include, for example, an in situ polymerizing or crosslinking hydrogel, or an injectable hydrogel slurry.

The neuromodulation could be unilateral or bilateral, e.g., on the left side, right side, or both, and can be stepwise or within the same operative procedure.

Also disclosed herein is a method of modulating the nerves of a patient, wherein neuromodulating comprises flowing a gel comprising a therapeutic agent within the epineurium of a nerve or adjacent to the nerve. The neuromodulatory gel may be delivered circumferentially around a nerve or may sit as a bolus of gel directly adjacent to the nerve. The therapeutic agent could include a neurolytic agent, such as, for example, a non-depolarizing agent. The neurolytic agent could prevents or block the release of norepinephrine, substance P, or other neurotransmitter, and/or be co-administered with a blocking agent. The therapeutics agent could be a nerve blocking agent, blocking the transmission of nerve signals back to the central nervous system. Some examples of neurolytic agents that can be used include, for example, nifedipine, lamotrigine, minoxidil, reserpine, tetrabenazine, amiodarone, dextromethorphan, valproic acid, mecamylamine, phenoxybenzmine, alfuzosin, haloperidol, desipramine, bretylium tosylate, doxepin, bupropion, taxol, and oxaliplatin. The agent could be combined with an anesthetic in the hydrogel or the agent could be loaded together with a solubilizer such as ethanol in the hydrogel. A gel could have any desired porosity, such as less than about 50 μm, 20 μm, 10 μm, 5 μm, or even less. The gel could include a biodegradable or bioerodable polymer, or an injectable hydrogel. The gel could include any number of the following characteristics: in situ forming, in situ crosslinking, biodegradable hydrogel. The gel can be delivered blindly, under ultrasound or fluoroscopic guidance. In the peripheral nervous regions into which it is injected, a longer hydrogel gelation time of 10 to 20 seconds permits longitudinal spread within the fascial plane in which the nerve runs to cover a region of nerve between 1 and 20 cm along the length of the nerve, more preferably, 1 and 10 cm along the length. In some embodiments, the gel volume is between 0.5 and 20 cc, preferably 0.5 and 5 cc, more preferably 0.5 and 2 cc. The nerves travel course through these fascial planes between muscles, tendons and bones with is a fascial plane before entering the target tissue. The nerves may course together with blood vessels (arteries and veins) or travel in their own plane. Adipose tissue and other connective tissue may be present. The tissue space around the nerve may be hydrodissected first using 1 to 10 ml saline or anesthetic solution. This assists with separating the tissues around the nerves to permit gel spread along the nerve bundle. The volume of hydrogel delivered may be adjusted based on the delivery target, the diameter of the nerve, and the duration of drug release required, if any.

At least about 50% of the volume in the fascial plane is desirable, preferably more than 90%, allowing the in situ forming hydrogel to spread through the tissues prior to forming a gel around the nerve. In some embodiments, the gel has a volume of between about 2cc and about 30cc, such as between about 0.1 cc and about 10cc. The gel can be delivered unilaterally or bilaterally, such as in a rostral or caudal direction, or both.

In many applications, ultrasound or fluoroscopic guidance allows for more precise delivery of the gel to the target nerve. Many nerves, such as the sciatic nerve, are directly visualized under ultrasound and routinely accessed to deliver anesthetics as part of procedures requiring local infusion of anesthetic. Other nerves, such as the genicular nerves, are not directly visible under ultrasound or fluoroscopic guidance, and so hydrogel delivery to these regions is based on anatomical landmarks such as bone (fluoroscopic) and vascular structures that frequently are adjacent to the nerves (ultrasound, doppler, CT guida). For direct delivery to peripheral nerves, the needle can be delivered directly adjacent to the nerve and the hydrogel deposited here. In doing so, a smaller volume of hydrogel (e.g. 0.5-3 ml, more preferably 1 ml) can be delivered directly adjacent to and/or circumferentially around the nerve. In applications in which a nerve or nerves can not be localized, a larger volume of gel (e.g. 5-10 ml) can be delivered to ensure that the entire potential space is filled and the nerve is captured. In regions in which nerves are traveling through narrow tunnels (e.g. carpal tunnel), smaller volumes of gel may need to be delivered to achieve the desired therapeutic result.

In some embodiments, neuromodulation through the hydrogel is achieved through the delivery of an agent in the hydrogel, either embedded in the hydrogel or embedded in particles (micelles, nanoparticles, microparticles) from which it is released in a more sustained fashion. In some embodiments that neuromodulation agent is a neurostimulatory, neuroinhibitory, neuroprotective, neuroregenerative, neuron survival, neuronal differentiation, or neuroablative agent.

In some embodiments disclosed herein is a method of selectively modulating a specific group of nerves within a patient by delivering an agent within the hydrogel, such as the type A, type B, type C sensory fibers, all of the afferent fibers, autonomic, or the motor fibers (e.g. gamma motor neuron). The majority of the peripheral nerves bundles contain a mix of the sensory, motor, and autonomic fibers. Of particular interest are targeting sensory nerve fibers and specifically pain fibers in mixed nerves (nerves that contain motor and sensory fibers). The selectivity is conferred by the type of drug that is released from the hydrogel. The selectivity is not necessarily required when delivering the hydrogel to pure sensory nerves.

In some embodiments, the drug-loaded hydrogel is directed towards the target nerve and a second hydrogel is delivered as a spacer to protect off target structures, particularly other groups of nerves, from the effects of the target agent. Alternatively, any target tissues and nerves that require protection can first be delivered with a bolus of the blank hydrogel prior to the delivery of the drug loaded hydrogel. For example, in one embodiment, a blank hydrogel is delivered first around the recurrent laryngeal nerve and then the drug-loaded hydrogel is delivered to the target stellate ganglion. In addition, the delivery needle/catheter can include, for example, at least one energy delivery effector, such as an RF electrode, microwave antenna, ultrasonic transducer, and the like.

Also disclosed herein in some embodiments is a hydrogel for use in other anatomical locations as disclosed herein. The hydrogel can include, for example, a neurolytic active agent; and a biodegradable polymer. The gel could include a biodegradable or bioerodable polymeric injectable hydrogel.

### BRIEF DESCRIPTION OF THE DRAWINGS

FIG. 1 illustrates an embodiment of a therapeutic agent delivery system including a catheter that can interface, e.g., removably interface with a syringe or other therapeutic agent housing.

FIG. 2 illustrates a distal portion of the catheter.

FIG. 2A is a cross-section through line A-A of FIG. 2.

FIG. 2B is a cross-section through line B-B of FIG. 2.

FIG. 2C is a cross-section through line C-C of FIG. 2.

FIGS. 3-3C are views of a schematic illustration of a delivery catheter system including one or more therapeutic agent housings (e.g., syringes) removably connected to a catheter configured to deliver a plurality of therapeutic agents into different anatomical locations.

FIGS. 4-4A are schematic illustrations of a delivery catheter system similar to that illustrated in FIG. 3, except the first therapeutic agent housing has only a single chamber fluidly connectable via a first input port on the catheter to a first lumen.

FIGS. 5A-H schematically various embodiments of methods of injecting a gel into various locations within or proximate a nerve

### DETAILED DESCRIPTION

The invention relates in some aspects to the delivery of in situ forming hydrogels to nerve to treat a variety of conditions related to peripheral nerves. In some embodiments, it is desirable to deliver a hydrogel to nerves to 1) encourage nerve regeneration, 2) to block nerve regeneration, 3) to encourage neuronal survival and reduce inflammation, 4) to provide a temporary protection for a nerve until a more definitive procedure can be performed, 5) to deliver agents to the nerves to block pain regeneration, 6) deliver agents to release neurogenic inflammation 7) delivery agents that block neurotransmitter release (e.g. neuropeptide Y, calcitonin gene-related peptide (CGRP), secretoneurin, the tachykinins (TK), ATP, substance P (SP) and neurokinin A (NKA), norepinephrine, acetylcholine, nitric oxide, vasoactive peptide (VIP), and encephalin have also been localized to pre-ganglionic nerve endings, 8) delivery of agents that block receptors on the surface of the nerves (TRPV1, TKA, NK1, NK2)

Some peripheral locations, e.g. the tibial nerve, have been linked to autonomic reflex loops whereby modulating the tibial nerve will have beneficial effects for the patient with abdominal pain or gastrointestinal/genitofemory conditions.

Procedure. One of the advantages of percutaneous image-guided procedures is that they do not require any special equipment. Needles approximately 80, 100, 110, 150 mm long may be suitable depending on the depth of the target (e.g. Pajunk needles). If there is concern for inadvertent vascular puncture, the syringe can be withdrawn to confirm no blood is present in the syringe barrel (negative aspiration test). Contrast agent may be delivered with the hydrogel to assist with visualization of the spread of the hydrogel.

Neurostimulation can be incorporated into the procedure, particularly procedures in which a neurolytic agent is delivered in the hydrogel. For the applications, neural targets are typically sensory nerve bundles and so a) confirmation of the absence of motor stimulation and b) stimulation of the sensory nerves provides additional confirmation of the neural target. The neurostimulation technique involves using an insulated 18-gauge or 22-gauge needle connected to a nerve stimulator delivering a current of 2.5 to 5.0 mA with a pulse duration of 0.1 milliseconds and a frequency of 2 Hz. When the needle is near the nerve bundle, a response is elicited. In the case of motor stimulation, the intensity of contraction is generally recognized to be related to the distance between the needle and the intercostal nerve. The ideal position of the needle is when the muscle response is maintained with a current less than 0.5 mA.

A low frequency ultrasound probe can be connected to an ultrasound machine (e.g. S-Nerve, Sonosite).

In some embodiments, combined anesthetic and neurolytic-hydrogel injections are made. The anesthetic can serve both as a pain reliever as well as to confirm the correct placement of the needle within the paravertebral gutter. If, upon injection, the anesthetic causes deflection of the pleural as viewed under fluoro or ultrasound guidance, the needle is considered to be in the right location. In some embodiments, the neurolytic-hydrogel delivery may be performed over several days in order to reduce the likelihood of neuritis. In some embodiments, the target can be the stellate ganglion.

Peripheral indications. Non-limiting examples of indications that would benefit from short term or long term delivery of an agent in an in situ forming hydrogel include vascular insufficiency/occlusive vascular disorders, lymphedema, neurogenic inflammation, diabetic neuropathy, painful diabetic neuropathy, idiopathic peripheral neuropathy, testicular pain, knee osteoarthritis, idiopathic facial pain, pain from shingles, phantom limb pain, residual limb pain, amputation-related pain, nerve entrapment, surgical trauma, nerve ischemia, critical limb ischemia, amputation stump pain, pain following mastectomy, Bell's palsy, orofacial pain syndrome including neuropathic orofacial pain, vascular headache and sympathetically maintained headaches, neuropathic pain syndromes in cancer pain, sudden infant death syndrome, endometrial and/or peritoneal pain, anorexia nervosa, thoracic outlet syndrome, arthritis, posttraumatic sympathetic dystrophy, thromboangitis obliterans, diabetes and insulin resistance, particularly at early stages, collagen disease such as scleroderma, ischemic stroke and subarachnoid hemorrhage. Sympathetic denervation may also be beneficial in, for example, the treatment of inflammatory diseases or diseases with an inflammatory component such as acute inflammation, shock (hypovolemic, septic, neurogenic), sepsis, and acute respiratory distress syndrome by reducing neutrophil and natural killer cell counts and improving lymphatic flow, glaucoma, facial blushing of hyperhidrosis (T1 to T3, or T2 only), palmar hyperhidrosis (generally T2-T3), axillary hyperhidrosis (generally T2-T4), reflex sympathetic dystrophy or complex regional pain syndrome, dry eye or mouth disorders, pulmonary embolism, ischemic or diabetic ulcers, limb ischemia or leg pain, vasospastic disorders, causalgia, peripheral arterial disease or occlusive arterial disease, burning pain accompanied by hyperpathia, hyperaesthesia, and hypercoagulative/prothrombotic states. In some embodiments, endpoints include, at least an amelioration of the symptoms associated with the pathological condition afflicting the host, where amelioration is used in a broad sense to refer to at least a reduction in the magnitude of a parameter, e.g. symptom, associated with the pathological condition being treated. In some embodiments, the endpoint can be reduction in a symptom such as anxiety and/or panic. In one embodiment, the treatment of hot flashes results in a reduction in mean hot flash scores and frequencies, such as measured by the Hot Flash Related Daily Interference Scale for example. Within these diseases, targeting of the stellate ganglion alone may be sufficient for treatment of CRPS (type I and II) of the upper extremities, chronic and acute vascular sufficiency, occlusive vascular disorders of the upper extremities, poor lymphatic drainage, edema of the upper extremity following breast surgery, post-herpetic neuralgia, sudden hearing loss, hyperhidrosis, Bell's palsy and orofacial pain, trigeminal neuralgia, vascular headache such as cluster and migraine headaches, phantom limb pain or amputation stump pain.

Chronic pain management. Long-duration or permanent therapy directed towards the peripheral nerves can be used for the treatment of chronic pain, whether from the sustained delivery of drugs from days, weeks or months or for patients seeking more permanent relief through the ablation and prevention of regeneration of the sensory nerves involved in propagating the pain.

### Visceral Targets.

Lower thoracic paravertebral blocks can also be performed for the treatment of hypertension, heart failure (systolic, diastolic), and/or hypertensive crises. The paravertebral blocks can also be extended through the administration of larger volumes to achieve a block of the splanchnic nerves as the neurolytic agent-gel spreads outside of the paravertebral gutter proper. These blocks may include a mixture of the neurolytic agent and anesthetic in the gel or an anesthetic delivered prior to the delivery of the neurolytic agent-gel. By performing one or more injections to achieve gel delivery from T11 to L2, it is possible to treat various diseases.

Splanchnic nerve blocks can also be effective for blocking the visceral nervous pathways to the kidneys, adrenal and heart for the treatment of hypertensive crises, high blood pressure including essential hypertension, diastolic heart failure and others. A splanchnic block with an anesthetic (e.g. novocaine or bupivacaine) can be performed as a prognostic test prior to the operation. Not to be limited by theory, one advantage of paravertebral block over splanchnic block is that there is less likelihood of a marked lowering of blood pressure.

In some embodiments, gels can affect nociceptive neurons. In some embodiments, gels can disrupt visceral afferent fibers that travel through the sympathetic chain to the dorsal root ganglion. In some embodiments, gels can disrupt visceral and visceral afferent and somatic afferent communication directly, while advantageously avoiding intercostal neuritis. In some embodiments, paravertebral block can be performed for the treatment of herpes zoster lesions, performed at the level of the skin eruptions, to provide immediate and permanent relief. In some embodiments, thrombophlebitis, phantom pain, and reflex sympathetic dystrophy can also be treated.

Delivery of the drug-loaded hydrogel to the sympathetic chain for the treatment of visceral pains as disclosed in U.S. Pat. No. 9,855,317 to Bright, which is incorporated by reference in its entirety. Specific levels at which the sympathetic chain should be targeted to achieve pain reduction are included as follows: Pain of the parietal peritoneum is supplied by the intercostal nerves and the lumbar nerves which the visceral organs appear to be insensitive. The abdominal viscera contain sensory nerves of sympathetic origin that are sensitive to squeezing, pricking, cutting and other stimuli. These include the greater and lesser omentum, the bile ducts and portal blood vessels. The gastro-intestinal wall, liver, spleen and gall bladder appear to be less sensitive, but can be targeted in some cases. The parietal peritoneum, mesenteric root, porta hepatica are generally all sensitive, and can be targeted with gel and other therapies. The gallbladder, kidney, stomach, and appendix can also be treated using systems and methods as disclosed herein, non-limiting target locations of which will be described. For the stomach, the T6 to T8 or T9 levels bilaterally may be targeted. In the presence of a gastric ulcer, pain may be relieved by an T7-T8 injection on the right if the ulcer is closer to the pylorus or the left if it is closer to the cardia. For other conditions with low motility or lower acidity, paravertebral block can potentially result in increased acidity and motility. The pylorus can be treated by modulation of T7 on the right and sometimes T7-T8; and the duodenum by T7 on the right. The kidney can be modulated for example via T11 or T12 to L1 or L2 on the ipsilateral side (kidney pain can respond well). Injections into the paravertebral space and splanchnic nerves may not only help reduce colic but also improve urine output in patients with reflex anuria and oliguria. Gel injections, for example, can also restore normal renal secretion. The appendix can be treated by modulation of T12 to L3 on one or both sides. For the pancreas, T8 to T10 on the left side and may be effective for the treatment of symptoms of pancreatitis.

Gallbladder interruption is affected by injection from T9 and T10 on the right side. The gallbladder is innervated from T8 to T9 spine segments and passes via the splanchnic and celiac plexus to the hepatic plexus to form a network around the hepatic artery and the bile ducts, accompanying the blood vessels to the gallbladder wall. Although pain may radiate to the right abdominal wall, right lumbar, or right shoulder, injection into the 10th thoracic nerve at T9 or T9-T10 can in some cases immediately relieves the pain.

The liver and gallbladder can be treated via modulation of T6 to T9. The small intestine can be treated via modulation of T9 to T11; the ascending colon via modulation of T11 to L1; and the ureter via modulation of L1 to L2.

The pylorus and duodenum receive their nerve supply from the right side. Although the vagus and the splanchnic nerves supply the celiac artery bilaterally. However, the pancreas typically receives unilateral innervation from the left side.

Abdominal diseases: Successful therapy to the paravertebral gutter can relieve permanent pain, tenderness to pressure, colic, and may also interrupt visceral reflex tension (parietal reflex tension) of the abdominal wall making palpation possible. This includes peritonitis of the abdominal wall.

In this manner, gastric pain can be treated by paravertebral injections from T6 or T7 to T8 or T9, gallstones from T6 to T10 or T11, and renal colic from T10 to T12 or L2. Gastric pain may be from inoperable carcinoma of the stomach, gastritis, ulcers. In particular, kidney pain can respond well to modulation as described herein.

Additional Indications. The sympathetic nervous system has also been demonstrated to play a role in hemostasis via procoagulant factor activation (fibrinogen, FVIII, vWF, and platelet activity) as well as fibrinolytic activity (t-PA reduction) and thus may contribute directly to the development of coronary artery disease and myocardial infarction. The infusion of adrenergic neurotransmitters, for example, increases blood coagulation, fibrinolysis, and platelet activation and even more so in patients with atherosclerotic disease and chronic stress. Thus the downward modulation or neurodestruction of the sympathetic nervous system, as can be achieved through denervation of the upper and/or lower thoracic sympathetic chain.

Coronary insufficiency leads to anoxia and ischemia of the heart muscle and results in cardiac pain which may be caused by both primary cardiac and systemic disorders including atheroslerosis, syphilis of the aorta, embolism, free aortic regurgitation—this is believed to be due to lowered diastolic pressure, anemia, paroxysmal tachycardia in which the rapid heart rate calls for greater coronary flow than the heart can delivery, hyperthyroidism resulting in increased metabolic demands and an excess of cardiac work beyond the coronary reserve. Other conditions that mimic pain in the chest include an aneurysm of the thoracic aorta, aortic valvular disease, cholelithiasis, pericarditis, and gallbladder disease may also be treated. Generally, visceral pain can be treated by ablating the visceral afferents or interoceptors that innervate that organ or tissue.

Neuromodulation of the splanchnic nerves may also be desirable to treat renal ischemia, hypertension, heart failure, angina, enlargement of the heart, abnormalities in electrocardiograms, reduce the likelihood of cerebral accidents, as well as retinopathy, papilledema and hemorrhages or exudates. In the 1930s, thousands of patients underwent bilateral supradiaphragmatic splanchnicectomy. In some cases, the greater splanchnic nerve and some or all of the 5th to the 12th sympathetic ganglia were also resected bilaterally. The lesser splanchnic nerves can also be avulsed or divided.

Any of the previously mentioned surgical operations for sympathectomy may be modified to deliver a neuromodulatory agent in a gel formulation including anterior root ramisection, subdiaphragmatic approach to the sympathetic nerves, supradiaphragmatic approach, or celiac ganglionectomy with the transabdominal approach.

If subdiaphragmatic, injections can be made into the first and second lumbar ganglia.

In splanchnic embodiments, hydrodissection with saline can be advantageous to achieving more extensive flow of the neuromodulatory gel to the splanchnic nerves.

In addition, the first and second lumbar sympathetic ganglia as well as the greater and lesser splanchic nerves, as necessary, can be targeted to denervate the adrenal medulla. These are pregagnlionic fibers.

Lumbar symapthectomy can be performed for end stage arterial occlusive disease, vasospastic disorders, causalgia or complext regional pain syndrome, or claudication.

The lumbar sympathetic chain varies widely in its morphology and so although there are typically 3 ore more sympathetic ganglia (between one to 4 identified).

Other targets. Local delivery for pain management. The therapy may also be delivered locally to nerves for the amelioration of symptoms related to peripheral neuropathies. A neurolytic agent delivered in a gel can be preferred in some embodiments since allows for reduction in the volume of agent delivered as 1) the drug can remain in contact with the nerve (ganglia, plexus, nerve fibers) at a neurolytic concentration for longer than an agent that washes over the nerve and spreads elsewhere. In one embodiment, the stellate ganglion block is performed with 1-3 ml of neurolytic/anesthetic agent in a gel as opposed to the 5-20 ml of anesthetic delivered today which is well documented to travel to many other off-target neural structures. Similarly, in one embodiment 5 to 10 ml of the therapy is delivered to the celiac plexus instead of the 10-30 ml of alcohol that is injected in the region today. This can significantly reduce the spread of a toxic agent through the retroperitoneum and potentially result in longer relief of symptoms. Other targets include any of the locations that anesthetic blocks are placed currently such as the brachial plexus, retrobulbar, hypogastric plexus, nerves innervating the pectoral and serratus anterior, transversus abdominis, saphenous nerve, femoral nerve, blocks at elbow and arm, axillary block, infraclavicular block, suprascapular and axillary nerve block, supraclavicular block, interscalene block, and endoscopic or transabdominal blocks, or blocks for neuropathy. In some embodiments, if the relief of pain is immediate upon injection of an anesthetic (e.g., novocaine), then injection of the neurolytic agent and/or gel can be made. In another embodiment, the neurolytic agent may be injected into the space first and then a second procedure performed to inject a blank gel to block regeneration.

In some embodiments, gels can be delivered to or proximate motor nerves, sensory nerves, differential nerves, autonomic nerves, brachial plexus nerves (e.g., axillary, interscalene, supraclavicular, infraclavicular), individual upper extremity nerves (e.g., median, radial, ulnar, musculocutaneous, axillary), sciatic nerve, ankle nerves, metatarsal nerves, oral nerves, femoral nerves, popliteal fossa nerves, saphenous nerve, distal nerves, digital nerves, deep peroneal nerves, superficial peroneal nerves, tibial nerves, sural nerves, and the like.

In some embodiment, gels including those disclosed herein can be delivered to one, two, or more somatic nerves but not autonomic nerves (e.g., sympathetic or parasympathetic nerves). In some embodiments, a gel can be delivered to one, two, or more somatic nerves as well as autonomic nerves. In some embodiments, gels including those disclosed herein can be delivered to or around nerves and utilized for treatment and/or prevention of acute or chronic pain, either in conjunction with an associated operative procedure or procedures, or without an associated operative procedure. In embodiments where the delivery of the gel is associated with an operative procedure, the gel can be delivered either preoperatively (e.g., within about 4 weeks, 3 weeks, 2 weeks, 1 week, 6, 5, 4, 3, 2, or 1 day), perioperatively (e.g., prior to but on the same day as the procedure, intraoperatively, or postoperatively on the day of the procedure), or postoperatively (e.g., about or at least about 1, 2, 3, 4, 5, 6, 7 days or more after the procedure). In some embodiments, the gel can include any number of therapeutic agents including anesthestics, neurolytic agents, and other neuromodulatory agents including neuroablative agents, such as those disclosed elsewhere herein.

In another embodiment, a patient that responds to an equivalent anesthetic block is considered a potential responder for the therapy and qualifies for receiving the therapy. In one embodiment the block is performed, providing local anesthetic to the region and providing verification that the patient is likely going to be a responder to the therapy, and then the patient receives the neurolytic agent-hydrogel. In another embodiment, the patient receives a block and then comes back 2-6 weeks later when the block has worn off and/or the symptoms have returned.

Direct/indirect. Neuromodulation can be achieved via direct or indirect application of energy or neuromodulatory agents to target neural matter, to adipose or fascial tissue that contains the neural matter or to vascular structures that support or intersect the target neural matter.

Targeting of specific arm. The therapy may be directly towards a purely efferent or afferent nerves whether through spatial constraint of the delivery of the therapy to a specific region, such as the anterior/ventral nerve root or posterior/dorsal nerve root, respectively, or through the delivery of a neuromodulatory agent that has preferential affinity or stronger effect on one type of nerves over another, such as for sympathetic efferents over sympathetic visceral afferents. Alternatively, a mix of afferent and efferent fibers may be targeted as they run their course wrapping longitudinally along a blood vessel to the thoracic organs or simply crossing a blood vessel on their way to the thoracic organ. In some embodiments a specific neuron, soma, axon, or nerve fiber, nerve plexus/plexi or nerve bundle may be targeted. In addition, the methods and apparatus described herein may, for example, be used to modulate parasympathetic nerves and/or the central nervous system including the brain and spinal cord.

Numerous studies have demonstrated the aberrant connections between sympathetic fibers and the dorsal root/dorsal root ganglia in disease. Sympathetic nerves have been demonstrated to sprout into the dorsal root ganglia and form abnormal basket-shaped terminal arborizations around some neurons. This sympathetic-sensory coupling is thought to play a major role in neuropathic pain that occurs after injury to peripheral or central nerves.

By delivering a neuromodulatory gel or blank hydrogel (containing no active ingredient) percutaneously to this region within a specified time period, e.g., a week or two after peripheral nerve injury, effectively in a prophylactic procedure, the diffusion of factors such as nerve growth factor and/or leukemia inhibitor factor or interleukin-6 may be interfered with, thereby preventing the formation of these aberrant connections and thus reducing or preventing the development of neuropathic pain. The hydrogel can provide a barrier to the diffusion of these high molecular weight proteins and/or challenges the ability of the sympathetic neurons to sprout towards the gradient of trophic factors released by the DRG.

Thus, the hydrogel can be delivered into the paravertebral gutter using a more lateral percutaneous approach so that the material spreads from the needle tip into the intervertebral foramina and thus to the dorsal and ventral roots. Alternatively, a catheter can be utilized to deliver the gel formulation such as a coiled catheter developed by Cedric Luyet the SonoLong Curl Sono Set (Pajunk Holding GmbH, Geisingen, Germany) or other catheter can be extended from the needle such that the catheter tip is adjacent to the intervertebral foramen. In another embodiment, the hydrogel formulations may be injected into the epidural region that contains the dorsal and ventral roots. This region may be referred to as the epidural root sleeve and injections of anesthetics here are referred to as root sleeve blocks, root blocks, or transforaminal epidural blocks. The hydrogel may be delivered to this region in the same manner that these root blocks are performed. For this application, a longer indwelling biodegradable hydrogel with minimal swelling may be preferable given the proximity to the spinal cord and the desire to prevent the formation of long-term aberrant connections. The dura acts as a barrier to prevent diffusion of the hydrogel to the paravertebral space. The targeted epidural levels are based on the clinical indication. For example, for cardiac applications, T1 to T4 may be targeted.

Similarly, after myocardial injury, cervical and upper thoracic dorsal root ganglia may sprout to innervate adjacent sympathetic nerves, such as those located in the cervical sympathetic ganglia and upper thoracic sympathetic ganglia. In one embodiment, brilliant blue G, a selective P2X7 antagonist or other P2X receptor antagonist (e.g. P2X7, P2X3, P2X2 etc), is administered to the stellate ganglia and adjacent C8 and/or T1 dorsal root ganglia in the hydrogel in order to inhibit the activation of glial cells and thus the sensory-sympathetic coupling. By targeting the stellate and upper thoracic ganglia (T1 to T4 or T5), diseases such as coronary artery disease, hypertension, angina, and heart failure can be treated. For example, in patients that are at high risk for a myocardial infarction or another myocardial infarction or arrhythmias, the paravertebral region may be filled with a sustained release drug delivery system up to and/or including the intervertebral foramen to treat cardiac disease. In some embodiments, the delivery of these neuromodulatory agents locally may be desirable in the superior, middle, intermediate, or inferior/stellate ganglia to modulate cardiac function. As the P2X3 receptors are thought to play a critical role in nociceptive signaling in chronic pain syndromes, it follows that the delivery of these drugs to other levels or regions where thoracic, lumbar, or sacral sympathetic nerves may form aberrant connections with dorsal root ganglia or afferent nerves may be desirable.

If these aberrant connections have already formed, then the denervation of fibers between the dorsal and ventral root or the dorsal root and the sympathetic ganglia can be achieved with the neurolytic-loaded gels described. In addition, pathologic connections between the sympathetic fibers and the dorsal root may be prevented with the delivery of an impenetrable hydrogel between the dorsal and ventral root. In some embodiments, resiniferatoxin (RTX), discussed in more detail elsewhere herein, may be delivered in the gel in order to target the therapy to the dorsal root ganglion.

In some embodiments, the active agent may be delivered prior to the delivery of the gel. In another embodiment, ethanol may be delivered prior to the delivery of the drug-loaded gel in order to dissolve the adipose tissue in the region prior to the delivery of the gel.

Paravertebral to transformainal/root. Injections into the paravertebral space may also be directed towards (based on the needle tip) the transforaminal space with the specific intent to deliver the gel to the dorsal and/or ventral roots (effectively a paravertebral+sleeve therapy).

Paravertebral to Splanchnic. While some embodiments herein are directed towards retaining the hydrogel within the paravertebral space proper and avoiding dissemination of the hydrogel to the intercostal spaces, in some embodiments it may be desirable to increase the spread of the hydrogel beyond the paravertebral space. This can be achieved by increasing the volume of injectate to overwhelm the volume of the paravertebral space and cause overflow into the adjacent segments. In one embodiment, the autonomic efferent and afferent nerves of the lower thoracic regions are infiltrated with the formulation by positioning the needle in the paravertebral space and then delivering between about 5cc to 30 cc, more preferably 15-20 cc into the paravertebral space. For example, dye experiments have demonstrated multi-level coverage of the splanchnic nerves coming off of the sympathetic chain. Thus, paravertebral and prevertebral neuromodulation can be achieved, directly or indirectly.

Intercostal. In yet another embodiment, a gel may be delivered to the paravertebral gutter through a more lateral injection into the intercostal space, a method referred to an intercostal injection or by some as a lateral thoracic paravertebral block. For example, the injection may be performed, for example, about 8 cm from the midline. One approach is to deliver the agent between the internal intercostal membrane (continuation of costotransverse ligament), the pleura, and the transverse process. Another approach is the transversus abdominis plane (TAP) proximate the thoracic wall muscles. By delivering the active agent in a gel, the agent will not spread as far through the subpleural space (longitudinally) and instead travel medially to the paravertebral space. Intercostal blocks may also be referred to as thoracic wall blocks which can be used for surgical anesthesia and postoperative analgesia for breast surgery.

Stellate approach. An anterior paratracheal approach is typically performed at the level of the anterior tubercle of C6 (Chassaignac's tubercle) which is in proximity to the middle cervical ganglion. The cervical sympathetic trunk lies deep in the prevertebral fascia on the surface of the longus colli muscle. Using ultrasound to guide the inection, the needle can be targeted to the subfacial plan and the appropriate spread of anesthetic can be confirmed. The injectate will spread more caudally if placed in the correct subfacial plane and requires smaller volumes of injectate. By utilizing a gel, the spread of agent is limited and the block becomes more selective and specific.

Sequencing. In some embodiments, the hydrodissection of the paravertebral space is desirable to facilitate the improved rostrocaudal spread of the formulation. Fluid dissection or hydrodissection can be achieved, for example, with saline, hyperosmolar solutions such as hypertonic saline, ethanol, or local anesthetic (e.g. bupivicaine, ropivicaine, and the like). Contrast may be added to confirm the appropriate spread of the solution. In some instances, it may be desirable to inject a large volume of saline to facilitate the creation of the potential space in the paravertebral gutter. This can be performed, through the administration of 2 to 15 ml of agent into the paravertebral gutter. A single level hydrodissection can also be achieved with smaller volumes, such as with between about 1 to 5 ml. Similarly, by delivering ethanol or phenol, for example, prior to the administration of the gel, the neurons and surrounding fat can be pre-ablated prior to the delivery of the gel.

Terms that describe modulation of the nervous system or neuromodulation refer to providing excitatory, inhibitory, blocking signals to the nerve increasing or decreasing the likelihood for an action potential, the modulation of the nerve to improve its survival or result in its cell death, the stimulation of a nerve to regenerate, the stimulation of a nerve to fire action potentials such as nerve stimulation, and/or result in the up- or down-regulation of proteins including neurotransmitters, receptors through viral or non-viral vectors delivering DNA, RNA, siRNA, microRNA (miRs), modified messenger RNA, or antibodies.

Gel delivery including any therapeutic agent, such as those disclosed elsewhere herein, can be utilized concurrent with, prior to, or following a neuroablation procedure, including those described elsewhere herein. More widespread adoption of local neurolysis or neuroablation to treat painful peripheral and visceral conditions has been limited by undesirable side effects from incomplete neurolysis, unpredictable and variable efficacy, and lack of durability of effect. Incomplete or partial neuroablation occurs when the energy modality or neurolytic agent are only partially effective at killing the nerves and a portion of the nerves are damaged but the axons are intact such that they can recover. By delivering an agent to the site for a longer period of time, such as can be achieved with a neurolytic agent in a formulation, it is possible to more completely ablate the target nerves.

Secondarily, variable efficacy and unpredictable and non-sustained durability of efficacy are related in part to nerve regeneration. In the periphery, this occurs when transected, crushed, or ablated nerves regenerate into the distal nerve stump (providing an autologous scaffold, pro-regenerative factors, and guidance) and reinnervate the target tissue. This can also occur when damaged nerves innervate inappropriate or non-target tissues or, as is increasingly appreciated, sprout and form inappropriate connections with other nerves.

The use of a biocompatible, injectable therapy that can be delivered in combination with or subsequent a neural denervation therapy can be desirable, regardless of the method of denervation (ultrasound, radiofreqency, microwave, cryotherapy etc.). Alternatively, the injectable therapy can be delivered first, prior to the neurolytic agent or denervation approach in order to limit the spread of the neurolytic agent or e.g., thermal, cryo, or other agent to the target site. In limiting the peripheral damage outside of the target tissue and extending the duration of effectiveness, the pain and safety risks associated with the procedures are more effectively offset. Also, by combining the delivery of a neurolytic agent either prior to or concomitant with the gel formulation, the pain and tolerability of these procedures can be dramatically improved.

As such, methods and formulations can be used as an adjunct to any method of denervating nerves in order to prevent or minimize ensuing regeneration or to prevent or reduce neuroma formation. In addition, various formulations including those disclosed herein can be used to reduce neuritis and deafferentiation pain (sometimes known as anesthesia dolorosa), reduce the formation of aberrant neural connections whether they be “short circuits” or incorrect reflex circuits between afferent and efferent, efferent and efferent, afferent and afferent, afferent and target organ/tissue, or efferent and target organ/tissue. In particular, aberrant circuits that result propagate a dysfunctional circuit resulting chronic pain and inflammation in the tissue or end organ or referred pain elsewhere. These circuits may also result in a change in the threshold for these nerves firing and therefore methods to prevent the formation of aberrant circuits or to prevent the chronic change in firing threshold for action potentials are desirable.

Application Post-Denervation. In one embodiment, the denervation therapy can be applied or delivered prior to the administration of the formulation. For example, the denervation therapy can be applied to the site and immediately thereafter up to 2 weeks later, such as immediately after to up to 1, 2, 3, 4, 5, 6, 7, 8, 9, 10, 11, 12, 13, or 14 days later, or ranges including any two of the aforementioned values.

In some embodiments, the formulation can be delivered through the same device that the denervation therapy is applied. The formulation can be delivered through the lumen of the device regardless of if the device delivers radiofrequency, cryo, electroporation or other therapy through the tip of the needle or catheter.

Alternately, the formulation can be delivered one to three days or more after the nerves have degenerated and some of the neural and tissue debris cleared from the site. The formulation can in some cases be delivered before the formation of scar tissue as this may impede the ability of the gel to flow and spread throughout the site. Alternatively, the denervation therapy may be delivered to the target site first and then the blank gel or neuromodulatory gel can be delivered through a second delivery system. For example, noninvasive denervation therapies, such as described in U.S. Pat. No. 9,352,171 to Gartner, incorporated by reference in its entirety, can be utilized or modified for use herein. However, in some embodiments, the denervation therapy is delivered or applied with the same device that then delivers the formulation, for example, the gel.

Surgical. In one approach, the nerve can be surgically isolated and transected, as needed, prior to delivery of the formulation. This approach potentially allows for more complete mechanical disruption of the nerve bundles while also providing a potential space for the placement of the formulation (e.g. gel). Similarly the formulation can be delivered after the application of a local RF/cautery device ablation or transection of the fibers. Devices that can be utilized, for example, include the COOLCUT adiofrequency devices (Arthrex, Naples, FL).

Post-Injury/trauma. In another embodiment, such as after a compression or other traumatic injury, the region surrounding the damaged nerve(s) can be decompressed and/or cleaned up prior to the application of the formulation.

Minimally invasively. In another embodiment, the formulation can be delivered through the lumen of a radiofrequency ablation needle after performing RF ablation. One example of this would be for the treatment of lumbar facet arthritis with RF ablation of the lumbar facet joints at, for example, L1, L2, L3, L4, and/or L5.

In some embodiments, gel delivery can be combined with a lumbar facet joint nerve ablation or rhizotomy, cervical facet joint block for neck pain, third occipital nerve, peroneal nerve neurotomy or release, genicular nerve ablation, sacroiliac joint ablation, medial branch radiofrequency, or other procedure. In some embodiments, chronic osteoarthritis pain of the knee is often not effectively managed with prescribed medications. Radiofrequency (RF) ablation, when applied to articular nerve branches (genicular nerves), provides a therapeutic alternative for effective management of chronic pain associated with osteoarthritis of the knee. In some embodiments, any number of the following genicular nerve branches can be targeted, including the articular branches of various nerves, including the femoral, common peroneal, saphenous, tibial, and obturator nerves. Diagnostic genicular nerve blocks can be first performed under imaging guidance, and a local anesthetic injected, for example, arodunt eh superior lateral, superior medial, and/or inferior medial branches. A response could be positive if there is a 50%+pain reduction in 24 hours. A hydrogel including any therapeutic agent (or blanks), such as those disclosed herein can then be utilized, in conjunction with or in lieu of an genicular nerve RF ablation (GNFRA) procedure. In some embodiments, a GNRFA procedure can optionally also be performed is typically performed with local anesthetic, although deeper sedation may be offered. Either ultrasound or fluoroscopic guidance may be used. Following local anesthetic infiltration, a radiofrequency cannula is guided in proximity to the locations of each nerve. Sensory stimulation can be used to confirm accurate placement. Motor stimulation can also be performed to ensure the absence of adjacent motor fibers. After positive confirmation of sensory placement and negative motor testing, 2 mL of 2 percent lidocaine is administered adjacent to the nerve to mitigate pain associated with radiofrequency lesioning. Radiofrequency ablation is performed, for example, at a temperature of 70° C. for about 90 seconds. A hydrogel can then be delivered to the genicular nerves as described elsewhere herein.

In some embodiments, RF ablation can be performed using a hollow needle-based device and subsequent to the thermal ablation, the hydrogel is injected into the lesion site. In another embodiment, cryotherapy ablation, such as with the IOVERA (Myoscience, Fremont, CA) device. A longer lasting effect can be achieved by depositing some regeneration-blocking gel at the ablation site. In particular, the IOVERA device can be adapted to deliver nitrous oxide through one lumen and the hydrogel through another lumen, possibly a central lumen. These energy modalities in combination with a blank or drug-loaded gel may be delivered percutaneously, such as through a needle or needle-shaped device, through a direct surgical approach, or through an endovascular approach.

In some embodiments, cryotherapy such as liquid nitrogen can be utilized, including. freezing for a period of time, and repeating as necessary. Other non-limiting devices that can be utilized include the Focal Cryo Catheter (Medtronic, Dublin, Ireland), truFreeze Spray Cryotherapy (CSA Medical, Lexington, MA), C2 CryoBalloon, Pentax Medical, Redwood City, CA) the Arctic Front Cryoablation (Medtronic, Dublin, Ireland), and the HIMG cryoablation catheter system (Medtronic, Dublin, Ireland), Cryotherapy can be applied in some cases utilizing an 18 gauge needle with a cryotherapy gun. In some embodiments, the cryotherapy is delivered immediately prior to the delivery of the neuromodulatory or blank hydrogel.

In some embodiments, hydrocission is utilized to dissect or transect the target nerves prior to delivery of the neuromodulatory gel. Hydrocission has been used as a method to ablate tissue such as the prostate (Aquabeam) or the spinal/discectomy procedures (Hydrocisions, Inc Spineject Microresector Arthroscope). Hydrocission may be employed using a needle or catheter delivered to the nerve location using a high-velocity saline stream to transect the nerves but not the surrounding blood vessels. The procedure can be performed under image or robotic guidance. The hydrocission technique can be used on any of the nerves but may be particularly appropriate for nerves that are entrapped is scar tissue.

In some embodiments, biopolar or unipolar electrocautery is used to ablate the nerve prior to delivery of the hydrogel.

In some embodiments, a blank gel or slurry including others as disclosed herein can be injected in and around a nerve in order to block nerve regeneration. The gel or formulation provides an in situ physical barrier to gel regeneration akin to the use of cheesecloth or sutures to prevent nerve regeneration. The solution can be injected at one viscosity, and then, upon forming a gel, is no longer flowable and conforms to the injection site. The gel may have characteristics that allow it to adhere to the nerve and surrounding tissue and remain in place for a period of one to 6 months or more, more preferably 2 to 3 months. If the gel is applied in excess to otherwise healthy neural tissue, it is biocompatible and does not disrupt the function of off-target neurons.

In some embodiments, a blank gel or neuromodulator gel may be delivered around a nerve percutaneously to prevent or reduce nerve entrapment. The hydrogel can be delivered around the nerve, for example, nerves that have become entrapped, in combination with an anesthetic and an NSAID to reduce the nerve inflammation and then to provide a barrier for ongoing inflammatory cell migration to the nerve. The hydrogel can also provide a gliding function so that the nerve may more freely glide again within its fascial tunnel. In one embodiment, in situ forming hydrogels are delivered to the nerves in the carpel tunnel to treat nerve entrapment syndromes there. In another embodiment the hydrogel is delivered around the ulnar nerve percutaneously to reduce to inflammation around the elbow.

Adding anti-growth permissive drugs. A gel barrier may be further reinforced with the incorporation of anti-growth permissive drugs (e.g., myelin basic protein), by reducing the pore size of the hydrogel thereby preventing or delaying large molecular diffusion through the matrix (e.g. growth or neurotrophic factors).

Conjugating anti-growth permissive drugs. Alternatively, inhibitory peptides or molecules can be conjugated to the backbone or side chains of the gels in order to further prevent neurite sprouting and attempted regeneration.

Direct surgical exposure. In one embodiment, the nerve can be directly exposed surgically through a minimally invasive or open surgical approach. After the nerve is transected or isolated, the in situ crosslinking gel can be injected around the nerve to form a crosslinked gel. The gel is flexible but also sufficiently rigid that it will prevent nerve sprouting and regeneration into the gel and thus reduce or prevent the formation of neuromas.

Neuromas. In the case of neuromas in load-bearing locations, the blank gel may be injected in a volume sufficient that the gel acts as a temporary padding to reduce the pressure on the neuroma itself.

In some embodiments, such as for applications in the periphery, the nerve can be injected within the epineurium with the in situ forming gel solution after delivery of a cryo-thermal- or chemolytic agent to the site in order to block regeneration from one of the neuroablative therapies. The gel can be configured such as to spread a distance within the epineurium and then form an impenetrable barrier to nerve regeneration for 0 to 12 months, more preferably 0 to 3 months. In some embodiments, as desired, the in situ crosslinking gel can be nonbiodegradable, forming a permanent implant at the injection site. In other embodiments, the number of hydrolytically or enzymatically cleavable bonds can be reduced such that the gel, while degradable, degrades over one to two years.

Examples of peripheral nerves that would be desirable to treat with the gel include the sciatic, peroneal, or tibial nerve, or others including those disclosed elsewhere herein. These nerves are sufficiently large enough that a 22 to 24 gauge needle can penetrate the epineureal sheath and deliver agent within the nerve bundle to prevent or reduce nerve regeneration.

In addition, in some instances it is desirable to protect nerves that have been damaged through transection, crush, or other injuries from neuroma formation. In these instances, the gels can be injected to the site of the injury through a needleoscopic/needle-based approach or

In combination with other therapies. Although the blank gel can be delivered after the RF or cryotherapy has been injected or administered to the site, it is more desirable to deliver the gel immediately subsequently to the neuroablative therapy, preferably utilizing a combined system that can deliver the RF or cryo energy followed by the blank or neurolytic agent loaded hydrogel.

Other alternatives. Alternatively, if the nerve has been damaged, the blank hydrogel can permit regeneration within the damaged epineurium but prevent off-target innervation or the formation of painful neuromas. Alternatively, if the nerve has been damaged through an injury, the gel can be delivered into the epineurium or around the nerve to reduce neuroma formation.

Hydrogel Delivery after RF Ablation of Sympathetic Chain

In some embodiments it may be desirable to deliver the gel, with or without neuromodulatory agent, after the delivery of radiofrequency (RF) or other thermal energy modality to the paravertebral gutter. There are numerous case reports and even some clinical series in which patients with hyperhydrosis, causalgia/reflex sympathetic dystrophy, Raynauld's disease, heart failure and angina are treated with percutaneous RF sympathectomy which is performed with multiple single point ablations at each level of the thoracic chain. There are no anatomic landmarks to confirm the needle placement against the sympathetic chain. The efficacy of this approach can be suboptimal due to incomplete ablation and regeneration of fibers within 3 to 12 months, frequently within 6 months. Therefore, it may be desirable to improve the efficacy of the procedure by delivering a gel, slurry, or semi-solid formulation after the thermoablative procedure. The gel, with or without neuromodulatory agent, may further facilitate more complete neuronal cell death and prevent regeneration that occurs after RF lesions. In one embodiment, RF lesions are performed with a needle-based approach at between 6° and 90° C., preferably 70° C., for between 30 and 120 seconds, preferably 90 seconds per lesion. Due to the large size of the stellate and other ganglia, multiple RF lesions are typically required and the hydrogel can be injected through the monopolar RF catheter after the lesion is created. Alternatively, the gel can be delivered prior to the RF lesion in order to facilitate the thermal spread.

Neuromodulation approaches to block, temporarily block, or eliminate nerves include, for example, nerve blockade or blocking, neuroablation, chemoablation, radiofrequency ablation, neurolysis, chemolysis, chemical neuroablation, such as sympathicolysis. Alternatively, when a procedure to remove nerves is performed it may involve the following terms-icotomy, -ectomy etc. such as sympathectomy or sympathicotomy or ramisectomy. Neuromodulation can refer to chemical, mechanical, or electrical methods to modulate nerve conduction. Electrical methods include hyperpolarization block, cathodal, anodal, or collision block, as described for example in U.S. Pub. No. 2005/0228460 A1 to Levin et al., which is hereby incorporated by reference in its entirety. Overpacing or overstimulating a nerve (such as with a neurotransmitter, e.g. nicotine) may also induce a block by generating stimulating the nerve at a rate that exceeds its capacity to generate an action potential. In this manner, neurotransmitters stores are depleted and the nerve is temporarily unable to convey signals to other nerves and tissue.

The radiofrequency generator and catheter can have, in some embodiments a closed loop element to measure impedance, deliver electrical stimulation (e.g. 2 Hz current for motor and 50 Hz for sensory) to confirm the position of the electrode in the location of the paravertebral gutter as well as deliver ablative energy to arbitrarily or selectively interrupt the sympathetic chain. In one embodiment, sensory stimulation is carried out at 50 Hz until a tingling sensation in the select dermatome noticed using a 0.4 to 1 V stimulus. It may also be possible to perceive muscle contractions (anterior root) at a threshold 1.5 times below the sensory threshold. In addition, the system can perform thermal lesioning with a preferably bipolar electrode with a continuous and/or pulsed RF signal, and monitor the temperature at the tip of the device. In one embodiment, the RF energy is continuous, providing indiscriminate destruction of sympathetic afferents and efferents. Alternatively, the parameters of pulsed radiofrequency signal allow the targeted ablation of C-fibers. In one embodiment, a 100 mm cannula (Radionics SMK 22G, 5 mm active tip) is inserted and advanced to the paravertebral space. The stylet of the cannula is replaced with a flexible RF probe with either a monopolar electrode at the tip or bipolar electrodes spaced, for example, 1-10 mm apart which is advanced through the paravertebral space up to the desired thoracic level, for example from T4 up to T1, or up to the middle of the first rib. Alternatively, the catheter can be advanced rostrally one or two levels, the energy delivered, and then retracted and directed caudally where the catheter then delivers RF at one or two levels.

In one embodiment, contrast is injected (e.g. iohexol) to confirm the appropriate positioning of the cannula and confirm an extravascular location and absence of intrathecal or intrapleural spread followed by 2 ml of 2% lidocaine. The hydrogel formulation is then injected at the desired target levels. The hydrogel, such as the in situ crosslinked PEG-based hydrogel, has enough mechanical integrity to allow a catheter to move through them without collapsing. A flexible RF probe with a thermocouple electrode is advanced to the target region through the hydrogel. In this case, the hydrogel has created a potential space through which to deliver the RF energy with less concern for inadvertent perforation of the pleura. The RF probe may utilize a local cooling mechanism, such as a balloon, to prevent local destruction of the hydrogel where the highest temperatures are present (e.g. melting). Destruction of the ganglia and nerves within the paravertebral gutter occurs between 60° C.-up to 75° C. or more.

In another embodiment, saline (with or without contrast) is delivered through a catheter to fill the paravertebral gutter and to push the pleural membrane away from the sympathetic chain. The steerable RF catheter is advanced up or down the paravertebral gutter and energy delivered at appropriate intervals to destroy the sympathetic chain, sympathetic ganglia, and rami communicants. In another embodiment, the advancing front of the RF catheter has a saline port that allows for a moving from of expansion of the pleura to create the potential space within the paravertebral gutter through which the catheter can advance safely. In another embodiment, the RF catheter has side ports that continuously deliver saline around the catheter tip and shaft to allow it to advance safely within the paravertebral gutter.

The probe may include continuous or overlapping probes (4 to 10 mm probe approximately 10 mm apart), both of which, with the appropriate spacing, can generate a contiguous lesion. Burns can then be performed in approximately 2-5 minutes with a cooling catheter. Radiofrequency is delivered at a frequency of 500 kHz and a power of 1-50 Watts for a defined period of time, for example 1-30 minutes, preferably 1 to 5 minutes. Several embodiments using low-dose radiofrequency ablation, such as those delivered to ablate target nerves within and alongside the adventitia of the renal arteries to treat hypertension, may be applicable. For example, ramped low power RF energy (5 to 8 watts) for a select period of time (2 minutes) may be desirable (as disclosed, for example, in U.S. Pub. No. 2011/0207758 A1 which is hereby incorporated by reference in its entirety). Various embodiments of methods, apparatuses, and systems for performing renal nerve ablation are described in greater detail in U.S. patent application Ser. No. 12/545,648, filed Aug. 21, 2009 and PCT/US09/69334, filed Dec. 22, 2009, both of which are incorporated herein by reference in their entireties. In one embodiment the energy is delivered circumferentially and in another embodiment the electrodes and the catheter can be biased to direct the energy in one direction, such as posteriorly.

Cryotherapy In another embodiment, a cryoballoon or probe containing internally circulating liquid nitrogen can be similarly delivered within the paravertebral gutter as the RF system embodiments, except that the temperature between 0 to −200° C. (e.g. adaptation of CardioCryo technology).

Microwave energy delivered in the range of 0.9 to 2.4 GHz at a power of 1-100 Watts applied through monopole, dipole, half-dipole, or helical coil antenna.

Ultrasound. The ultrasound transducer may range from 0.1 to 10 mm, more preferably 0.1 to 3 mm, more preferably less than 1 mm. At this range, frequencies from 2 MHz to 15 MHz may be employed, more preferably 5 to 10 MHZ, more preferably 7 MHz. Using this range of frequencies, the transducer may be focused at a distance less than 25 mm, preferably less than 10 mm, more preferably less than 5 mm, more preferably 2-3 mm away. Overall transducer surface area may be 1 to 50 mm², more preferably 12 mm². Acoustic power may be around 1 Watt. The transducer may be a cylindrical transducer curved about its longer dimension, a cylindrical transducer curved about its shorter dimension, a concave transducer curved along the shorter dimension, or a flat transducer. An ultrasound transducer may also be employed that uses an inflatable membrane and solid diffraction lens. Multiple transducers with different focal lengths may be employed.

Minimally Invasive optics. In some embodiments optical fibers with a light source for illumination of the paravertebral gutter may be employed with these devices.

Catheter based transmural RF delivery. In one embodiment, a balloon flexibly and irregularly expands to conform to the vessel wall, such as a vein, particularly in regions where the vein is bifurcating and the ostia are irregularly shaped and sized. The balloon has cooling fluid in it to protect the vessel wall and an atraumatic tip on the balloon guide avoids damage to the vessel or trauma to the pleura.

Electromagnetic. Paramagnetic nanoparticles or microparticles can be delivered to the region surrounding the sympathetic chain and then heated with an external electromagnet to ablate the nerves (ApexNano Therapeutics, Israel).

Insulated tips. A steerable cannula with a tapered tip can be placed at the target location. The cannula can have smooth tapered siliconized or non-siliconized insulation with tip exposures of 2, 4, 5, and 10 mm, or up to 20 mm. The tip may be straight, curved, and sharp, or blunt.

Nerve tissue may be decreased, partially or completely destroyed or removed, denervated deactivated, or down-regulated. Nerve tissue may undergo necrosis, apoptosis, atrophy, gene expression down or up regulation, protein expression down or up regulation, ablation. With some approaches, the entire nerve may be destroyed and in other approaches, specific regions of the nerves may be targeted such as the ganglia or soma, the axons, and/or the nerve terminals.

Intravascular Device Energy Modalities. In some embodiments, the neuromodulation may be achieved from an intra- to extravascular approach via an endovascularly placed device in proximity to the target neural matter or through a percutaneous paravertebral approach. In some embodiments, the therapy can be delivered from an anterior approach directed towards the stellate ganglion and the top of the TPGS. Alternatively, an anterior approach similar to that of the approach to the stellate ganglion but with subsequent guidance-based navigation to the top of the paravertebral gutter, may be desired. In this manner, the therapy can be delivered from the top of R1 down to the desired target level directly. In some cases, the therapy is delivered from one injection site to multiple paravertebral levels, in contrast to other approaches that ablate only one level at a time, such as described, for example, in U.S. Pub. No. 2013/0296646 to Barbut et al., which is hereby incorporated by reference in its entirety.

Generally, the neural modulation may be achieved in some embodiments through electrical, thermal (heating or cooling; resistive or infrared), mechanical, or chemical energy. The energy delivery device can be located in or otherwise associated with the catheter and emits energy from the catheter. The energy delivery device may be located in a needle tip and energy is emitted from the needle in some cases. An energy delivery device such as, but not limited to, high voltage field pulses, direct current, monopolar radiofrequency (such as described, for example, in U.S. Pat. No. 8,175,711 to Demerais et al., incorporated by reference herein in its entirety), bipolar radiofrequency, pulsed radiofrequency, high-intensity focused ultrasound (HIFU) (continuous, pulsed) (such as described, for example, in U.S. Pat. No. 8,206,299 to Foley et al., incorporated by reference herein in its entirety), low-intensity focused ultrasound (LIFU), nonfocused ultrasound, other forms of ultrasound, microwave, laser, steam or hot water, cold radiation, cyrotherapy, optical, light, phototherapy, X-ray or radiation therapy (such as described, for example, in U.S. Pub. No. 2013/0035682 to Weil et al., which is hereby incorporated by reference in its entirety) magnetic, electromagnetic, plasma, reversible or irreversible electroporation, lithotripsy (extracorporeal, intracorporeal, extracorporeal shockwave therapy), vibrotactile, kinetic, potential, pressure, nuclear, elastic, and/or hydrodynamic energy (as outlined, for example, in U.S. Pub. No. 2013/0204068 to Gnanashanmugam et al., which is hereby incorporated by reference in its entirety). The mechanical device may perform, for example, cutting, sealing, ligation, or clipping.

These energy modalities can be delivered in conjunction with a blank gel or hydrogel if desired to improve the conduction or distribution of the electro-thermo- or mechanical signal. By way of example, delivering a gel to the target regions within the paravertebral gutter, for example, can allow for expansion of the paravertebral gutter space and subsequent delivery of a monopolar or bipolar RF catheter after insertion at one paravertebral level (e.g., endovascularly, transcutaneously) to the region without disruption of the pleura. The temperature at the target tissue should be 45° C. or more in some embodiments to cause irreversible damage to the neuron. Higher temperatures exceeding 90° C. may cause boiling of the tissue. The generally agreed upon target temperature for some embodiments is between 60 to 90° C. for about 90 seconds. By delivering an anesthetic in the gel, local pain relief can be achieved prior to the application of RF energy. Subsequently, the energy delivery can be achieved in a more uniform manner the sympathetic chain. In another embodiment, a ‘blank’ hydrogel is delivered and the properties of the hydrogel alone temporarily stun or block or permanently destroy the nerves within the paravertebral gutter. The hydrogel delivered may be acutely toxic to nerves, such as some of the Pluronic formulations, resulting in local cytotoxicity and nerve death. Alternatively, excipients, such as generally recognized as safe (GRAS) excipients may cause changes in ion fluxes in the neurons resulting in neuronal excitotoxic cell death is triggered. By way of example, the delivery of glutamate to the neurons may trigger local nerve degeneration. In another embodiment, the ‘blank’ hydrogel swells slightly as it equilibrates with the host tissue resulting in a compressive injury to the nerves within the paravertebral gutter, particularly since they are adherent to the vertebral body or rib. In another embodiment, a ‘blank’ gel (e.g., including galvanic alloy particles) undergoes an exothermic reaction and releases heat as it gels within the paravertebral gutter to destroy nerves. In another embodiment the gel is heated prior to its deposition in the paravertebral gutter, whether at the tip of the catheter delivery system or extracorporeally. In another embodiment it is a cryogel, and is injected in a cooled state, resulting in temporary or short-term block to the nerve fibers in the paravertebral gutter. In another embodiment, to overcome the challenges of advancing a catheter in the paravertebral gutter, an RF catheter has a port on the front or front sides of the catheter for delivering an advancing front of gel in front of the RF catheter to protect the pleura from inadvertent puncture. In this manner a catheter delivering mechanical or thermal energy can ablate multiple levels of nerves within the paravertebral gutter.

### Drug Delivery Systems and Catheters to Deliver Those Gels.

Control the spread. Sympathetic chain neurolysis is performed on a very limited basis, typically in patients suffering from severe intractable pain in late stage cancer in which the benefits in pain reduction out way the risks. Paravertebral or stellate ganglion injections of alcohol or phenol are considered risky because of the adverse events that occur in some patients as a result of their unintended spread (as described, for example, in U.S. Pat. No. 8,211,017 to Foley et al., which is hereby incorporated by reference in its entirety). Along the sympathetic chain this can occur at 1) the cervical levels in which the neuromodulatory agent may spread to the recurrent laryngeal nerve, the vagus, or the brachial plexus (as is often observed with large volume anesthetic stellate ganglion blocks) resulting in adverse events and 2) paravertebral levels (thoracic, lumbar) in which the agent may spread to the intercostal nerves or through the intervertebral foramen to the epidural space causing temporary or permanent neuritis, neuralgia, and even rarely paraplegia. Therefore, formulations that can deliver a neurolytic agent into the paravertebral gutter while preventing the uncontrolled spread of the agent to off-target structures, particularly neural structures including the spinal cord, can be desirable. In one embodiment, the spread of the neurolytic is limited through the injection of the neurolytic in a biocompatible drug delivery system, preferably a gel. In this manner, the spread of the drug is determined initially to the location of the where the formulation is deposited and ultimately resides and subsequently the drug is reaches adjacent tissue through diffusion.

Single level. Furthermore, the uncontrolled spread of neurolytic agent has limited the use of a one-level injection therapy to reach multiple dermatomes or levels. As a result, many clinicians use more than one injection sites at multiple levels to achieve a continuous paravertebral block. By loading the neurolytic agent into a gel, the improved control of the spread of the neurolytic agent will also improve the longitudinal spread within the paravertebral gutter.

Local delivery. Another objective of delivery of neurolytic agents to the sympathetic chain or nervous system, generally, is to achieve a more consistent, complete and reliable denervation. Recent clinical studies have demonstrated that more complete denervation of the sympathetic fibers coursing around the renal artery is linked to improved and more consistent outcomes in the treatment of hypertension. By developing a more controlled delivery and more complete ablation, patients may elect the procedure if it has demonstrated to be safe, if the responder rate (efficacy) is high, and the efficacy is consistent. Denervation of the sympathetic chain is currently performed through a minimally invasive surgical procedure (VATS) on a limited basis for the treatment of complex regional pain syndrome or hyperhidrosis. Several papers have demonstrated that more complete denervation results in better efficacy in these patients. Sympathectomy (transection and removal of chain), for example, has superior efficacy and durability than sympathicotomy (in which the nerves are cut but the chain is not removed) or clipping of the chain. Percutaneous approaches have been limited to single-level RF ablation which is widely recognized to result in incomplete chain ablation due to the inability to precisely localize the sympathetic chain, resulting in only partial denervation.

There are only a handful of open-label limited patient studies exploring neurolytic ablation (alcohol, phenol) of the sympathetic chain, such as the stellate ganglia, for the treatment of other indications. In these cases, the desire to achieve effective denervation of the target tissue is achieved through the injection of large volumes of neurolytic agent in and around the target neural tissue. Presumably, the goal is to bathe the tissue in the neurolytic agent for as long as possible and to ensure complete coverage of the target tissue. Again, this high-volume high-spread solution comes at the expense of safety, thereby limiting the applications of neurolytic agents to medically refractory cases. Thus, a localized drug delivery system that delivers the neurolytic agent to the target neurons with minimal disruption to adjacent non-target neural structures can be desirable. In one embodiment, this can be achieved with a formulation in which the drug is delivered within a gel. In this embodiment the boundaries of the gel determine the deposition of the drug and the subsequent zone of diffusion of the drug out of the gel to the target tissue down the concentration gradient. In this embodiment, a smaller volume of neurolytic gel can be delivered to the patient than is delivered with an injection of a neurolytic alone.

Sustained release. In the case of the administration of a neurolytic agent, it is desirable that the agent be released from the drug delivery system in some embodiments from, for example, 12 hours to four weeks, 1 day to 1 week, or 1 day to 3 days. The drug delivery system, in some cases, affords higher loading levels of drug with reduced systemic toxicity than can be achieved with the drug alone. High local concentrations of the drug can be delivered in a sustained way such that although the drug is delivered above its therapeutic window, in the local cytotoxic range, the burst or spike in concentration that occurs after the delivery of the drug in a bolus can be reduced, and a more typical local sustained release profile can be achieved. A sustained release therapy for the treatment of cardiac arrhythmias, particularly ventricular arrhythmias though the denervation of, e.g., the T1-T4 sympathetic chain can be desirable.

Controlled release. In some embodiments, it can be desirable to deliver one, two, or more agents within its local therapeutic window for a longer period of time, such as with neuromodulatory agents. In one embodiment, these agents are delivered locally within their therapeutic window, in some cases without a burst phase in which the agents reach supraphysiologic/toxic levels. Again, with a higher drug loading level than can be achieved with a drug solution alone, sustained or controlled release of drug can be achieved, for example, for 1 day to 9 months, 1 week to 6 months, or 2 weeks to 4 months. In one embodiment, agents that can provide a ‘chemical sympathectomy’, such as reserpine, can be delivered locally to the cardiopulmonary sympathetic nerves to decrease the release of epinephrine and other neurotransmitters without destroying the sympathetic nerves themselves. Similarly, the durability of nerve block with sympathetic agents can be extended with a controlled release hydrogel. In one embodiment, the long duration treatment of angina can be desirable without denervating the nerves. In this manner, reserpine can be delivered through a controlled release drug delivery system for, e.g., the 3 month to 6 month or more treatment of refractory angina. Patients that undergo a temporary block, may escalate their treatment to a longer-duration temporary block and from there to the permanent neurolytic block if the other blocks are providing adequate relief.

Release rate. The release of the drug may be diffusion controlled, chemically or biodegradably controlled, solubility controlled (of the drug), solvent controlled (swelling, osmosis, rupture), or externally activated/modulated (e.g. magnetic system in which micromovement of magnetic beads within a hydrogel causes movement and thus drug release, low frequency ultrasound, electroporation), controlled by the extent of crosslinking and crystallinity, the size, thickness or volume of the drug delivery system, the porosity, and the solubility of the system (e.g. plasticizers or the additional of hydrophilic agents (e.g. glucose, mannitol)) that are rapidly dissolved and create a network or pathway for dissolution of the drug out of the system, or controlled by the degradation of the hydrogel scaffold. The release rate of the drug may also be controlled by the pH, ionic strength, temperature, magnetic field, ultrasound, or electrical stimulation. Preferably, the release of the agent is not controlled by the degradation of the polymer. The release rate may be monomodal, bimodal or polymodal. The release rate may include a burst phase and then a linear continuous sustained release phase. The solubility of the drug in the aqueous phase drives the rate of drug release with poorly water soluble drugs providing longer release than the higher solubility drugs.

Blank hydrogel. Another approach is to deliver a blank hydrogel to the site, defined as a hydrogel without any active pharmaceutical ingredient (API). The hydrogel may contain a neurotoxic solvent, such as ethanol, DMSO, propylene glycol, glycerine, glycerol, D-Limonene, methanol, ethanol, octanoic acid, 2-octanone, diethyl ether, benzyl alcohol or a preservative such as thimerosal or chlorbutanol. The hydrogel or crosslinking agents, the pH of the injectate, the pH of the formed hydrogel, the temperature liberated as a result of the gel crosslinking, or the change in the extracellular ion concentrations may cause local neurotoxicity in the absence of an API.

Expanding or Conformal Filling the potential space. Delivering the solution in a suitably viscous formulation, such as a hydrogel, slurry, an injectable foam, a glue or an in situ forming injectable scaffold, including a hydrogel, slurry, semi-solid, spray, or other gel that can fill the majority of, or substantially the fascial space or fascial plane containing the nerves, such as via a 17 gauge needle or higher in some cases. Some examples of slurries that can be used with embodiments disclosed herein can be found, for example, in U.S. Pat. No. 7,057,019 to Pathak, which is hereby incorporated by reference in its entirety. In one embodiment, the therapy is a viscous solution or gel that can be injected with a minimally invasive technique to fill an anatomical space and adheres to the edges of the tissue. In filling the paravertebral gutter, a more complete acute denervation of the nerves in the paravertebral gutter as the agent is delivered in and around all structures within the gutter. This conformal filling of the potential space can be performed with a radiopaque or echogenic polymer.

Preventing reinnervation. Approximately 5-15% of surgical have chronic pain or late recurrence of pain that is thought to be due to reinnervation. Specifically, they are attributed to incomplete resection or ablation of sensory pathways with 1) remaining residual sensory connections that may be strengthened by reinnervation 2) regeneration or surviving neurons and 3) the formation of alternate sensory pathways resulting from sprouting from the same nere stump more proximal (close to the cell body) to reinnervate the distal target tissue. By delivering the hydrogel to fill the potential space surrounding the nerve, more proximal sprouting from health nerve away from the lesion site can also be prevented with a physical barrier downstream. These sprouting fibers will face the same insurmountable obstacle. One approach to preventing regeneration is to deliver a neuroinhibitory formulation to block regeneration through a lesion site. In one embodiment, the biodegradable or bioresorbable hydrogel maintains its integrity for the duration of attempted regeneration but is completely degraded or resorbed by the patient's body thereafter. More specifically, the bulk of the hydrogel degradation occurs after 2 to 6 months, more preferably 3 months or more, more preferably 4 months or more. Prior to degradation, the mechanical integrity of the hydrogel is critical to maintaining the barrier. The hydrogel must be free of cracks of fissures through which the nerves could cross, must be flexible and able to withstand cyclical loading exerted by the surrounding muscles during use of the limb. In this case, the lesion site is the region that was lesioned by the neurolytic agent, the presence of a non-growth permissive hydrogel at the site prevents the formation of appropriate connections between remaining nerve fibers and thus prevents appropriate or inappropriate reinnervation of the target tissues. Similarly, without supporting glia in the matrix, such as Schwann cells, and the retardation or prevention of trophic factor diffusion through the gel the absence of trophic support will provide an additional barrier to axon outgrowth and subsequent reinnervation. Thus, while neurons initially extend axon growth cones, an adverse environment will result in dispersion of these nerve sprouts and ultimately aborted sprouting. At a clinical level, this may translate to fewer late therapy failures as a result of regeneration and or incomplete denervation resulting in faster reinnervation.

Porosity. Controlling the pore size of the gel provides another mechanism to control the release of drugs, particularly low molecular weight drugs, as well as to prevent cellular infiltration or axonal regeneration within or across the hydrogel. In some embodiments, the gels can have a pore size of less than 50 μm, 20 μm, 10 μm, or even less. These gels can be non-porous or minimally porous for a period of time (e.g., 2-3 months) until the polymer beings to degrade. In some embodiments, the pores are too small for Schwann or immune cell ingrowth (e.g., less than 8 μm), and the density of pores is not such that a network is formed between the pores. In one embodiment, the use of low MW polymer chains between crosslinks reduces the chain flexibility, reduces mesh size/pore size, and convers an advantage to delay the release of drugs out of the gel. In one embodiment, small pores (<8 μm) assist with the echogenicity of the hydrogel but are smaller than infiltrative cells such as Schwann cells, other supporting cells, immune cells and axons. In still another embodiment, the pores are microporous (e.g., from about 100-500 Angstroms). Some examples of hydrogels with pores can be found, for example, in U.S. Pat. No. 8,399,443 to Seward, which is hereby incorporated by reference in its entirety.

The inability of cells to grow into the scaffold can be maintained in some cases for one to 6 months, such as one to two or three months, during which the damaged nerves are attempting to regenerate to targets on the other side of the gel. After this, the degradation of the polymer can result in cellular ingrowth.

The porosity, and thus the cellular ingrowth, can be controlled with either the the concentration of the multi-arm PEG (solid content %) or the crosslinking density (controlled by pH, gelation time, molecular weight and number of PEG arms) and thus the stiffness of the hydrogel. By increasing the stiffness of the gel or the modulus of elasticity, the extent of cellular invasion and migration can be further reduced. In one example, vinyl sulfone derivatized 4-arm PEG (20 kDa mW) was prepared by reacting the derivatized PEG with equimolar ratios of vinyl sulfone groups on the PEG and thiols of cysteine residues (e.g., not the bis-cystein MMP sensitive peptides). Gels of between 1 and 5% m/v were prepared. These gels can be injected and polymerize to yield a well-defined porosity of around 82% with 157 micron pores. In another embodiment, the porosity of the hydrogel is less than 30%, more preferably less than 20%.

In one embodiment, polymers with small pore or mesh sizes act as the rate-limiting factor in diffusion of drug out of the hydrogel. By controlling the pore size to less than 5 microns, or more preferably less than 1 micron, for example, a small molecule may diffuse out of the scaffold but cells such as axons, glia and inflammatory cells cannot enter the scaffold, inhibiting any functional reinnervation. Pore size can be varied with the degree of crosslinking and the molecular weight of the crosslinks of the gel.

In another embodiment, the pore size of the hydrogel can be controlled to prevent axon ingrowth with pores less than about 50 microns, 20 microns, or 10 microns. Alternatively, the scaffold pores are not interconnected within the matrix. Alternatively, the pores are not oriented in such a way to promote the extension of cells into the scaffold. For example, scaffolds without pores may not encourage axonal ingrowth.

Porous fibrous scaffolds, such as the self-assembling peptide hydrogel matrix, PuraMatrix, are less desirable in some cases since the polymerization results in a nanometer scale loose fibrous structure that is designed to encourage cell infiltration and growth within the scaffold. These scaffolds have been demonstrated to encourage attachment and outgrowth of neuronal cells, features that would not be suitable for providing a physical barrier to nerve regeneration. Some self-assembling peptide hydrogels are disclosed, for example in U.S. Pat. Nos. 8,465,752, 9,011,879, and 9,199,065 as well as U.S. Pub. No. 2011/0104061 and 2013/0287698, all of which are incorporated by reference in their entireties. However, in some embodiments, these hydrogels may not be suitable for this application given the high rate of cellular ingrowth.

Bioadhesive. The hydrogel can be designed, in some cases, to covalently or noncovalently, ionically or nonionically, adhere to the adjacent tissue particularly that of epineureum and nerve cells. Direct apposition between the hydrogel and the nerve target permits improved targeting of drugs to the site. Improved adhesion also reduces the physical space permitted for any nerve sprouting and prevents and or blocks the formation of neuromas after percutaneous nerve ablation. In one embodiment, the hydrogel adheres directly to the nerves that it is surrounding through crosslinking with neural tissue. In one embodiment, cationic interactions improve the adhesion of a hydrogel to the tissue. Assuming good adhesion to this tissue, there will be very little, if any path for the regenerating neurons below and above the gel to travel. Similarly, if there is good adhesion to not only the nerves but the surrounding muscle, fascial and vasculature, the space permitted for nerves to grow around the hydrogel depot is minimal.

Neuroinhibitory gels. The goal of the majority of polymeric scaffolds in development is biocompatibility, the reduction of further neural damage, the prevention of scar tissue formation and encouragement of regrowth into and through the scaffold after an injury by either modifying the scaffold or changing the agent delivered. In some embodiments, the scaffolds are designed to do the opposite: to fill the cavity in order to prevent or inhibit regeneration of nerves across the lesion zone. After nerve damage, surviving axons for growth cones and sprout into the lesion site in an attempt to reinnervate their target, typically in search of growth-factor mediated guidance cues. This sprouting occurs for a finite period of time before the regenerative attempts are aborted. In some embodiments, it can be desirable to inhibit the reinnervation of these neurons through the delivery of a gel alone or a gel loaded with neuro-inhibitory drugs, such as inhibitory peptides or extracellular matrix, to physically and/or chemically block the extension of neurites into and preferably around the gel.

In one embodiment, ingrowth into a hydrogel is inhibited by controlling the charge of the functional groups in the polymer. A neural or negatively charged polymer typically is non- or less permissive to axonal ingrowth while a positively charged hydrogel encourages ingrowth, promote tissue infiltration and axon regeneration.

In another embodiment, the hydrogel is designed that it provides a stable barrier to neurite outgrowth during the initial phase when axonal sprouting in response to injury is maximal. In this embodiment, the scaffold remains in place for 3 months without more than e.g. 50% mass loss, preferably no more than 40% mass loss, preferably no more than 30% mass loss. In hydrogels, although there may be mass loss, the mechanical integrity of the hydrogel may not be significantly affected. Therefore it is desirable to maintain the compressive strength of the hydrogel for at least 3 months after delivery of the hydrogel to no more than 50% loss of compressive strength, preferably no more than 40% loss, preferably no more than 20% loss of compressive strength. Thus, the hydrogels reside at the site of injection without fractures or creation of any other voids through which nerves could regenerate. The hydrogels particularly suited for this applications contain more biostable and less hydrolytically sensitive urethane bonds, such as PEG containing a carbonate/carbamate bonds after cross-linking. In particular, multi-arm PEGs which contain a carbamate bond after cross-linking with, for example, a PEG-amine or a PEG-trilysine are well suited for applications in which providing a barrier to nerve regeneration are desired. Commercially available systems utilizing more hydrolytically sensitive PEG-SS, PEG-SG, PEG-SAZ, PEG-SAP may provide sufficient mechanical strength for two to 8 weeks but do not provide the mechanical strength necessary to prevent nerve regeneration over the time period during which nerves are attempting to regenerate. For example, in these locations, PEG-SS, PEG-SAZ and PEG-SAP hydrogels have suffered significant loss of mechanical properties and/or are cleared within days, weeks, or a month after delivery around a nerve.

Location of the ablation along nerve. Ablation of the sympathetic ganglia and intermediate ganglia result in the destruction of the post-ganglionic sympathetic cell bodies innervating the target organ as opposed to distal ablation and subsequent rapid regeneration. Similarly, although the afferent and pre-ganglionic nerve cell bodies are not removed, their axons are destroyed close to their cell bodies in the dorsal root ganglion and sympathetic chain, respectively, resulting in less regenerative potential than if the axons were destroyed peripherally closer to their nerve terminal.

Pleural sealant. Although the rate of inadvertent pleural puncture or pneumothorax is low, the resulting adverse events, requirement for an indwelling catheter, and significantly extended length of stay in a hospital make this one of the top adverse events that clinicians worry about with paravertebral anesthetic blocks. In one embodiment, a hydrogel or therapy that can act as a tissue or pleural sealant to seal any inadvertent pleural puncture is desirable.

Echogenicity. In one embodiment, the hydrogel is naturally echogenic, such that its injection and spread is visible under ultrasound guidance. In another embodiment, an agent or microbubbles or some either echogenic component is added to the hydrogel to improve its echogenicity. In some embodiments, the combination of the neuromodulatory agent and the hydrogel improves the echogenicity and/or allows the hydrogel to be visualized under color Doppler.

Swelling. In some embodiments, the drug delivery systems undergo less than about 10%, 5%, or substantially no swelling at all when placed in situ for safety reasons.

A bioerodible drug delivery system that can control the spread of a low-molecular weight neuromodulatory drug over a period of days or months, that has the appropriate rheological and mechanical characteristics to permit the hydrogel spread within the gutter and reduce the off-target spread, provide a non-permissive substrate for neuronal outgrowth and a physical barrier to reinnervation, and/or functions as a tissue sealant can be desirable in some embodiments.

### In Situ Forming Gels.

Of interest in some cases are in situ crosslinking synthetic polymers. In situ forming materials can be advantageous because they can be injected through a fine gauge needle as a liquid to the target zone and then form a solid scaffold in vivo that matches the contours of the potential space. In situ forming gels may transition from a solution to a gel as a result of pH, temperature, salt, light, biomolecules, solvent-exchange, UV-irradiation, ionic crosslinking, covalent crosslinking, electromagnetic field. Different types of crosslinking are described in U.S. Pub. No. 2014/0363382 A1 to Campbell et al., which is hereby incorporated by reference in its entirety.

Cross-linked. For cross-linked gels, in which two precursor solutions are typically mixed containing functional groups that react with each other to form a crosslinked gel, by varying the ratio of the precursor solutions, the concentration of an accelerator or crosslinking agent, the rate at which the two solutions form a solid hydrogel can be varied. Upon mixing the two precursors (low viscosity solutions approximating that of water), but before the formation of the solidified hydrogel, an ‘intermediate’ state of the gel in which the viscosity is between that of the precursor solution and the solidified hydrogel forms and can be injected into the TPGS and travel to the desired target level, up to 12 levels away, preferably 4-5 levels away, more preferably 2-3 levels or 4 to 15″ inches away in some cases.

In another embodiment, one of the precursor solutions (A) is delivered first to fill the target levels followed by the second precursor solution (B) which crosslinks with precursor A from the distal to proximal target sites. In yet another embodiment, saline is delivered first to clear the TPGS and aid in the creation of the channel prior to administration of the first and second precursor solutions (A/B). In another embodiment, the precursor solution A is delivered first followed by the 50/50% mix of the two precursor solutions (A/B). In one embodiment, saline is only injected in a small bolus to confirm location of the needle tip or catheter in the right location but does not predilate this space.

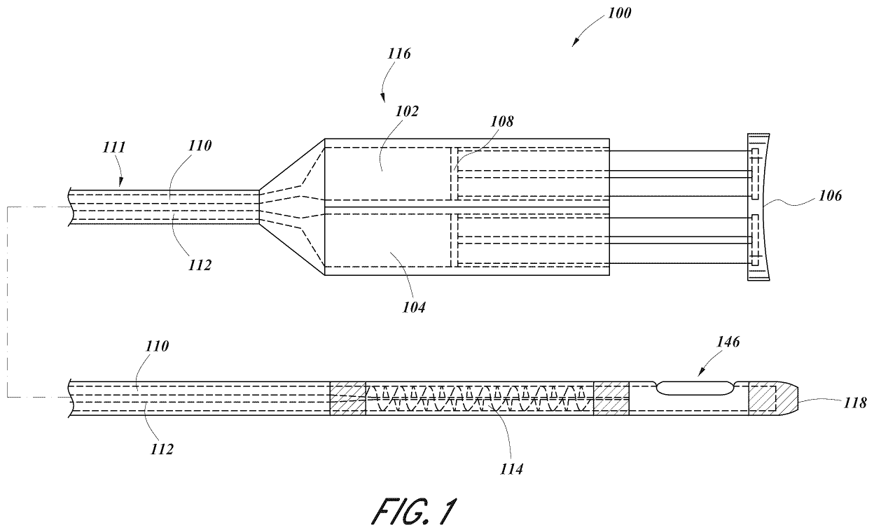

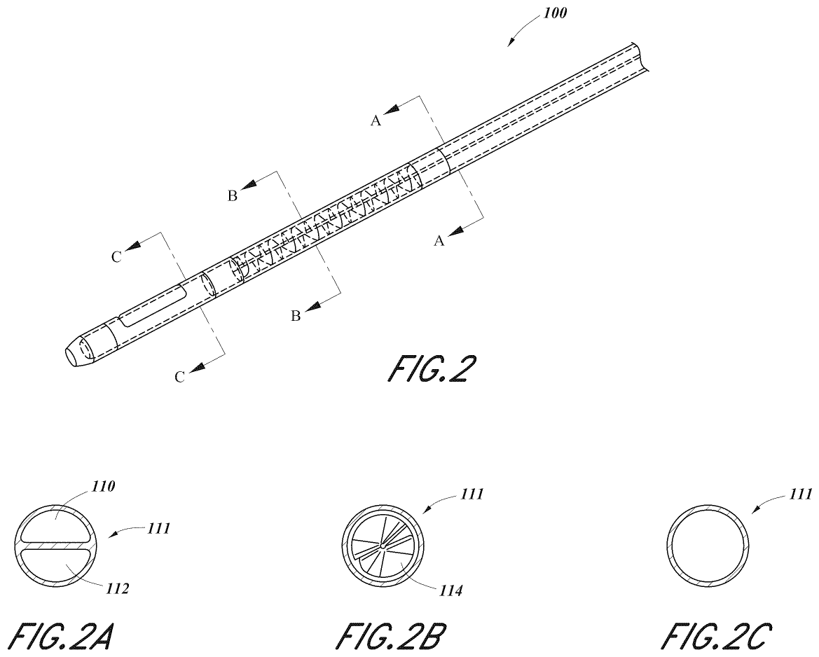

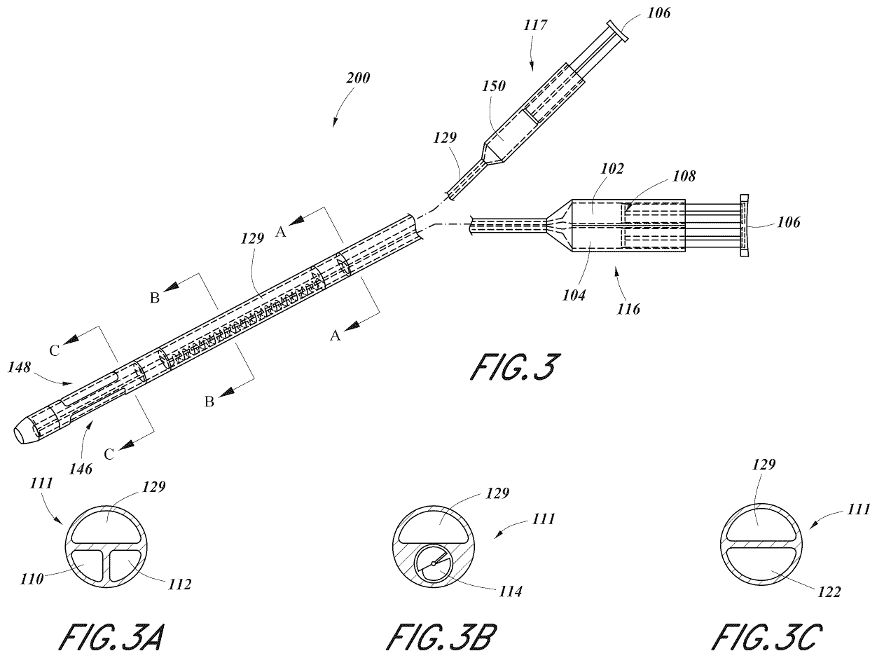

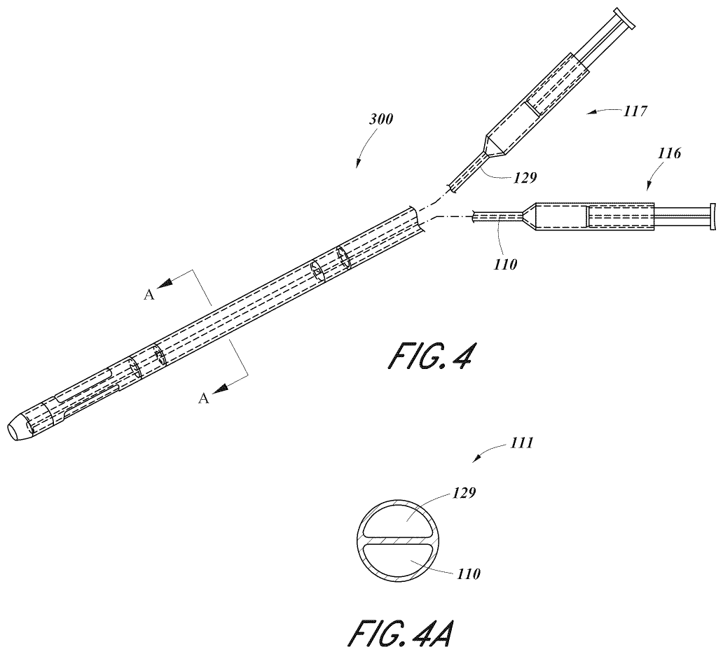

FIG. 1 illustrates an embodiment of a therapeutic agent delivery system including a catheter 100 that can interface, e.g., removably interface with a syringe or other therapeutic agent housing 116 that can include a first chamber 102 configured to house a first precursor solution 102A and a second chamber 104 configured to house a second precursor solution 104B. The housing 116 can also include a control 106 such as a plunger at its proximal end. The distal end 108 of the plunger 106 when actuated can move the solutions 102A, 104B distally through an input port (e.g., a luer port) of the catheter 100 and downstream into discrete first and second lumens 110, 112 within an elongate shaft 111 of the catheter 100. The lumens 110, 112 can in turn merge into static mixer region 114 to create a mixed solution, which can be a cross-linked gel in some embodiments as described elsewhere herein. In other embodiments, the chambers 102, 104 can be directly proximate a single lumen which facilitates mixing within the lumen. The gel can be delivered to a target location via a distally or side-facing exit port 146 at the distal end 118 of the catheter 100. In some embodiments, a curved or bent needle (not shown) can be configured to extend radially outwardly from exit port 146 to assist with targeting depending on the desired clinical result. In some embodiments, the curved or bent needle can allow the catheter to be positioned endovascularly in a blood vessel proximate the target tissue (e.g., the paravertebral gutter), and the curved or bent needle can extend through the wall of the blood vessel into the paravertebral gutter for injection of the therapeutic agent(s). In some embodiments, the delivery catheter 100 can be delivered over a guidewire (not shown), and the delivery catheter 100 can have a proximal guidewire input port, a discrete guidewire lumen, and a guidewire exit port on the distal end, such as a distally-facing exit port. FIG. 2 illustrates a distal portion of the catheter 100. FIG. 2A is a cross-section through line A-A of FIG. 2 (illustrating the elongate shaft 111 and lumens 110, 112); FIG. 2B is a cross-section through line B-B of FIG. 2 (illustrating the elongate shaft 111 and mixer region 114); FIG. 2C is a cross-section through line C-C of FIG. 2.

FIG. 3 is a schematic illustration of a delivery catheter system including one or more therapeutic agent housings (e.g., syringes) removably connected to a catheter 200 configured to deliver a plurality of therapeutic agents into different anatomical locations, according to some embodiments of the invention. The catheter 200 can include a first input port that interfaces, e.g., removably with a first syringe or other therapeutic agent housing 116 that can include a plurality of chambers housing precursor solutions for, for example, a cross-linked gel as described in connection with FIG. 1 above. Actuation of a plunger 106 or other control on the proximal end of the first housing 116 will cause the plurality of precursor solutions to flow into first and second lumens 110, 112 within the first input port and through the elongate shaft 111 of the catheter 200, and distally the first and second lumens 110, 112 are in fluid communication with a distal static mixer region 114 as previously described. Distal to the mixer region 114 the mixed solution (e.g., a cross-linked gel) flows distally into a common lumen 122, and out a distally or side-facing first exit port 146. The system can also include a second therapeutic agent housing 117 that can include only a single chamber 150 as shown (or a plurality of chambers in other embodiments). In some embodiments, the chamber 150 can be configured to house a blank protective hydrogel or other therapeutic agent as described herein. Actuation of a plunger or other control 106 will move the therapeutic agent distally, such as through a second input port which can be a luer or other connector, and through a third lumen 129 extending distally through the elongate shaft 111 of the catheter 200, and distally past (but separated from and not merging into) the mixer region 114, and out a distal or side-facing second exit port 148 spaced apart, such as spaced radially apart from the first exit port 146. This advantageously allows for, in some embodiments, a first hydrogel (e.g., including a neurolytic agent) can be delivered in a first direction (e.g., caudally), while a second hydrogel (e.g., including a protective agent) can be delivered in a second direction different from (and in some embodiments opposite) the first direction (e.g., rostrally). In some embodiments, this can allow for sympathetic neuromodulation (e.g., denervation) of the thoracic sympathetic ganglia within the paravertebral gutter while protecting the inferior cervical sympathetic ganglia within the paravertebral gutter when the catheter is positioned proximate T1/R1. In some embodiments, a plurality of curved or bent needles (not shown) that can be jointly or independently actuated can be configured to extend radially outwardly from exit ports 146, 148 in different directions to assist with targeting depending on the desired clinical result.

In some embodiments, the delivery catheter 200 can be delivered over a guidewire (not shown), and the delivery catheter 200 can have a proximal guidewire input port, a discrete guidewire lumen, and a guidewire exit port on the distal end, such as a distally-facing exit port. FIG. 3A is a relatively more proximal cross-section of the elongate shaft 111 of the catheter 200 through line A-A of FIG. 3 (illustrating the elongate shaft 111 and first lumen 110, second lumen 112, and third lumen 129); FIG. 3B is a cross-section more distally, through line B-B of FIG. 3 (illustrating the elongate shaft 111, mixer region 114 where first lumen 110 and second lumen 112 have merged, and discrete third lumen 129); FIG. 3C is an even more distal cross-section through line C-C of FIG. 3 (showing elongate shaft 111 and two lumens therein: lumen 122 (after junction of the first lumen 110 and the second lumen 112) and third lumen 129.

FIG. 4 is a schematic illustration of a delivery catheter system similar to that illustrated in FIG. 3, except the first therapeutic agent housing 116 has only a single chamber fluidly connectable via a first input port on the catheter 300 to a first lumen 110. The second therapeutic agent housing 117 also can have a single chamber fluidably connectable via a second input port on the catheter 300 to a second lumen 129. The first therapeutic agent housing 116 can house a “preformed” hydrogel (e.g., including a neurolytic agent) that does not necessarily require precursor solutions or mixing immediately prior to infusion. The second therapeutic agent housing can include a blank hydrogel or protective agent (e.g., hyaluronic acid) as previously described. FIG. 4A is a cross-sectional view through line A-A of FIG. 4.

Crosslinked PEG. In one embodiment, a hydrogel such as one from the group of in situ polymerizing poly(ethylene glycol)-based hydrogels is selected for the delivery of drugs. Crosslinked PEG-based polymers are biocompatible, have controlled crosslinking, degradation, flexibility, and relatively high adhesion strength. In particular the use of multi-arm PEGs, such as 4-armed PEG that are functionalized to cross-link with one another can be of interest. Additional spacers can be added between the 4-armed PEGs to vary the mechanical and drug delivery properties (if desired) of the polymer. The molecular weights of the PEG arms, on average, may be between about 200 Da to 20 kDa, preferably between about 1 kDa and 8 kDa, more preferably between about 2 kDa and 5 KDa in some embodiments. The molecular weight of the PEG precursor can be, in some embodiments, between about 4 KDa and 100 kDa, more preferably between about 8 kDa and 10 kDa or 20 kDa and 35 kDa. Generally, about 4 to 30% w/w concentration of precursors are used to prepare gels in some embodiments. In some embodiments, the PEG can be cross-linked with human serum albumin.

The precursors may be a combination of an ester group on one PEG (precursor A) and a trilysine amine (precursor B). In some embodiments, the precursor A is a 20 kDa N-hydroxysuccinimide end capped PEG which is resuspended at the time of delivery in sodium phosphate buffer, the accelerator. The precursor B can be, in some cases, a trilysine acetate in a 0.075 M sodium borate decahydrate buffer (pH 10.2). A preservative may be added, for example butylated hydroxytoluene (BHT). In another embodiment, the PEG precursor is a higher molecular weight 31.5 kDa N-hydroxysuccinimide end capped PEG, with the same buffer and trilysine acetate buffer, which together form a gel in about 10 seconds. In this embodiment, the PEG precursor (lyophilized) is mixed with a diluent (e.g., the trilysine acetate buffer) in a dedicated syringe. The accelerator, the sodium phosphate buffer remains in a separate syringe.

These hydrogels remains in the paravertebral gutter for, e.g., between 2 to 3 months and then erode through hydrolysis, are resorbed, and fully cleared through renal filtration within, e.g., approximately 4 to 6 months. These in situ polymerizing hydrogels have been commercially developed as an absorbable perirectal spacer (SpaceOAR), and as a dural sealant (DuraSeal, Covidien). In addition to these technologies, other types of major hemostats, sealants and adhesives described by Mehdizadeh and Yang, Macromol. Biosci. (March 2013) are incorporated by reference in its entirety. By varying the ratio of the precursors, the in situ gelation time can be varied. Newer PEG hydrogel formulations have less swelling, which can be an advantageous characteristic in a formulation delivered adjacent to the spine.

Non-limiting examples of specific hydrogels that may be employed include absorbable surgical sealants including but not limited to SpaceOAR® (Augmentix), DURASEAL™ (Medtronic), COSEAL™ (Baxter), FOCALSEAL (Genzyme), CORGEL® Biohydrogel, EVICEL® or OMNEX® (ETHICON), PROGEL™ Pleural Air Leak Sealant™ (BARD Davol Inc), the BioGlue® Sealant/Adhesive (Cryolife) and HYASIS® or Provisc™ (Novozymes) and TISSEEL/HEMASEEL (Baxter). For example, hydrogels described in U.S. Pat. Nos. 6,656,200, 5,874,500, 5,543,441, 5,514,379, 5,410,016, 5,162,430, 5,324,775, 5,752,974, and 5,550,187, incorporated by reference in their entireties, can also be utilized with systems and methods as disclosed herein.

Of these, not to be limited by theory, SpaceOAR, Progel, Tisseel, and BioGlue can in some cases have more favorable characteristics for the paravertebral gutter given their longer duration of gel time, limited swelling, longer time until degradation, improved durability in situ, and more limited gel expansion, and improved strength compared to the other tissue sealants. Fibrin sealants may also be desirable due to their limited swelling but can suffer from poor mechanical properties and more rapid clearance.

Other hyaluronic acid/hyaluronan hydrogels can also be utilized with systems and methods as disclosed herein. For example, the Corgel Biohydrogel may be utilized for drug delivery. The hydrogel includes tyramine substituted sodium hyaluronate (NaHy) with di-hydroxyphenyl linkages which are crosslinked using peroxidase (horseradish peroxidase, HRP, 10 U/ml) and hydrogen peroxide in PBS (1.0%). By varying the tyramine substitution percentage between 1 and 10% and tyramine substituted hyarluonan (TS-NaHy) concentration, the gelation properties vary.

Injection of between 0.5 and 5 ml of a microparticle slurry to the target nerve may be desirable. The slurry may be comprised of drug-loaded hydrogel macroparticles or be a blank hydrogel. For example, injection of TraceIT® (Augmentix) to the nerve can allows for unequivocal visibility under CT or MR. Biodegradable fiducial markers such as those described in US 2011/0142936 A1, hereby incorporated by reference, may be particularly suitable for the delivery of neuromodulatory drugs. The microparticle slurry may confer the conformal filling desired with minimal swelling (<70%, preferably <20%) using a 10 kDa 4-arm PEG and between 0.1 and 1% covalently attached iodine with 3-4 terminal triodoo benzoate (TIB).

Gelation time. In some embodiments, in situ crosslinking polymeric formulations can have a gelation time or sol to gel transition between 1 second and 30 seconds, preferably 2 seconds and 20 seconds, preferably between 3 seconds and 5 seconds.

### Examples of Characteristics of a Hydrogel

Equilibrium swelling. For applications in which hydrogels are delivered to nerves to prevent nerve regeneration, maintaining close adherence and apposition between the nerve and the conformable hydrogel is desirable. As a result, minimizing the equilibrium swelling post-hydrogel delivery is desirable. The equilibrium swelling occurs during in the minutes to days as the hydrogel equilibrates with the fluids in the in situ environment. It is preferable to keep the equilibrium swelling at less than 30%, more preferably less than 20% and even more preferably less than 10%. Furthermore, in some embodiments, it is desirable to avoid hydrogels that shrink as these hydrogels may compress the nerve and result in aberrant nerve firing and therefore it is preferable to use hydrogels that swell greater than 0%.

Equilibrium swelling may be assessed in vitro at room temperature, body temperature or elevated temperature conditions. At high temperature, the gel swells less than at low temperature. Swelling at 37° C. in PBS is relevant to the conditions in vivo. It is preferable to have the gel swelling between 0% and 30%. Hydrogel samples were prepared in cylindrical silicone tubing (6 mm) and cut to dimensions of 6 mm diameter by 12 mm length. Samples were weighed and merged into PBS at 37° C. After swelling in PBS for 12 hours at 37° C., samples were taken out and weighted again. The swelling is calculated by the percentage of mass increase.

### Impacts of Degradation on Mechanical Integrity of the Hydrogel Mass.

In some instances, if the degradation rate is too rapid, the hydrogel may fracture and fall off the nerve or be cleared before the hydrogel can serve the function to prevent nerve outgrowth and/or neuroma prevention. In other instances, there may be a substantial loss of the mechanical integrity within the hydrogel as a result of degradation that the nerve may extend out into the softened or fractured hydrogel and result in neuroma formation. As a result, it is preferable that a biodegradable system have no more than 50% of the hydrogel bonds cleaved at 3 months, more preferably 30%, and even more preferably 20%. Thereafter the degradation can continue until the hydrogel is entirely cleared. The loss of bonds can be evaluated in part through the reduction in the mechanical integrity of the hydrogel. Thus, it is desirable that the hydrogel maintain a compression modulus of about 40 kPa at 3 months post-administration.

### Flexibility.

Due to the forces experienced in vivo, it is desirable to have flexible robust hydrogels that will not fracture in the highly mobile and compressive environment of the body. As a result, more flexible hydrogels are desired. The flexible and robust gel can be made with longer arm length and higher crosslinking density, such as the 4-arm 20K PEG ester, 4-arm 15K PEG ester, 4-arm 10K ester combined with trilysine, or 4-arm 10K, 4-arm 15K PEG ester/amine combined with the 8-arm 20K or 8 arm 10 k PEG amine/ester.

Nondegradable hydrogels. If a nondegradable hydrogel system is used, the same equilibrium characteristics apply but, since the hydrogel is nondegradable or biostable, fracture due to loss of mechanical strength is not a concern. Nondegradable hydrogels or hydrogels that degrade very slowly include hydrogels utilizing certain urethane, urea, or amide bonds. These hydrogels may not degrade for more than 2 years or more in vivo.

In one embodiment, a 4 arm PEG amine (—NH2) and a 4 arm PEG NHS ester are mixed in the presence of HCl. The molecular weights and ratios of the two PEGs can be varied to control the properties of the polymer. In one embodiment, after the precursors are mixed, the sol to gel transition can be quick (2-13 seconds) or prolonged (1-2 minutes), to allow the gel time to migrate within the paravertebral gutter before removing the delivery system. In some embodiments, the liquid forms a gel in about 2 seconds, 10 seconds, 20 seconds, 120 seconds, or 240 seconds. In another embodiment, the crosslinked PEG hydrogels, described above, are injected without a neurolytic agent. In another embodiment, a neurolytic agent is loaded into the precursor A phase. In another embodiment, a neurolytic agent is loaded into the precursor B phase. In yet another embodiment, there is one neurolytic agent loaded in the precursor A phase and another drug loaded in the precursor B phase.

In another embodiment, hyaluronic acid is added to the precursor formulation to increase the viscosity of the solution in order that it can travel up and down the paravertebral space to the cover the target thoracic levels, and then gelling after that. For example, the PEG/HA mixture can be delivered between the T2 and T3 ribs (or between R2 and R3) and the agent flows out of the needle/catheter both rostrally and caudally. The ultrasound probe is advanced rostrally with the flow of the agent and when it reaches the lower border of the 1^{st} rib, the flow of material is halted. In another embodiment, when the materials reach the middle of the border of the 1st rib, the flow of material is halted. In some cases, when the material reaches the superior or most rostral border of the first rib, the flow is halted and the caudal spread of the agent is noted prior to removal of the needle. In one embodiment, HA is crosslinked with bifunctionalized maleimide-PEG-maleimide polymer using enzymatic crosslinking and then crosslinked with a DA click chemistry reaction to have outstanding shape memory and anti-fatigue properties.

In yet another embodiment, the crosslinked PEGs can be mixed with low molecular weight PEG, such as PEGs with a molecular weight less than 3.35 kDa, including 200 Da, 400 Da, 1 kDa, or 2 kDa, 3.35 kDa, 5 kDa linear end-capped PEGs. These PEGs can assist in modulating the release of drugs from the polymer. The low molecular hydrogels may also improve the sealing and stabilization of the damaged neuronal membranes that result from delivering the RF therapy or the neurolytic agent.

These crosslinked PEGs can be delivered through needles, such as for example 17G or 18 G needles or with needles as high as 33G, or about 27G, giving them flexibility in terms of routes of administration (catheter-based or needle-based).

Other technologies that may be adapted for use with systems and methods as disclosed herein include the Focal Seal product, which forms in situ through photochemical/chemical polymerization of acrylate-capped PEG-PLL and poly(trimethylene carbonate), or CoSeal, is a covalently crosslinked PEG product comprised of two 4-arm PEGs with glutaryl-succinimidyl ester and thiol terminal groups.

PEG Generally. PEG-based hydrogels are biocompatible, have controlled degradation, flexibility, and relatively high adhesion strength, particularly when crosslinked. Through careful selection of the molecular weight, the number of arms, and the reaction conditions, other in situ forming PEG hydrogels can be synthesized. The drug delivery systems may be comprised of functionalized linear PEG or multi-arm PEG derivatives (with reactive groups) such as those available from JenKem Technology or Nanocs. These functionalized systems may be crosslinked with one another through a covalent interaction. PEG may be functionalized with an amine group (or other acid reactive chemical group) that binds to a carboxylic group (or other amine reactive group). These include 3 arm PEG amine (—NH2), 4 arm PEG amine (—NH2), 4 arm PEG carboxyl (—COOH), 4 arm PEG SCM (4 arm PEG NHS ester), 4 arm PEG Succinimidyl glutaramide (-SGA) with a longer half-life than the -SCM) 4 arm PEG Nitrophenyl carbonate (-NPC) with a carbonate linker between the PEG and NHS ester in which the release of p-nitrophenol can be traced by UV spectroscopy, 4 arm PEG succinimidyl carbonate (-SC) with a carbonate linker and a longer half-life than-SCM, 4 arm PEG Maleimide (-MAL) which is selective for thiol groups and reacts at pH 5-6.5, 4 arm PEG Acrylate (-ACLT) for use in vinyl polymerization or co-polymerization, 4 arm PEG Thiol (—SH), 4 arm PEG Vinylsulfone (-VS) which binds to free thiol groups in aqueous buffer between 6.5 and 8.5 pH at room temperature, 4 arm PEG Succinimidyl Succinate (-SS) with a cleavable ester linker to make it a biodegradable hydrogel, 4 arm PEG Succinimidyl Glutarate (-SG) with a ester linker, 4 arm PEG Isocianate, 4 arm PEG Azide, 4 arm PEG norbornene. Similar reactive groups described above can be used with other multi-arm PEGs such as 2-arm, 3-arm, 4-arm, 5-arm, 6-arm. 7-arm, 8-arm, 9-arm, 10-arm, and more PEGSs. The molecular weight of these polymers may vary from 1 KDa to 500 KDa. In a preferred embodiment, the polymer includes 4 arms although PEG-arms may increase to 16 arms. Similarly, any of the aforementioned polymers can be combined to form co-polymers, e.g. PEG-co-alginate, PEG-co-hyaluronic acid, etc. Alternatively, heterobifunctional PEGs, methoxy PEGs (-acrylate, -aldehyde, -amine, -biotin, -carbonate, -carboxyl, -hydrazide, -maleimide, -NHS, -oligopeptide, -phospholipid) can be used, and the like. In addition to these, Lipid-PEG derivates are also available.

### Thermosensitive

In another embodiment, the gel may be an in situ thermosetting/thermosensitive gel, which requires a change in temperature to form a physical gel, typically at or below body temperature but it can be administered through a single lumen or channel without a need for mixing. The concentration of polymer can be such that it is in a low viscosity state at room temperature (for example, 23-25° C.) and a higher viscosity state at body temperature, or just below body temperature at 35° C.

Biodegradable PEG-based copolymers have been fabricated to degrade through hydrolytic, enzyme-catalyzed or mixed mechanisms. The majority of these ABA triblock, BAB and AB diblock copolymers are thermosensitive polymers that gel below body temperature, although some transition from in the opposite direction (gel at and above body temperature). These are not covalent bonds but the gel is formed through ionic or nonionic interactions, such as through chain alignment between their hydrophobic-hydrophobic regions. By controlling the molecular weight of these blocks, the gel transition temperature can occur between, e.g., 25-37° C., more preferably 30-35° C., more preferably 30-33° C. The % w/v of these gels is typically between 5 and 50% concentration, preferably between 5 and 40% concentration, more preferably between 10 and 20% concentration. Examples of amphiphilic ABA/BAB triblock and AB diblock copolymers follow: The hydrophilic A segment in this case is the PEG or PEO and the hydrophobic B segment is most a PPs/polyester/POE/PHB or a PEO penetrating the inner cavity of cyclodextrins. PEG di-block and tri-block copolymers can be formed with polyesters including PEG-PLA, PEG-PGA, PEG-PCL, MPEG-PCL, PEG-PLGA, PEG-LA-PEG, PLGA-PEG-PLGA, PEG-PLGA-PEG, PEG-PCL-PEG, PEG-PGA-PEG, PCL-PEG-PCL or with trimethylene carbonate (PEG-TMC), PEG-chitosan, PEG-dextrose, PEG-gelatin, and other suitable combinations of polymers may be selected. In another embodiment poly(ethylene oxide-co-glycidol)-CHO is formed by mixing aqueous glycol chitosan and poly(EO-co-Gly)-CHO to form a cross-linked hydrogels in situ. Alternatively, an α-cyclodextrin/PEG-b-PCL-dodecanedioic acid-PCL-PEG hydrogel (MPEG-PCL-MPEG) showed promise for cardiac applications delivering cells and may be suitable for use in the paravertebral gutter. Alternatively, a four-arm PPO-PEO block copolymer (Tetonic) can be modified with acrylates for crosslinking and NHS-group added for reaction with tissue amines. PMID 20298770. Alternatively, the PEO-CMC hydrogel (Oxiplex, MediShield, Dynavisc, Aril, FzioMed) has many of the characteristics to make it an excellent polymer to deliver drugs to the paravertebral gutter. Still other polymers include, PEO-PHB-PEO hydrogels. PEG-PCL-PEG or PCL-PEG-PCL (PCEP) which transition from a solution at room temperature to a gel at body temperature are described. For example, in one embodiment, a PEG-PCL-PEG hydrogel (2K-2K-2K) forms a thermosensitive hydrogel that can be injected as a solution and forms a gel in situ. Neuroprotective drugs can be safety mixed into the hydrogel solution prior to injection in situ. Also, pH-block copolymer hydrogels may be well suited for this application and may include diblock copolymers such as PEG-PCL, PEG-PLA or triblock copolymers such as PEG-PLGA-PEG.

Pre-formed PEG hydrogels. In another embodiment, PEG can be crosslinked ex vivo, dehydrated and then crushed. These particles can then be resuspended in an aqueous buffer with or without drug and stored in a preloaded syringe for injection. The advantage for this type of delivery system is the ability to provide clinician with the drug delivery system ready for use. One example of this technology is the TraceIT hydrogel (Augmentix), which is an injectable hydrogel that is visible under ultrasound, CT, and MR that can be injected with a 25G needle and remains in place for approximately three months and gradually degrades through hydrolysis and is bioresorbed over 7 months. The iodinated PEG confers the visibility under CT and MR. In one embodiment, a PEG (non-iodinated) slurry is injected into the paravertebral gutter with a wt % of between 2.5% and 20%. The neuromodulatory agents described may be incorporated into the hydrogel. Drugs with low solubility may be incorporated as crystals, particulates, or in a suspension. Higher water solubility drugs, incorporated in a hydrogel, typically only release for hours to days. If they are additionally incorporated into microspheres, liposomes, or nanoparticles, their release rate can be delayed and they can provide more sustained release. Further examples can be found, for example, in U.S. Pub. No. 2014/0363382 to Campbell et al., which is hereby incorporated by reference in its entirety.

Hyaluronic acid. The hyaluronic acid (HA) can be formulated with a range of viscosities and modulus of elasticities. Since it is shear-thinning or thixotropic, it can easily be injected through higher gauge needles and after it is injected the gel returns to its intramolecular and intramolecular ionic links are restored. As the shear force is increased, such as during injection, the hydrogel becomes thinner (shear-thinning) allowing the delivery of some hydrogels through a standard syringe needle or catheter such as a 27 G or 29 G thin walled needle or a 30 G needle, as necessary.

By varying the molecular weight of HA, the degree of crosslinking and the concentration of reactive HA precursors, hydrogels of varying pore size and viscosity and degradation rate can be produced. HA is negatively charged and so it can absorb a lot of water and expand forming a loose hydrated network. The HA may be in the form of randomly crosslinked HA chains and neuromodulatory agents can be encapsulated in the network without any covalent linkage. HA can be reacted with an excess of glycidyl methacrylate (GMA) to form crosslinked HAHA can be crosslinked with bisepoxide, divinyl sulfone derivatives under alkaline conditions, glutaraldehyde, biscarbodiimide and hydrazides under acidic conditions.

HA-based hydrogel particles (HGPs) also known as microgels or nanogels can be synthesized from water in oil emulsion crosslinking to form aqueous droplets of HA. These microscopic gels provide a convenient method to deliver drugs in the aqueous phase inside these gels.

Considerable work has gone into developing HA-based gels to solve the various needs of dermal fillers based on if tissue plumping or filling versus small wrinkle filling are needed. As a result, these gels have a wide variety of viscosity after injection. The complex viscosity (n*) relates to how the hydrogel flows from the needle and then later how much it spreads. Generally, Restylane SubQ>Perlane>Restylane, in that order, are more viscous hyaluronic acid fillers than Juvederm, Voluma>Juvederm Ultra Plus>Juvederm Ultra which have low viscosity. In these embodiments, it is preferably to have a hyaluronic acid based delivery system with a higher viscosity filler so that the agent will remain in place.

The following hyaluronic acid/hyaluronan based products include, for example, Perlane, Juvederm (Ultra, Ultra XC, Volume XC), Restylane and Hyalform, and collagen-based products such as Evolence. Perlane is more viscous than Restylane containing particles between 750 and 1000 microns, similarly Juvederm's line contains hyaluronic acids with different viscosities/thicknesses.

Another advantage to hyaluronic acid based products beyond their extensive clinical evaluation is that it is possible to dissolve excess filler with hyaluronidase. In one embodiment, the glycosidic bonds of hyaluronic acid can be cleaved with Vitrase (ovine hyaluronic acid, 200 USP/ml) which can be injected by itself or with saline into the site containing the hyaluronic acid to assist in the diffusion of fluid and clearance of the hyaluronic acid. For example, in one embodiment 20 mg/ml of crosslinked hyaluronic acid (cross-linked with BDDE) is suspended in PBS at neutral pH. Lidocaine (0.3%) can also be incorporated the gels to reduce the pain associated with injection Hyaluronidase is also delivered locally to increase nerve permeability and is sometimes used in conjunction with 10% hypertonic saline as a neurolytic agent and to break up adhesions in the spine (1500 U/10 ml). Conventional hyaluronic acid hydrogel crosslinking can be employed, as disclosed, for example, in U.S. Pat. No. 4,582,865 to Balazs et al., which is hereby incorporated by reference in its entirety. In some embodiments, the viscosity of an HA gel is greater than 0.65 kg/m³, or between about 1.56 kg/m³ to 2.80 kg/m³ depending on molecular weight.

Ethanol based systems. With hydrophobic drugs and hydrogel monomers or hydrogels are soluble in ethanol, a high drug-loaded hydrogel can be created. Since ethanol can act as either a solvent for the polymer as well as a neurolytic agent and the alcohol is rapidly absorbed once placed in the body, novel hydrogels using alcohol may be possible. In one embodiment the neurolytic agent is coadministered with the hydrogel in an aqueous/ethanol solution. The ethanol, between, for example, 10 and 50 wt %, more preferably 30%, can be incorporated in a HA- or PEG-based hydrogel. With regard to the in situ forming crosslinked hydrogels, the ethanol can either be incorporated in the precursor solution prior to mixing the agents and formation of the gel. This may be reflected in the kit in which the alcohol is an additional vial. In one embodiment, between about 1% to 75% ethanol, preferably between about 20% to 75% ethanol is delivered in the gel formulation. For example, ethanol may be loaded into a hyaluronic acid gel comprising 25% ethanol, 75% saline, and hyaluronic acid (molecular weight average 1 kDa or molecular weight average 2 kDa). In some embodiments, alcohol can leave, and/or drugs can precipitate out of the hydrogel.

In another embodiment, the active agent is added to the polymer solution where it is either dissolved (soluble) or dispersed (insoluble-suspension/dispersion) in the polymer solution. After the solution is injected into the target site, the solvent (ethanol) diffuses away from the polymer-drug mixture while water diffuses in, causing the polymer to turn into a solid drug delivery implant. The drug is subsequently released by diffusion or dissolution. In one embodiment the drug is dissolved in ethanol and the monomers PEG methyl ether (MPEG)-PLA, acrylol chloride macromonomer, itraconic acid, and MPEG methacrylate to form poly(LA-IA-MEG). In one embodiment, ethanol is added to the aqueous phase of the polymer and modifies the gelation time. Addition of ethanol, for example 25% ethanol, improves the mechanical properties of the gel.

In some embodiments, the viscosity of the injectate is comparable to water. In these embodiments, the complete infiltration of, for example, a neuroma or painful post-surgical scar tissue is desired. In these clinical indications, the injectate can spread to fill the space and then form a crosslinked hydrogel in situ. In some embodiments, the viscosity of the injectate is greater or less than the viscosity of the final formulation, such as at least about, about, or no more than about 10%, 20%, 30%, 40%, 50%, 60%, 70%, 80%, 90%, 100%, or more or less greater, or ranges including any two of the foregoing values.

### Poloxamers.

The Pluronic class of polymers are nonionic triblock copolymers of poly(ethylene oxide)-poly(propylene oxide)-poly(ethylene oxide) (PEO-PPO-PEO) that are thermoreversible polymers that are thought to form as micelles aggregate together above the critical micellular concentration (CMC) to form a gel. Poloxamers form hydrogels as homopolymers or as uncomplexed multi-block copolymers. Poloxamer properties can further be controlled through crosslinking to improve the release of drug and modify the sol-gel transition behavior and critical gelation temperature and concentration. Poloxamers, such as P407, can be injected into the potential space and used to protect tissues encapsulated in the semi-solid gel from thermal damage such as RF, ultrasound, and radiation. Poloxamers form at between 10 and 60% wt/volume, more preferably between 20 and 50%, more preferably 25-35% wt/vol. The P407 is thermoreversible (15.4% in water) and transitions to a semi-solid at body temperature. Pluronic F-127 is a nonionic surfactant polyol (MW 12.5 KDa) with 7% PPO that at low concentrations forms micelles and at high concentrations packs to form high modulus gels. HPMC can be added to Poloxamers to prolong the gelation time. In another example, a polaxamer-heparin hydrogel if formed from poloxamer (PEG-propylene glycol-PEG). In another example, 20% ethanol is added to the Poloxamer solution without affecting the concentration for gelation. At 30% ethanol and 35 wt % F-127 can form at 20 degrees Celsius. As another example, two Pluronic block copolymers can be mixed to vary the properties of the gel. In one embodiment, Pluronic F127 can be loaded with the neurolytic agent and then F-127 can be mixed with F-68 to assist in reducing the gelation temperature.

Other polymers. The aforementioned not limiting, there is an unmet need for an injectable gel, that includes a glue, slurry, scaffold, or hydrogel- or a more simple emulsion or other viscous solution formulation that can deliver a neuromodulatory agent or combination of neuromodulatory agents. In some embodiments, the therapy can include neuromodulatory agent(s) delivered in a gel. In some embodiments the neuromodulatory agent is co-delivered with an anesthetic and/or contrast agent. In some embodiments, the anesthetic, if delivered, is administered immediately prior to the injection of the therapy.

Formulations include gels, and more particularly hydrogels that can form either through physical crosslinking (ionic interactions, hydrogen bonding, hydrophobic-hydrophobic interactions) or chemical crosslinking (Schiff base crosslinking, Diels-Alder crosslinking, Michael addition, CuAAC, SPAAC, Thiol-ene, Oxime, and Radical polymerization. The polymerization of hydrogels can be induced by physical mixing, temperature, pH, UV light exposure, and/or ionic concentration. Polymeric gels may be homopolymers, copolymers, or multi-polymer interpenetrating polymeric hydrogels. The gels may be nonionic (neutral), anionic, cationic, amphoteric electrolytes (ampholytic, acid and base groups), or zwitterionic (anionic and cationic groups in each structural repeating unit).

Echogenicity In some embodiments, the gel can be sufficiently echogenic to allow the clinician administering the therapy to confirm its appropriate delivery within the paravertebral space as well as to track its subsequent spread up and down the paravertebral gutter. In some embodiments, the gel has low to no internal pores, decreasing the rate water permeation through the gel, decreasing the rate of drug release, and preventing the ingrowth of neuronal and non-neuronal cells.

After the gel has formed at the site or has been delivered to the site, the gel may provide for sustained or controlled release of the agent. This can provide more effective means to deliver therapeutic or neurotoxic concentrations locally to the target tissue. In the case of a neurolytic agent, this can allow more complete denervation of the nerves that are in direct contact with the gel whether through 1) encapsulated or surrounded with the gel, 2) partially surrounded by the gel on one side and another anatomical structure on another side (blood vessel, bone, organ, adipose, fascia, extracellular matrix, lymph node etc.), or indirectly via drug diffusion across extravascular tissues or adipose tissues. By completely filling the potential space, the thread-like rami communicants that are not visible to the naked eye are also destroyed. This can be advantageous since if they are not transected or cauterized during a surgical sympathectomy or RF percutaneous sympathectomy procedure, they provide a surviving pathway or, alternatively, a pathway for appropriate regeneration or reinnervation of fibers later.

Blank gel. In another embodiment, blank (non-drug loaded hydrogel) can be injected to off-target neural structures to act as a buffer to prevent drug spread to neural or other tissue that needs to be protected from the effect of the neuromodulatory agent. Subsequently, a drug-loaded hydrogel can be delivered to the desired levels for ablation. The blank hydrogel can be injected up against with the other neuromodulatory-gel. In the case of short-acting agents, the blank gel only needs to protect adjacent neural tissue as long as the neurolytic agent is released and so the gel in some cases preferably degrades faster in the tissue than the neurolytic-loaded hydrogel. In a further embodiment, the blank and neurolytic-loaded.

Degradable bonds on the PEG hydrogel. PEG hydrogels with a hydrolytically degrdable ester bond after crosslinking can be made with PEG-succinimidyl succinate (SS), PEG-succinimidyl glutarate (SG), PEG-SAZ, PEG-SAP can provide several months of mechanical integrity prior to degradation. Modifying them to include an aromatic ester, urea, or urethane bond may extend the degradation profile of the hydrogel. Other active bonds include amide, carbamate, carbonate, urea, thiourea, thioester, disulfide, hydrazone, oxime, imine, amidine, triazole and thiol/maleimide. The degradable bonds can be on the polymer itself or form upon the gelation with polymer or small molecules with active groups such as active ester, amine, aldehyde, ketone, isocyanate, thio-isocyanate, thiol, maleimide, azide, alkyne, imidate, hydrazine. Of particular interest is the development of hydrogels with longer persistence in vivo than can be achieved with the commonly used PEG-esters. Additional active bonds that have more extended degradation in vivo such as the PEG-ureas (e.g. PEG isocyanate, PEG-NCO), PEG-urethanes (PEG-succinimidyl carbonate) (PEG-SC) and PEG-carbamate are of interest. Hydrogels comprised of polyethylene glycol succinimidyl carbonates (PEG-SCs) with more than 2 arms, such as the 4-arm, 6-arm, or 8-arm PEGs with molecular weights ranging from 1K to 50K, preferably 10K to 20K, such as 10K, 15K or 20 kDa. In some embodiments, the 4-arm 10K PEG-SC, 4-arm 20K PEG-SC, 8-arm 10K PEG-SC, 8-arm 15K PEG-SC, or 8-arm 20K PEG-SC are selected, more preferably 4-arm 10K PEG-SC or 8-arm 20K PEG-SC. The following patents are incorporated for reference: application No. 20160331738A1.

Polyurethanes. Incorporation of soft segments in the to the polyurethane may also be desirable. In some embodiments, the soft segments include poly(caprolactone), poly(lactic acid), polyhydroxyalkanoates (PHA), poly(ethylene adipate) (PEA), alipathic diisocyanates such as isophorone diisocyanate (IPDI) or L-lysine ethyl ester diisocyanate (LDI). Amino acids can also be incorporated as chain extenders in the PEG-SC. These soft segments improve the degradability of the polyurethanes. In some embodiments low molecular weight trifunctional polyester polyols are selected for incorporation. Please refer to FIG. 1—common monomers used for synthesis of biostable and biodegradable polyurethanes, incorporated herein for reference (Chapter: Degradation of Polyurethanes for Cardiovascular Applications, Book: Advances in Biomaterials Science and Biomedical Applications).

Blends. In some embodiments, it may be desirable to create blends of two PEGs to improve the degradability of the system. In one embodiment, the PEG-SC is combined with PEG-SG prior to crosslinking with trilysine amine to create a hydrogel that has sufficient mechanical support to prevent nerve outgrowth but then degrades more rapidly than PEG-SC. The persistent time of gel in vivo is fine-tuned by the ratio of PEG-SG and PEG-SC. With the increase of PEG-SC content, the persistent time of gel in vivo increases. In another embodiment, the PEG-carbamates are blended with the PEG-carbonates.

Hydrogel made with nanoparticles, micelles and microparticles. In some embodiment drug formulas can be either dissolved or suspended in the diluent or accelerator. Surfactant can be added to stabilize the suspension. The drug can be also encapsulated in microparticles nanoparticle or micelles and then suspended in diluent or accelerator. In some embodiments, the hydrogel can be made with cross-linkable particles and micelles. These particles or micelles have reactive groups such as the active ester, amine, carboxyl, thiol and those described in patent U.S. Pat. No. 7,347,850 B2 and can be crosslinked with small molecules, polymers, particles or micelles with reactive groups which reacts with the former particles or micelles and forming bonds including amide, carbamate, carbonate, urea, thiourea, thioester, disulfide, hydrazone, oxime, imine, amidine and triazole. In other embodiments, gel can be form by the swelling of particles. The large volume of swelling can increase the particle contact and lock them into their location to form gel.

Solid content. The solid content can be adjusted to fine tune the swelling and tensile properties of the hydrogel. For example, the solid content can be adjusted above the critical gelation concentration, such as between 6 to 15% loading, more preferably 7-9% loading, more preferably 8-8.5% solid content.

Crosslinking. Hydrogels may be formed in situ through electrophilic-nucleophilic, free radical, or photo-polymerization.

### Tensile and Compressive Strength.

The tensile strength of the hydrogels can be critical to the performance of the hydrogel in vivo. In particular, maintaining the hydrogel in situ without mechanical collapse or fragmentation that would result in a disruption to drug release profile and/or a disruption in the ability to provide a physical barrier to nerve regeneration. Refer to a range of tensile moduli disclosed before. Generally, it is preferable to select hydrogels with tensile properties greater than 40 kPa.

| Polymer | Tensile Modulus | |
|---|---|---|
| Formulation A | 10-15 | kPa |
| Cyanoacrylate | 60-70 | kPa |
| Formulation C | 70 | kPa |
| Formulation D | 200 | kPa |
| Formulation E | 50 | kPa |
| Formulation F | 20 | kPa |

Procedure. Tensile properties of hydrogel formulations were measured at a 1 mm/min strain rate using a universal load frame (Instron Model 5943). Hydrogel samples were prepared in cylindrical silicone tubing (6 mm) and cut to dimensions of 6 mm diameter by 100 mm length. Samples were allowed to swell in PBS for 12 hours at 37° C. before mechanical testing. The modulus was calculated as the tangent slope of the linear region (between 0.05 and 0.17 strain level) of the stress-strain curve.

Compressive strength.

In some embodiments, samples are harvested from the subcutaneous space in rats, cut to 100 mm long, pre-equilibrated (as per above) and evaluated for compressive strength.

| Polymer | Compressive modulus | |
|---|---|---|
| Formulation G | 0-10 | kPa |
| Formulation H | 20-35 | kPa |
| Formulation I | 70 | kPa |
| Formulation I - After 3 months in vivo | 50 | kPa |
| Formulation J | 20 | kPa |
| Formulation J - After 3 months in vivo | 10 | kPa |

Procedure. Compressive properties of the hydrogel formulations were measured at a 1 mm/min with the Instron. Hydrogel samples were prepared and equilibrated as per above. The modulus was calculated as the tangent slope of the linear region between 0.05 and 0.17 of the stress-strain curve.

In vivo Mechanical Properties. In some embodiments, it is desirable to have persistence of mechanical properties for over 2 months, more preferably 3 months or more to maintain the integrity of the hydrogel around the nerve.

In vivo Persistence. In some embodiments a longer in vivo persistence may be preferred, in which the hydrogel remains in situ for between 3 months and 3 years, more preferably 6 months and 18 months, more preferably 9 months and 13 months.

### Adhesion.

Adhesive strength is an important criterion for maintaining the hydrogel in close apposition to the nerve. Adhesion may occur through crosslinking reactions between the hydrogel and the primary and secondary amines on the tissue surface e.g. the epineurium. The adhesion strength should be greater than 10 kPa, preferably greater than 50 kPa, more preferably greater than 100 kPa.

Adhesive strength on nerves can be estimated by embedding the sciatic nerve in the hydrogels. The ends of the nerves are embedded in superglue between sandpaper and placed in titanium clamps in a Bose ElectroForce 3200-ES. Nerves are pulsed at a rate of 0.08 mm/s until failure. Care was taken to ensure that the nerves were used shortly after harvest and that the hydrogel and nerve were equilibriated in PBS at 37° C. prior to testing.

Example 1. Adhesion forces are evaluated on a 4-arm PEG-SC hydrogel crosslinked with trilysine amine. Forces were in the range of 90 to 200 kPa.

Other hydrogels. In situ forming polyanhydrides are also of interest for developing applications directed towards nerves. In one embodiment, polyanyhdydrige polymers can be acrylated so that they can form in situ through free radical polymerization. Alternatively, they can form through photocrosslinking. At lower concentrations, the polymers are water soluble e.g. 10%. The prevention of nerve regeneration is conferred in part through their hydrophobicity. Incorporated by reference in their entireties are US20180177913A1 and US201562181270.

In some embodiments, it may be desirable to combine a non-growth permissive hydrogel (e.g. crosslinked PEG hydrogel, alginate, methacryloyl-substituted tropoelastin MeTro hydrogel) with a growth permissive hydrogel (e.g. gelatin-methacryloyl GelM, GelM/PEG or GelMA/MeTro composites) PMID: 29580168. The growth permissive hydrogels permit the migration of Schwann cells within the hydrogel. In another embodiment, a crosslinked chitosan hydrogel may provide sufficient mechanical properties to permit the nerves to regenerate.

### Amiodarone Loaded Hydrogels

In some embodiments, hydrogels are loaded with amiodarone with or without the addition of ethanol. For example, 0.1 to 5 wt % loading of amiodarone or more can be achieved. This can also be accomplished and improved with the incorporation of ethanol into the solution. For example, 50 to 75% ethanol can be incorporated with 0.25 wt % amiodarone to achieve burst release of amiodarone between 3 to 5 days. Similarly, 1% amiodarone can be delivered from the hydrogels for a period of 30-60 days.

### Example 1

In some embodiments the 4-arm-PEG 10K-SC is crosslinked with 8-arm PEG 20K amine. The PEG-SC and PEG-amine were dissolved in an acidic diluent at a ratio of 1:1. The suspension was mixed with accelerator buffer and formed hydrogel through a static mixer. This formulation gelled in 4 seconds.

### Example 2

In other example, 8-arm 15K PEG-SC is crosslinked with trilysine. The PEG-SC were suspended in buffered trilysine solution and then mixed with accelerator buffer through static mixer. This formulation gelled in 2 seconds and the gel provided compression strength up to 200 kPa.

### Example 3

In other example, 8-arm 20 K PEG-thioisocyanate is crosslinked with trilysine at a ratio of 1:1. The formulation gelled in 3 seconds and has a compression strength of 120 kPa and 5% swelling.

The in vivo degradation, swelling, compressive and tensile strength, gelation time of the hydrogel all play a critical role in determining the appropriate hydrogel for delivery to nerves.

Equilibrium swelling. For applications in which hydrogels are delivered to nerves to prevent nerve regeneration, maintaining close adherence and apposition between the nerve and the conformable hydrogel is desirable. As a result, minimizing the equilibrium swelling post-hydrogel delivery is desirable. The equilibrium swelling occurs during in the minutes to days as the hydrogel equilibrates with the fluids in the in situ environment. It is preferable to keep the equilibrium swelling at less than 30%, more preferably less than 20% and even more preferably less than 10%. Furthermore, in some embodiments, it is desirable to avoid hydrogels that shrink as these hydrogels may compress the nerve and result in aberrant nerve firing and therefore it is preferable to use hydrogels that swell greater than 0%.

Equilibrium swelling may be assessed in vitro at room temperature, body temperature or elevated temperature conditions. At high temperature, the gel swells less than at low temperature. Swelling at 37° C. in PBS is relevant to the conditions in vivo. It is preferable to have the gel swelling between 0% and 30%. Hydrogel samples were prepared in cylindrical silicone tubing (6 mm) and cut to dimensions of 6 mm diameter by 12 mm length. Samples were weighed and merged into PBS at 37° C. After swelling in PBS for 12 hours at 37° C., samples were taken out and weighted again. The swelling is calculated by the percentage of mass increase.

### Impacts of Degradation on Mechanical Integrity of the Hydrogel Mass.

In some instances, if the degradation rate is too rapid, the hydrogel may fracture and fall off the nerve or be cleared before the hydrogel can serve the function to prevent nerve outgrowth and/or neuroma prevention. In other instances, there may be a substantial loss of the mechanical integrity within the hydrogel as a result of degradation that the nerve may extend out into the softened or fractured hydrogel and result in neuroma formation. As a result, it is preferable that a biodegradable system have no more than 50% of the hydrogel bonds cleaved at 3 months, more preferably 30%, and even more preferably 20%. Thereafter the degradation can continue until the hydrogel is entirely cleared. The loss of bonds can be evaluated in part through the reduction in the mechanical integrity of the hydrogel. Thus, it is desirable that the hydrogel maintain a compression modulus of about 40 kPa at 3 months post-administration.

### Flexibility.

Due to the forces experienced in vivo, it is desirable to have flexible robust hydrogels that will not fracture in the highly mobile and compressive environment of the body. As a result, more flexible hydrogels are desired such as combinations of the 4-arm 10K or 20 K PEGs with 4-arm or 8-arm 20K PEG-amines may be desirable.

Nondegradable hydrogels. If a nondegradable hydrogel system is used, the same equilibrium characteristics apply but, since the hydrogel is nondegradable or biostable, fracture due to loss of mechanical strength is not a concern. Nondegradable hydrogels or hydrogels that degrade very slowly include hydrogels utilizing certain urethane, urea, or amide bonds. These hydrogels may not degrade for more than 2 years or more in vivo. For example, in some instances, 4-arm PEG succinimidyl glutaramide 10K (SGA) may used in combination with 8-arm PEG-amine 20K at 8% solid content.

Polymers. The drug delivery system may be comprised of a nondegradable polymer such as silicone, cellulose or ethylene vinyl acetate copolymer (EVAc), polystyrene, acrylamide, or cyanoacrylate glues. However, in some embodiments, the drug delivery system is comprised of biodegradable or bioerodible polymers. The drug delivery systems may be comprised of natural polymers including, but not limited to glycosaminoglycans and polysaccharides including but not limited to collagen, alginate, chitosan, pullulan, hyaluronic acid, hyaluronan, gelatin, carboxymethylcellulose (CMC) silk fibroin, dermatan sulfate, chitin, and chondroitin sulfate and derivatives thereof. Synthetic biodegradable polymers such as polylactic acid (D-, L-, D/L, PLA), polyglycolic acid (PGA), polylactic-co-glycolic acid (PLGA), polyaminoacids, polyorthoesters (POE), polycaprolactone (PCL), polyphosphoesters (PPE), poly(urethanes), polyanhydrides, polyimide, propylene glycol, poly(ethylene oxide), olyethylene glycol (PEG), poly(2-hydroxyethyl methacrylate) (PHEMA), and poly N-(2-hydroxypropyl)-methacrylamide (PHPMA), poly(methylmethacrylate) (PMMA) (Artecoll or Artefill-microspheres in a collagen gel), polyacrylamide (Aquamid) poly(ester urethane), cyclodextrin, poly(alkene oxide), poly(hydroxyalkanoate), poly(R-3-hydroxybutyrate) (PHB) and co-hetero-polymers thereof. Other components include glycerol, poly(glycerol-co-sebacic acid), and poly(ethylene oxide) (PEO) These polymers can be further modified to create hydrogels with cholesterol methacrylate or 2-ethoxyethyl methacrylate (EOEMA). The polymers can include linear backbones or star or branched polymers with molecular weights ranging from 1 kDa to 500 kDa, more preferably 2 kDa to 300 kDa. Some examples include but are not limited to poly(epsilon-caprolactone-co-ethyl ethylene phosphate, a copolymer of caprolactone and ethyl ethylene phosphate (PCLEEP), polilactofate-PLA (PPE-PLA) copolymer (Paclimer Microspheres), polyanhydride-co-imide, poly(TMA-Tyr-:SA:CPP 20:50:30) polymer (Chiba et al), poly(vinyl alcohol) based cryogels. For these purposes, polyscaccharides, N-isopropylacrylamide (NIPAAm) copolymers (thermosensi), poloxamer and its copolymers, pEO-P(D,L)LGA copolymers and liposome based systems. In one embodiment, copolymerization of NIPAAm, acrylic acid and hydroxymethacrylate and TMC (HEMAPTMC) may be suitable for injection.

Additional biodegradable polymers, solvents, aqueous carriers, are described in, for example, U.S. Pat. No. 6,545,067 to Buchner et al. and U.S. Pub. No. 2014/0363498 to Sawhney et al., both of which are incorporated by reference in their entireties).

Natural gels based gels: Chitosan-β-glycerophosphate/hydroxyl-ethyl cellulose (chitosan/β-GP/HEC) hydrogels, chitosan-polylysine hydrogels, alginate hydrogels, and collagen hydrogels can also be utilized in some embodiments, as can rapid gelling hydrogels composed of mixtures of chitosan-thiol modified and polylysine-maleimide give gelation times of between, e.g., about 15 and 215 seconds. These hydrogels have excellent hemostatic properties. In another embodiment gelatin methacrylate can be utilized.

Fibrin-based gels. Chondroitin sulfate proteoglycan gel (CSPGs), such as Aggregan, Neurocan, Brevican, Versican, and NG2 exert inhibitor influences on axon growth as can urinary bladder matrix (UBM). Fibrin and fibrinogen, whether mammalian or non-mammalian, may be used as an injectable gel but may be less desirable because of its ability to support neurite extension. Matrigel and other fibrin gels in some cases do not stay around for long enough to prevent regeneration. However, fibrin may be conjugated with PEG to improve its characteristics. In one embodiment, the drug is delivered in a crosslinked fibrin matrix, sealant glue or slurry, such as the FDA approved Tisseel. By varying the concentration of thrombin used to induce polymerization, the solution to gel transition can be controlled.

Other commercial formulations that may be suitable include collagen based gels such as Evolence (with Glymatrix technology), calcium hydroxyapatite microspheres (CaHA, Radiesse), and pro-fibrotic PLLA microspheres (Sculptra), and/or the fibrin matrix or glue (Tisseel) made of fibrin and thrombin.

Biodegradable alginate or collagen, or agarose-chitosan hydrogels. In one example a chitosan hydrogel is prepared by mixing chitosan (2% w/v) with dibasic sodium phosphate (DSP) to for a gel that at body temperature. In one embodiment, the BST-Gel platform (Biosyntech, Canada) is utilized, that includes chitosan neutralized with beta-glycerophosphate (GP) which forms a gel at room temperature.

Pullulan. In another embodiment hydrogels made from pullulan, a natural polysaccharide, that have excellent oxygen barrier properties, highly transparent, and is non-hydroscopic, may be used. Acutely, the polymer may result in pH changes in situ that may be toxic to neurons. In one embodiment pullulan is modified to form pullulan methacrylate (PulMA) to hydrogels to deliver drugs.

Thermochemical ablation. The use of liquid alkali may be a safe and effective method to ablate nerves. Permeable oil-packed alkali metal sodium-potassium (Na-K), in which the oil controls the rate of heat release during the Na-K reaction with water in living tissue. Alternatively, the delivery of a single electrophilic reagent, such as acetyl chloride (AcCl, 4 mol/L) or acetic anhydride (Ac(2)O) can be delivered in vivo to cause a significant pH change or temperature increase of around 300C (PMID 23311380). Two component systems containing HCl or acetic acid in either NH₄OH or NaOH also have potential. Similarly, the exothermic reaction caused by the initiators and the hydrogel polymerization in situ can liberate heat and cause nerve degeneration.

Delivery of cells for applications where neuroregeneration, neuronal survival, or neuroprotection are desired. In another embodiment, pluripotent cells can be delivered in the hydrogels to differentiate into neurons, glia, or other supporting cells within the sympathetic chain. These transplanted cells can provide for the release of growth factors, cytokines and anti-inhibitory molecules to promote regrowth, to target the sympathetic afferent and efferent nerves. In one embodiment, these cells secrete nerve growth factor (NGF).

Retrograde. In some embodiments, the drugs can be delivered to the ganglia after injection into the pericardial sac or into the heart. In these embodiments, the drug can be delivered transcutaneously into the pericardial sac or other target heart location. The drugs are taken up at the nerve synapse and are retrogradely transported back to the sympathetic efferent ganglia located in the sympathetic chain or the afferent visceral nerves located in the dorsal root ganglion. In another embodiment, drugs are delivered locally within the pericardial sac to target the interneurons located in the ganglionated plexi located around the heart.

Anterograde. In other embodiments, it can be desirable to deliver the drug from the sympathetic chain to the afferent and efferent nerve terminals/synapses in the heart. In this manner, drugs that will be taken up at the soma or dendrites and delivered after anterograde transport to the heart and/or lungs.

Circumferential. One of the challenges with injecting neurolytic agent around a vessel is achieving circumferential delivery of the drug. In one embodiment, a pre-configured fiber is injected transvascularly out of a curved needle and the polymer self-forms a coil-like shape around the vessel that provides sustained release of a neurolytic agent circumferentially around the vessel. The noodle may have a curved shape within the lumen of the needle or it may assume a curved shape as it exits the lumen of the needle and comes in contact with water.

Mechanism of drug release. In applications requiring the sustained release of a neurolytic agent for days to weeks but the prolonged presence of a drug delivery system such as a hydrogel to prevent nerve regeneration, the release of the drug is in some embodiments not controlled by the degradation of the polymer. Sustained release gels may additionally incorporate complexes, microspheres, nanospheres, nanocrystals, micelles, liposomes, nanoliposomes, or nanocomplexes, as known in the art. Alternatively, a viscous formulation such as a suspension, emulsion or a slurry can be delivered to the tissue, such as a slurry of hydrogel particles, in which the release rate is primarily controlled by the environment into which it is injected. Drug diffusion through gels can also be controlled by the polymer concentration, the degree of swelling (hydration factor).

Microspheres. In order to provide more controlled release and reduce the burst, the drugs may be loaded into microspheres. These microspheres can be delivered in a slurry or incorporated into a hydrogel. In one embodiment, the microspheres are incorporated into an in situ forming hydrogel. In another embodiment they are incorporated into a lyophilized phase of the in situ polymerizing hydrogel in which they will only get resuspended when they are ready for use. The microspheres may release the neuromodulatory agent with or without neuromodulatory agent also loaded in the hydrogel phase. Alternatively, the microspheres may release one agent and the aqueous phase of the hydrogel may release a different agent. In this embodiment, the release rates of the drug from the microsphere and gel phase may differ. Typically the release of drug from the microspheres will be slower than that from the hydrogel. In some embodiments, the microspheres are biodegradable so that they are eventually cleared from the site of injection.

Microspheres can be formed by single or double-emulsion. In one embodiment, a poly(ethylene glycol) based microsphere system if formed with a water-in-water emulsion process. A single (W/O) or double W/O/W emulsion process can be used to prepare the drug. By adjusting the number of sites of hydrolysis, emulsion conditions and varying the PEG molecular weight the degradation and erosion can be controlled. In one embodiment, PEG-diacrylate (PEGDA) chains are reacted with dithiol molecules to form hydrolytically labile ester linkages proximal to thioether bonds, PEG-dithiol (PEG-DTT). A water-in-water emulsion process is then used to synthesize the PEG microspheres. Alternatively, the PEG-DTT polymer solution can be dispersed in a 40 kDa dextran-rich aqueous phase and the acrylate groups in the droplets can be crosslinked with UV light to form microspheres. The microspheres are removed from the emulsion by dilution of the dextran-rich phase and centrifugation.

Nanoparticles If intracellular delivery of these agents is desired, the neuromodulatory agent can be encapsulated within nanoparticles which are more readily endocytosed into the cells. Alternatively, the gold nanoparticles can be conjugated directly to the neuromodulatory agents as these readily accumulate within neurons.

Nanocrystals. For example, a drug may be formulated in nanocrystals and dispersed in a drug delivery system. The crystals can be sieved to achieve a particular range of particle size in order to better control the release of drug. Alternatively, the drug may be micronized to reduce the size of the drug particles.

In some embodiments, the drug release occurs through diffusion of the drug from the drug delivery system. In one embodiment, the drug crystals are loaded into the hydrogel, and the release of the drug occurs as the hydrogel absorbs water after implantation causing solubilization of the hydrophobic drug crystal and subsequent sustained diffusion into the surrounding tissues, thus the polymer hydrogel itself is imparting

Coprecipitates. Instead of microspheres, the poorly water soluble drugs may be complexed with one or more pharmacological carriers. In one embodiment an inert water-soluble carbohydrate is selected to form a coprecipitate with a neuromodulatory agent in order to better control the release profile of the drug. For example, the drug can be coprecipitated with fructose, polydextrose or xylose at a ratio of drug: carrier of between 1:5 to 1:20.

Embedded drug delivery systems to facilitate controlled release of drugs from the hydrogels include The drug is loaded into microspheres in a hydrogel that provide the rate-limiting release of the drug. The polymers may degrade by bulk or surface erosion over a period of days to weeks to months, as needed for a given application. For example, in one embodiment, a thermoresponsive Poloxamer gel is combined with pH sensitive chitosan nanocomplexes containing the active agent.

Polymer conjugation. The polymer may be conjugated to the drug with an enzymatic or hydrolytic linkage. In one embodiment, the linkage is a hydrolytic linkage off of the backbone of is the polymer and upon delivery into an aqueous environment, hydrolysis causes release of the drug.

Lipophilic for depots. Highly lipophilic agents may be particularly desirable agents to deliver to nerves and are efficient in forming depots in the fascia and adipose tissue through which these nerves run.

Differential sensitivity. In another embodiment, a chemical agent is delivered that is preferentially more sensitive to one type of neural fiber than another. For example, sympathetic efferent fibers are recognized to be more sensitive to anesthetic than sensory afferent fibers. In another embodiment, the soma themselves are targeted such as the sympathetic ganglia or the dorsal root ganglia.

A further embodiment includes adding proteolytically degradable sites in the PEG system, enabling both proteolytic and hydrolytic or mixed-mode degradation.

Free base. Alternatively, the drug can be converted to its free base, where applicable, and injected or delivered as a viscous paste directly or incorporated within a drug delivery system.

Drug loading levels. The drug loading level can be in some embodiments about 1% to 80%, about 5 to 50%, or about 5 to 20% in some cases

Volumes of agent or formulation administered. Although the physician will have the discretion to deliver the appropriate volume of therapy to the paravertebral gutter, the following table provides a guide to volumes injected according to some embodiments. More typically volumes from about 1 ml to 30 ml are delivered in and around various neural targets. In the paravertebral gutter, volumes between about 1 ml and 20 ml are delivered to treat the target vessels or organs, more typically between about 2.5 and 10 ml, or 1 and 5 ml.

Gel set times. In situ crosslinkable agents can be formed in which both reagents, typically with at least one of them a polymer, are modified with a functional group to allow crosslinking. By varying the concentration of the agents, the degree of substitution of the active/functional groups, and the molar ratio of the two crosslinking agents, the gelation can be modulated. In gels that have a sol-gel transition, this can occur from between 1 second to 5 minutes, in some cases between 1 to 3 minutes. This may refer to the time after initiation (e.g. temperature change, pH change, crosslinker mixing) until the formulation is no longer injectable or flowable, even if it hasn't yet reached it maximal strength characteristics.

Minimal or non-swelling. In some embodiments, the formulation should not result in a clinically significant change in size, such that upon delivery there is less than 50%, preferably less than 30% or more preferably less than 10% swelling. The swelling may be cationic or anionic or, in the case of cross-linked hydrogels it may be a function of cross-link density.

Durability of effect. Agents may be delivered that cause short-term denervation followed by axonal sprouting and regeneration. Agents may be delivered that result in long-term permanent denervation either by destroying fibers that are not capable of regeneration, by destroying a long enough region of the axon or axon bundle that the fibers cannot regenerate or lack the appropriate trophic factor support to guide the fiber regeneration back to the original source, or by destroying the soma themselves. Agents may be delivered that provide for an inhibitory environment to prevent axon regeneration.

In some embodiments, a gel including one or more therapeutic agents can be delivered to or proximate the celiac plexus. The procedure can be performed under fluoroscopy in which lidocaine/bupivacaine is first injected through the RF needle, ablative energy delivered, and then the in situ forming hydrogel is administered. Another example is delivery to or proximat the splanchnic nerve for the treatment of chronic pain, such as that from a pancreatic malignancy. The procedure can be performed unilaterally or bilaterally.

For applications involving delivery of neuromodulatory agents around nerves and limiting off-target neural and non-neural spread, a flowable injectate can be desirable. The formulation can be flowable but viscous enough to retain in the target injection region. The advantages of higher viscosity agents are not only that the injectate can help to retain the agent at a therapeutic concentration at the injection site, sustain the release of the agent from the formulation.

The viscous injectate may include a swollen network or gel including an organogel an aerogel or a hydrogel, a viscous liquids, foams, a slurry, a paste, a semi-solid, a spray that can be delivered through a 17 gauge needle or higher. The gel may comprise of a synthetic liquid polymer.

In some embodiments, the viscosity of the injectate is sufficient to retain the formulation at the site. These formulations do not require a crosslinking or polymerization agent in order further increase the viscosity or change the mechanical characteristics of the formulation. In some preferred embodiments, the formulation is injected in situ at one viscosity and then changes viscosity upon injection. This may be a result of a transition in temperature from room temperature to body temperature, from an acidic or basic to a neutral pH, from the diffusion of an excipient out of the formulation, from the addition of a crosslinking agent that crosslinks the backbones of polymers, a crosslinking agent that crosslinks the side chains of a polymer, such as a star-shaped polymer with multi-functional agents. By modifying the number of functional groups on the polymers, the molecular weight of the polymer, the molecular weight of the crosslinking agent, the concentration of the crosslinking agent, the ratio of the polymer to the crosslinking agent, it is possible to develop a formulation with the characteristics sufficient to retain the mechanical integrity of the formulation in vivo.

The hydrogels may be physically or chemically crosslinked in vitro or in situ. The crosslinked hydrogels may be hydrolytically degraded, enzymatically degraded, thermally degraded.

Degradation or erosion and/or resorption. The degradation, erosion, absorption or clearance of these drug delivery systems can be in some embodiments between one week and one year, preferably one week and 6 months, more preferably between one and three weeks in the blank hydrogel embodiments, and between one week and 9 months in the neurolytic-hydrogel group, preferably between 2 to 6 months. If the system undergoes enzymatic or hydrolytic degradation, this begins after the nerves have become chronically degraded, e.g., between two or three months. The scaffold or hydrogel can be designed to fully resorb within the body after the period for axon sprouting and attempts at reinnervation/regeneration is over. Typically this will be on the order of 2 weeks to 1 year, more preferably 2 weeks to 6 months, more preferably two to four months. The hydrogel may be biodegradable or bioerodible and can be ultimately cleared from the site.

Timing of the delivery of the formulation. In one embodiment, the therapy is delivered in as a one-time inpatient or outpatient procedure. However, repeat procedures may be necessary if there is some reoccurrence of symptoms. In the preferred embodiment the hydrogels degrade within two to three months after injection and the re-establishment of connections is prevented. Thereafter the hydrogel may be gone should an additional procedure be required.

Frequency of administration. Current chemical neurolysis approaches may not be as long lasting as energy-based approaches such as radio- or cryofrequency for achieving nerve degeneration. As a result, chemical denervation approaches such as phenol or alcohol may in some embodiments require a second or third application/procedure in order to maximize effectiveness. Effectiveness may be maximized by either extending the lesion, treating more fibers, or preventing regeneration. In one paper 19/23 patients required one injection of alcohol in the lumbar chain only and the remaining 4 required a second block.

In another embodiment, the neurolytic agent is administered first and then one or two weeks later, the biodegradable hydrogel is inserted.

Example 1. Sustained release. HA was conjugated to 4-arm PEG-amine (10 kDa) with 1-ethyl-3-(3-dimethlaminopropyl)-carbodiimide hydrochloride as a cross linker at a 100:1 ratio. Reserpine crystals were then incorporated into the HA/PEG hydrogel at a loading level of 150 μg in 10 ml hydrogel. When injected into the subcutaneous space, the reserpine was released at 15% within the first 1 hour, 50% within 6 hours, 80% at 18 hours and 100% at 24 hours.

Example 2. In another embodiment, NHS-ester activated chondroitin sulfate is crosslinked with 6-arm PEG amine. After an initial burst of 15% of the drug loading, the reserpine is mixed in to release approximately 20-50 μg per day from the hydrogel for 3 to 4 weeks.

Example 3. In one embodiment, NGF is delivered from an in situ forming thermosensitive hydrogel, such as PEG-PCL-PEG or heparin-poloxamer (HP) gel such as those used for delivering NGF to the spinal cord to treat spinal cord injury in rats (PMID 26472614). Additional hydrophilic and hydrophobic polymeric additives, such as PVA, PEG or PCL can be added to vary gel concentration or drug release. In another embodiment, NGF is released from a crosslinked 20 kDa 4-arm PEG homopolymer. The larger size of the molecular delivered permits the sustained release of NGF without any additional additives.

Example 4. In another embodiment, NGF is delivered in a diblock copolypeptide hydrogel (DCH) to serve as a depot for drug release at a drug delivery rate in steady state at about 20 ng/ml.

Example 5. In one embodiment solutions of covalently crosslinked multi-armed PEG hydrogel particles of about 70 μm in diameter are formulated in a PEG (20 kDa) water solution to improve the injectability of the slurry.

Example 6. Valproate-loaded chitosan nanoparticles were prepared. The chitosan solution (0.3% (w/v) in 5.54% sodium acetate, pH 5.5) was added dropwise to the continuously stirring mixture of tripolyphosphate (TPP, 2.5% w/v) and sodium valproate (25 mg/ml). The slightly negatively charged chitosan nanoparticles form through ionotrophic gelation with particle sizes less than 100 nm a loading level of up to 50% and drug release for about a week. The nanoparticles are loaded in a PEG-PLGA-PEG triblock (33 wt % solution, MW 3300) hydrogel, resulting in valproate drug release for a week and persistence of the hydrogel in vivo for over a month.

Example 7. A Poly(N-isopropylacrylamide) (pNIPAm) based thermosensitive microgel was loaded with desipramine (50 mg/ml) hydrochloride, a cationic drug, which binds to the polymer via the carboxyl groups. The resultant thermoresponsive polymer microgels range in size from 500 to 800 μm in water. Drug was released from the hydrogel for between 1 and 3 days.

Nanocrystals. In one embodiment, an in situ thermo-sensitive hydrogel loaded with nanocrystals (NCs) of a hydrophobic drug such as reserpine at a drug loading of up to 5 mg/ml, more preferably 3 mg/ml. In another embodiment, the gel is loaded with paclitaxel at up to 3 mg/ml. PMID 24512789. In another embodiment, a PTX-NCs-Gel system with Pluronic F-127 uses PTX-NCs and Taxol as controls.

Overall concept of drug delivery. Any number of drugs or other therapeutic agents, including those disclosed herein, can be used with any gel disclosed herein depending on the desired clinical result. At lower doses, these agents can be utilized to modulate nerves and at higher doses these agents may ablate nerves. At higher concentrations, these agents may have nerve blocking effects, reducing or eliminating the pain associated with the procedure while also exerting ablative effects. For example, nerve ablation may refer to the chemical destruction of the ganglia or cell body of the nerve. In another example, the nerve ablation may refer only to the disruption of the axon itself leading to resportion of the ablated or transected portion of the axon while the proximal axon or proximal stump survives. In the periphery, then, the nerves are largely viable, receiving trophic and nutrient support from the distant ganglia, but are in a suspended state. By delivering a hydrogel around these ablated or suspended nerves, nerve regeneration is blocked and aberrant nerve firing is greatly reduced. Chemical ablation may be desirable over thermal ablation approaches in some cases because it may reduce or eliminates pain and unpleasant sensations during the procedure. More complete chemical ablation in conjunction with an in situ forming hydrogel may reduce the inicidence of neuritis and post-operative neuralgia relating the ablation procedure. The agents may be delivered by a percutaneous, transcutaneous, or endovascular approach or even through an endoscopic or thorascopic approach. The agents may result in chemoablation or chemolysis or chemical sympathicolysis in some cases. The delivery of the therapeutic composition or agent can be controllable with respect to the number of levels above and below the injection site and the neural targets within the paravertebral space.

Generally, chemical agents that exert a specific neuromodulatory effect on neurons can be desired depending on the desired clinical result. Classes of drugs include but are not limited to ionotropic, chronotropic, metabotropic drugs that suppress neurotransmission, anti-depressants, anti-psychotics, NMDA antagonists, opioid analgesics, anti-depressants, alpha-1 or beta2-adrenergic antagonists or alpha-2 agonists, calcium-channel blockers (CCBs), anesthetics, neurotoxins, neuroablative agents, depolarizing agents, non-depolarizing agents, hyperpolarizing agents, sympathicomimetics, sympatholytics, sympathetic antagonist, sympathotoxins, immunosympathectomy agents, auto-immune sympathectomy agents, anti-neuronal immunotoxin agents, antihypertensive agents, TRPV1 antagonists or agonists, tricyclic anti-depressants, low and high affinity Na+ blockers, imidazoline receptor agonist, ganglionic blocking agents, neurotransmitters, parasympathicomimetics, corticosteroids, and anti-neoplastic drugs. These agents may be delivered alone or in combination to exert a neuromodulatory effect directly or indirectly. These agents may result in a temporary block, a long-term-block, a temporary degenerative response without cell recovery, or a permanent degenerative effect. These methods may result in reversible or irreversible effects. These agents may also have an anti-inflammatory or neuroprotective effects. These agents may improve neuronal survival. In some embodiments, it can be preferable to co-deliver anesthetic with or without epinephrine or norepinephrine in the neuromodulatory solution in the paravertebral space to reduce complications.

Objectives/Unmet Need with Drugs. Cardiac disease, and in particular heart failure, is characterized by increased sympathetic release of norepinephrine (norE), depleted cardiac stores of NE, accompanied with a defect of norE uptake in the cardiac sympathetic nerve terminals. The defect in the uptake is in part due to a reduction in noreE transporter density in the sympathetic nerve endings and may be a major contributor to the elevated myocardial interstitial norE. Increased interstitial norepinephrine reduces myocardial adrenoreceptor density, increases myocyte apoptosis, and lowers the threshold for cardiac arrhythmias. In addition, these acute surges or norE are thought to increase the propensity for myocardial infarction in patients with coronary artery disease as the resultant blood pressure surge and vasoconstriction trigger a fissure in a coronary artery plaque, providing a thrombogenic focus together with increased

Given the high levels of norepinephrine release that occur when the sympathetic ganglia are surgically transected, thermally ablated, or denervated with an excitatory neuromodulatory agent or agents that temporarily increase the levels of norepinephrine at the synaptic cleft, the development of neuromodulatory agents that directly or indirectly block or reduce the release of norepinephrine acutely, subacutely, or chronically are desirable for certain indications, particularly cardiac indications. The therapy, including a neuromodulatory agent and a carrier, can be delivered to the paravertebral gutter, containing the sympathetic chain, intermediate ganglia, rami communicants and intercostal vessels. The carrier can be, for example, a hydrogel or other viscous formulation that can be used to deliver the neuromodulatory agent. The therapy may also be delivered to intermediate/accessory ganglia lying along the nerve roots or rami communicants or along the nerves as they course to the visceral organs and vessels. Alternatively, the therapy may be delivered specifically to the region in between or surrounding the dorsal root and the dorsal root ganglion (DRG), particularly if sympathetic fibers have been identified innervating the DRG. In particular, the selection of neuromodulatory or neurolytic agents that directly or indirectly reduce the release of norepinephrine from sympathetic nerves followed by or in concert with triggering neuronal cell death such as through necrosis, autophagy or apoptosis, “dark” compacted death or a combination of these, can be desired.

In one embodiment, it can be desirable to deliver a neurolytic agent that can be taken up locally at the sympathetic ganglia (pre- or para-vertebral, may be mixed with parasympathetic fibers) and then directly or indirectly prevent or limit the release of norepinephrine at the nerve presynaptic terminal. As local intracellular or extracellular levels of the neurolytic agent rise, the drug initially modulates the nerve activity within its therapeutic range to exert a beneficial effect on reduction in norepinephrine spillover; as the drug concentration continues to rise to toxic levels it triggers pre- and/or post-ganglionic efferent neuronal cell death. At high concentrations, neurotoxicity to afferent and pre-ganglionic neurons that course through or in the paravertebral gutter is also possible. In one embodiment, the specificity of the neuromodulatory drug for the post-ganglionic nerves results in only the sympathetic efferent fibers being targeted. In yet another embodiment, the neuromodulatory drug targets only the sympathetic afferent fibers. In another embodiment, the drug targets only the pre-ganglionic fibers, reducing the extent of denervation supersensitivity. In another embodiment, such as for applications for the treatment of angina or ventricular arrhythmia, both the afferent and efferent sympathetic fibers are targeted. In another embodiment, such as for applications for arrhythmias (atrial, ventricular), it may be desirable to denervate only the pre-ganglionic sympathetic fibers, the post-ganglionic sympathetic fibers, or both fiber types. In yet other embodiments, it may be desirable to denervate the parasympathetic, sympathetic, and/or interneuron fibers in the cardiac intrinsic ganglia and plexi or subsets of neurons thereof. In another embodiment, it may be desirable to modulate only a subset or subsets of the fibers, by a different classification, such as by size and presence or absence of myelin. Similarly, it may be desirable to modulate C fibers (nonmyelinated, pain, nonlocalizing ache), temperature, touch, postganglionic autonomic), B fibers (preganglionic autonomic), or A-delta (Pain, fast-localizing, temperature, firm touch), A-gamma (muscle spindle stretch), A-beta (light touch and pressure), and A-alpha (somatic motor and proprioception) fibers differentially.

Reducing norepinephrine release can be achieved, for example, by blocking or inhibiting the action potential of the post-ganglionic neuron, blocking the release of acetylcholine from the pre-ganglionic sympathetic neuron, blocking the acetylcholine receptors on the post-ganglionic neuron, blocking the action of acetylcholine-induced depolarization, blocking the synthesis of norepinephrine (norE), blocking the transport of norE, blocking vesicular norE release and vesicle cycling, competing with or replacing norE transport into vesicles, depletion of neurosecretory vesicle content, modulating the calcium currents. This can result in a reduction on local norepinephrine levels at the tissue level and may translate to a reduction in norepinephrine spillover from the organ.

Alternatively, the neuromodulatory agent may be delivered to the neurons within the therapeutic window and provide a sustained block of norepinephrine release or neuronal activity, resulting in a long-lasting but reversible chemical sympathectomy without denervation of the nerve. In one embodiment this can be achieved by sustained release of a presynaptic alpha-2 receptor agonists such as dexmedetomidine or the non-selective alpha blocker phentolamine. In another embodiment, this can be achieved with the sustained release of reserpine, a VMAT-2 receptor antagonist, a catecholamine depleting agent. In another embodiment this can be achieved with pentolinium, a nonexcitatory ganglionic blocking agent. In other embodiments, the co-transmitters neuropeptide Y or histamine may also be reduced. In another embodiment, a local anesthetic, such as bupivacaine, results in local anesthetic neurotoxic effects, such as at concentrations above 5%, more preferably 8% or more. At higher concentrations, these agents result in neurolysis of the sympathetic visceral efferent (and also afferent) nerves innervating the thorax. Generally, because these agents are functioning through a non-excitotoxic mechanism of cell death, longer duration exposure may necessary to achieve neuronal degeneration.

In yet another embodiment, neuromodulatory agents can be delivered at the nerve terminals or synapses in the periphery at the target tissue, such as the lung or the heart. In some embodiments, the greatest proportion of cell receptors for the drugs are found at the nerve terminal or synapses. In other embodiments, the drug can cross the cytoplasmic membrane or is taken up through endocytosis or receptor-mediated endocytosis and transported retrogradely to the cell body, in this case the sympathetic ganglia to trigger apoptosis or neuronal cell death. The following drugs may be used as neuromodulatory agents, or, at higher concentrations, they may act as neurolytic or neurotoxic agents.

Action potential blockers. Drugs that block the depolarization and effectively prevent the nerve from reaching a threshold to trigger an action potential or drugs that are hyperpolarizing or prolong the hyperpolarization of the neuronal cell membrane may be effective at preventing or reducing the release of norepinephrine at the synapse. These drugs may result in an inhibitory postsynaptic potential or IPSP.

Ion channel modulation. Drugs that block calcium channels such as dihydropyridine (DHP)-type blockers (e.g. nifedipine, felodipine, nicardipine, nimodipine, and amlodipine), and non-hydropyridine blockers (phenylalkylamines (e.g., verapamil), benzothiazepines (e.g., diltiazem), nonselective agents (e.g., mibefradil, bepridil, flunarizine, fluspirilene, and fendiline). In particular, the neuronal N-type blockers omega-conotoxin GVIA (0.9 microM) which blocks NE and histamine release from sympathetic neurons but does not alter neuronal NE uptake or storage, or ziconotide, or T-type blockers such as amiloride (500 microM) which modulate dorsal root ganglion activity, L-type blockers like ethanol, verapamil, or combination L- and T-type antagonists such as lomerizine, P-, Q- and N-type blockers such as omega-grammotoxin SIA or omega-agatoxin IVA may be employed. Ranolazine, which blocks the late inward sodium current, may be beneficial if delivered locally to the sympathetic ganglia since it blocks the neuronal sodium channel and may have a role in relatively reducing sympathetic activity relative to parasympathetic. Drugs that activate potassium channels hyperpolarize and stabilize the cell membranes, reducing calcium entry, preventing vasoconstriction may be employed. These include, for example, diazoxide, minoxidil, nicorandil and pinacidil. Na+/H+ exchange inhibitors such as benzamil, cariporide, sabiporide, amiloride or the more specific derivatives, dimethylamiloride or ethylisopropylamiloride, reduce neuronal cell excitability, and markedly attenuate norE overflow which may be desired. Drugs that block sodium channels may be selected including 1,-8-cineole. Hyperpolarization-activated cyclic nucleotide-gated channels (HCN) inhibitors, such as ivabradine, lamotrigine, gabapentin, propofol, and lidocaine may be employed. The anticonvulsant valproic acid has been demonstrated to hyperpolarize sympathetic ganglia and trigger neurotoxicity at higher concentrations.

Sigma receptor (σ1Rs) agonists. Sigma receptor agonists may rapidly inhibit or block all calcium channel subtypes found on the cell body of the sympathetic neurons (N-, L-P/Q- and R-type calcium channels) at the sympathetic ganglia or intrinsic ganglia (e.g. intracardiac), accelerate calcium channel inactivation rate, and shifted the activation toward more negative potentials. Sigma-1 receptor agonists include haloperidol, ibogaine, (+)-pentazocine, and 1,3-Di-O-tolylguanidin (DTG) as well as berberine, citalopram, dextromethorphan, dehydroepiandrosterone (DHEA) and pregnenolone, fluoxetine, igmesine, ketamine, methamphetamine, methoxetamine, noscapine, phencyclidine, novocaine, prilocaine and other opioids buprenorphine, tramadol. Buprenorphine has been reported to be used in Ganglionic Local Opioid Analgesia (GLOA) blocks at the stellate ganglion for the treatment of upper body chronic pain syndromes. Antagonists to these receptors have also been demonstrated to exert anti-nociceptive actions both centrally and peripherally by modulating pain hypersensitivity. Amiodarone is a class III antiarrhythmic agent that has beta blocker-like and calcium channel blocker-like actions, increasing the refractory period via sodium- and potassium-channel effects, and has demonstrated neurotoxicity. The first generation antipsychotics chlorpromazine and pimozide or second generation less-cytotoxic antipsychotics olanzapine and risperidone, for example, are cytocidal. As with other cytotoxic agents, their cytotoxic potential is usually after the receptors have been saturated and may be related to cholesterol-related mechanisms and changes in lipid metabolism.

Anesthetics also block voltage-gated sodium (or calcium) channels and thus block nerve activity sympathetic efferent and afferent nerves and may be employed from the amino-amides, amino-esters, or other group. These drugs have demonstrated cytotoxicity in visceral sensory neurons (sympathetic) and sympathetic efferent (pre- and post-) ganglionic neurons at higher concentrations which can be used to achieve a permanent nerve block. Anesthetics (aminoesters, aminoamides) include N-butyl tetracaine (37 mM, 1.11% tetracaine-HCl) and other tetracaines, bupivacaine, ropivacaine, ketamine, lidocaine, procaine, iontocaine, chloroprocaine, EMLA, prilocaine, benzocaine, mepivacaine, neosaxitoxin, tetrodotoxin, saxitoxin, prenylamine, Marcaine, lignocaine, levobupivacaine, benzocaine, menthol, and the like. In one embodiment, the procedure is performed under ether anesthesia because these can, in some cases, result in faster depletion of norepinephrine content than pentobarbitone anesthesia after surgical sympathectomy.

In one embodiment, the local concentration and duration of anesthetic exposure can be controlled to permit differential blockade and or differential neurotoxicity. For example, the type B fibers (e.g. sympathetic efferent) may be blocked followed by the type C fibers (e.g. sympathetic or somatic afferent). Many anesthetics have multiple inhibitory effects such as lidocaine, which is also a nicotinic acetylcholine receptor blocker. At higher concentrations necrotic and apopotic (lidocaine, amitriptyline) cell death may be triggered by other mechanisms. In one embodiment, 2% lidocaine is delivered locally. In another embodiment, 5% bupivacaine is delivered locally. In another embodiment, prilocaine is administered to inhibit nerve firing immediately and may also inhibit the NET transporter.

Drugs that induce, potentiate, or increase the persistence of hyperpolarization via the 5-HT1 receptor such as 5-hydroxytryptamine, 8-OH-DPAT and 5-Carboxamidotryptamine (5-CT), haloperidol or ketanserin may be employed. This mechanism may also result in a reduction in acetylcholine from pre-synaptic pre-ganglionic sympathetic efferent neurons. Other agents that reduce the synaptic transmission in sympathetic ganglia include lysergic acid diethylamide, methysergide, and chymotrypsin. Other agents that may be of interest are drugs than enhance the uptake of norepinephrine into the nerve.

Vesicular monoamine transport (VMAT) inhibitors, and VMAT-2 inhibitors in particular, are another class of compounds that may be used to modulate or chemodenervate. VMAT 2 inhibitors include reserpine (RES, also blocks VMAT-1), bietaserpine, ketanserin, tetrabenazine (TBZ), phenylethylamine, MDMA (Ecstacy), N-methyl-4-phenylpyridinium (MPP+), non-hydrolysable GTP-analogue guanylyllimidiodiphsphate GMP-P(NH)P and VMAT-1 inhibitor fenfluramine. These drugs belong to the class of indole alkaloids and also include ajmaline, mediodespidine, desperidine, syrosingopine and rescinnamine. In particular, reserpine depletes the granular uptake and storage of catecholamines through near irreversible binding to VMAT-2, such as norepinephrine, and 5-hydroxytryptamine (5-HT) and does not excite sympathetic efferent post-ganglionic neurons, leading to a chemical sympathectomy. Resperine may be delivered locally at doses of, for example, about 0.1 to about 10 mg, about 0.5 to about 5 mg, about 1 to about 2 mg per injection at a concentration of between about 0.1 to about 1 mg/ml, such as 0.02 to 0.5 mg/ml, or 0.03 to 0.25 mg/ml.

Dyes as Therapeutic Agents. The potential of P2X7 receptor antagonists, which block the P2X7R receptor, such as Brilliant Blue FCF (BB FCF) otherwise known as FD&C#1 (https://pubchem.ncbi.nlm.nih.gov/compound/Acid_Blue_9), to improve nerve survival and regeneration after injury has been established. Similar models using Brilliant Blue G in rat models of sciatic nerve crush (Ribeiro et al 2017) and ischemia in the myenteric plexus (Palombit et al 2019). In addition, BBG is thought to have anti-inflammatory and anti-nociceptive effects through reducing high extracellular ATP concentrations and high calcium influxes after nerve damage. Similar drugs can be used to block the P2X3, P2X4 and other P2X receptors through other mechanism including modulation of the microglia. Dyes may be incorporated into the vial containing the polymer powder, the diluent, or the accelerator at a concentration of 0.0001 to 5%, preferably 0.001 to 0.25%, more preferably 0.01 to 0.02% wt % or approximately 1 to 1000 ppm, preferably 10 to 100 ppm. On a per site anatomic basis, local doses as high as 25 mg of dye may be delivered inn a hydrogel locally. For example, the FD&C#1 dye may be delivered at 0.01% concentration in the hydrogels to reduce neuronal injury after stroke. By incorporating the dye into the hydrogel, the dye may help improve the survival of the transected axons, reduce the local inflammation while the hydrogel provides a barrier to regeneration.

Neuroprotective/neurocounteractive drugs. Neuroprotective drugs can be delivered alone locally or distally or delivered in a gel or formulation to control their spread and direct the agents to the tissue that requires protection. For example, an anesthetic can be delivered to the nerve terminals to prevent neurotoxicity that is induced by excitotoxicity, NGF or CNTF can delivered to prevent neurotoxicity from vincristine and Taxol. Other agents include potassium channel blockers including amiodarone, clofilium and semtilide to counter-act, for example, potassium channel agonists. Similarly, the calcium channel antagonist flunarizine, cinnarizine, diphenylpiperazines protects against neuronal cell death in preclinical models of axon transection or crush. Dexamethasone or alpha-lipoic acid can be delivered to attenuate the neurotoxiity of bupivacaine and lidocaine and reduce provide protection to sympathetic neurons from immune-cell mediated necrosis and apoptosis such as with agents like guanethidine. MAO inhibitors have been demonstrated to counteract the chemical sympathectomy caused by reserpine. Alternatively, a high-K+ environment (greater than or equal to 33 mM), the actions of a VMAT, such as tetrabenazine (TBZ) can be blocked with a catecholamine uptake also helps to prevent sympathetic cell death. Minocycline and deferoxamine mesilate, amifostine, glutathione, diethyldithiocarbamate, Org 2766, curcumin, or vitamin E can prevent against cisplatin toxicity. Others drugs with recognized neuroprotective effects include the L-type Ca2+ channel blocker, nimodipine (2microM) has been demonstrated to be neuroprotective in both DRG and CNS, as well as nifedipine and nilvadipine, metformin, dexamethasone, estrogen (neuroprotective or neurocytotoxic), bupropion, or the MAO-B inhibitor deprenyl, or the adenosine A2A antagonist MSX-3, or topiramate (TPM) and lacosamide which stabilize hyperexcitable membranes, calcineurin inhibitors (CNI) include cyclosporin, tacrolimus, and sirolimus. As such, in some embodiments, a first agonist therapeutic agent can be delivered to a first location, and a second antagonist therapeutic agent to the first therapeutic agent can be delivered to a second location.

Nondepolarizing Ganglionic blockers. Nicotinic receptor blockers competitively block the action of acetylcholine on nicotinic receptors or block the ion channel that is gated by the nicotinic receptor. In one embodiment, nicotinic receptor blockers can be delivered locally to the sympathetic nervous system, such as the ganglia themselves, and block efferent neurotransmission irrespective of the neurotransmitter released at the nerve endings (e.g. norepinephrine, acetylcholine, histamine, NPY). Ganglionic blockers include chlorisondamine, tetraethylammonium (TEA), methyldopa, neostigmine, pempidine hydrogen tartrate, hexamethonium, decamethonium, mecamylamine, methyllcaconitine trimethaphan camsylate, trimetaphan camphor sulfonate, rocuronium, ibogaine, 18-methoxycoronaridine, dextromethorphan and pentolinium tartrate, and other polyalkylpiperidines and their derivatives. Other agents include monoxidine, amantadine, erysodine, tubocurarine chloride, varenicline, atracurium besylate, dehydronorketamine, ketamine, alpha-conotoxin, alpha-bungarotoxin and their pharmaceutically acceptable salts and optical isomers, and high-concentrations of bilirubin. In one embodiment, hexamethonium was injected locally. Alpha7-nicotinic acetylcholine receptor (a7-nAChRs) antagonists may be particularly suitable for the application such tetrodotoxin, nitro-L-arginine, and guanethidine.

Agonists of muscarinic acetylcholine receptors, also found on sympathetic nerves, are responsible for inhibitory post-synaptic potentials (IPSPs) and slow excitatory (EPSPs) under certain conditions such as atropine or scopolamine or other drugs that are inhibitory such as γ-aminobutyric acid GABA or GABA agonists (e.g. GABA_{B} agonists), or baclofen. Alternatively, some antagonists of muscarinic receptors may also be selected to reduce nerve transmission, such as pirenzepine. Aromatic amino acid hydroxylase inhibitors, that inhibit tyrosine hydroxlase activity including halogenated phenylalanines, 3-alkly methyltyrosines, 3-substituted alpha-methyltyrosines, and 3-alkyl-methyltyrosines or antibodies to tyrosine hydroxylase, or antihypertensive drugs that modulate TH activity, such as anti-DBH, hydralazine, may be employed. Drugs that degrade catecholamines or drugs that inhibit the biosynthesis of norepinephrine as anti-dopamine beta-hydroxylase (anti-DBH) or DBH inhibitors such as nepicastat, or hydralazine may be employed. MAO enzymes' primary role is the metabolism of exogenous amines, control of neurotransmitter levels and intracellular amine stores and in the catabolismof neurotransmitters in the periphery. MAO-A preferentially oxidizes serotonin and norepinephrine, whereas MAO-B oxidizes phenylethylamine (PEA), with dopamine and tyramine being substrates for both isoenzymes.

There are several other classes of drugs of interest that modulate norepinephrine release. Several agents that modulate norepinephrine release indirectly include Autoinhibitory Alpha-2 adrenoreceptor agonists, mimicking the action of norepinephrine, are located primarily on the presynaptic postganglionic nerve ending and inhibit the further release of norepinephrine from the neuron and may reduce the neuronal supersensitivity. These agents include dexmedetomidine, oxymetazoline, rilmenidine, moxonidine, agmatine and clonidine and non-selective phenoxybenzamines. The latter agents also act on the imidazoline (1) receptors. Non-selective alpha blockers, acting on alpha-2 receptors include phentolinamine, an irreversible phenoxybenzamine. 2) Nucleoside transport inhibitors. For example, draflazine, which increases adenosine concentrations in the synaptic cleft which in turn inhibit norepinephrine release through stimulation of pre-synaptic receptors, 3) Autoinhibitory H3 histamine receptor agonists such as (R)-alpha-methylhistamine, which inhibit the co-release of norepinephrine and histamine. 4) Serotonin (5-HT) acting on an autoinhibitory receptor, has also been demonstrated to inhibit norepinephrine release, 5) Presynaptic imidazoline receptor agonists which inhibit the release of norepinephrine including antazoline, cirazoline, idazoxan, 6) Guanidines such a guanidine chloride and derivatives, aganodine, arginine, and saxitoxin have been demonstrated to inhibit norepinephrine release from sympathetic nerves, 7) non-depolarizing neuromuscular blocking drugs that also modulate sympathetic efferent and afferent nerves including Cistracurium besilate (Nimbex), one of the isomers of atracurium, 8) hormones such as estrogen (beta-estradiol) which reduce NGF protein and TH protein content and reduced sympathetic neuron survival, and 9) sustained release of tyrosine hydroxylase inhibitor metirosine (alpha-methyl-p-tyrosine or AMPT) that has been demonstrated to reduce the release of norepinephrine and epinephrine, 10) In addition, angiotensin type II receptor blockers such as losartan may inhibit sympathetic nerve activity 12) In another embodiment, blockers of the p75 receptors and tropomyosin-related receptor tyrosine kinases (Trk), such as TrkA, reduce adrenergic output and neuronal survival. Blocking the activation of TrkA by NGF prevents the potentiation of an excitatory noradrenergic transmission at the neuron-myocyte synapse. Similarly, stimulating the p75 neurotrophic receptor promotes inhibitory acetylcholine release. 13) Adenosine and adenosine agonists, activates K+ and Cl− conductances, limits synaptically evoked depolarization and Ca2+ influx, directly protecting neurons against Ca2+ mediated overload but at high concentration cause apoptotic cell death.

Drugs that improve the reuptake of norepinephrine from the synaptic cleft directly or indirectly include perindopril, candesartan, and valsartan, or through modulation of dynamin-mediated endocytosis and vesicle cycling. Another approach is to reduce the activity of the pre-ganglionic neurons. Alfuzosin, (10-40 mM) the alpha-1 receptor antagonist, reduces sympathetic pre-ganglionic sympathetic nerve activity and thus reduces the post-ganglionic nerve firing and norepinephrine spillover. In yet another embodiment, p75 neurotrophin receptor (p75NTR) agonists, such as through proNGF, NGF, LIF, IL-6, IL-1, TNF-alpha, BDNF or proBDNF may denervate the sympathetic ganglia and block sympathetic sprouting to the DRG.

In another embodiment, it is desirable to prevent the activation of visceral afferent nociceptive C fibers that either travel with the sympathetic or parasympathetic nervous system, so as to block or reduce the transmission of pain. Since many of these fibers travel with the sympathetic nervous system through the sympathetic chain, therapies designed to modulate this class of neurons alone or in combination with sympathetic efferent fibers is desirable. NGF is a survival factor for both developing afferent and sympathetic efferent nerves, and has recently been demonstrated to play an important role in the generation and perpetuation of neuropathic, inflammatory and ischemic pain and hyperalgesia across the afferent (somatic/visceral) and sympathetic efferent neurotransmission. In one embodiment, an agent that blocks or antagonizes NGF or blocks its binding to TrkA is administered. Drugs that reduce the survival and/or axonal outgrowth after injury, such as through sequestration or reduction in nerve growth factor (NGF) levels, may be desirable. These include neurotrophic Tyrosine kinase receptor A (TrkA or NTRK1) antagonists that sequester NGF via the TrkA domain 5, antibodies to TrkA or NGF, such as local anesthetics.

In another embodiment, a patient may be prescribed reserpine and/or one, two, or more therapeutic agents as disclosed for example herein orally, intravenously, subcutaneously, intramuscularly, transdermally, or through another route of administration for one to three days prior to the procedure in order to lower their systemic norepinephrine levels prior to the procedure. In another embodiment, the patient continues to take reserpine or other therapeutic agent(s) for a specified time such as 30 to 60 days after the procedure.

In another embodiment, delivery of leukemia inhibitory factor (LIF), interleukin-6 (IL-6) or ciliary neurotrophic factor (CNTF) will trigger in a switch from adrenergic nerves to a cholinergic phenotype, reducing the release of norepinephrine. In another embodiment, inhibitors of LIF, IL-6 and CNTF may be delivered to reduce the sympathetic nerve sprouting to form connections with the dorsal root ganglion. In another embodiment, blocking the sodium Navv1.6 channel reduces pain, sensory neuron excitability and sympathetic sprouting. In another embodiment, beta-3 adrenoreceptors antagonists are delivered to the region containing the DRG to modulate the sympathetic post-ganglionic activity.

In indications in which acute and subacute control over local or systemic neurotransmitter levels is not necessary or desirable, alternative neuromodulatory agents can be employed to modulate the nervous system, particularly the sympathetic nervous system. Drugs that hyperpolarize cells to temporarily cause hyperexcitability through the increased dumping of neurotransmitter only to later cause the nerve to degenerate are of particular interest. These include tricyclic anti-depressants and other anti-depressants, and other nonspecific agents such as those that temporarily increase the levels of neurotransmitter in the synaptic cleft before blocking and reducing neurotransmitter levels. These agents can be used in combination with anesthetics to reduce or eliminate the norepinephrine release. Secondarily, agents that result in excitotoxic cell death, in which neurons are damaged or killed by excessive stimulation are described.

In some embodiments, the patient's own blood is mixed with the gel to form an inhibitory clot. This can be performed first with a sample of the patient's blood ex vivo and then the blood is injected back into the target tissue. Alternatively, the polymer or peptide can be injected into the target tissue where it crosslinks with local fibrin (e.g. PolySTAT). In another embodiment, a fibrin gel, prepared from fibrinogen and thrombin is prepared.

Alcohol and phenol (carbolic acid, monohydroxybenzene) are both commonly used neurolytic agents. Alcohol causes an immediate progressive burning paresthesia that lasts several hours but a wide range of ethanol concentrations are effective at destroying nerves through extraction of cholesterol and phospholipids and subsequent sclerosis. Concentrations above 50% are well established to result in neurolysis, such as about 75%, 80%, 99% or 100%. One-hundred percent ethanol has been demonstrated to completely destroy the cell bodies and axons of sympathetic, sensory and motor neurons but come with a higher risk of adjacent neuritis. Phenol has mild anesthetic properties and causes a focal hemorrhagic necrosis and dissolves axons and Schwann cells inside the basal lamina, resulting in damage to the entire endoneurium. Regeneration in the periphery may begin in 2 weeks in preclinical studies. The drug can be injected at, for example, between 3 and 10%, more typically between 6.7% to 7% in oil or glycerol, such as Phenol-Aqua (7%) or phenol-glycerol (5%). Higher concentrations have been applied, such as about or at least about 10%, 25%, 50%, and 75%, such as between about 10-50% phenol in ethanol is desirable in some cases. Both produce severe burning pain immediately upon injection which may last about a minute. Glycerol is an anhydrous less toxic alcohol with weaker penetration, less extensive neuronal damage and faster regeneration than alcohol and phenol. lohexol (30%) may also be employed. Alternatively, sodium tetradecyl sulfate (STS), an anionic surfactant and sclerosant drug with detergent properties may be selected. In some embodiments, the ethanol is loaded into the hydrogel at a concentration of 20% or higher, more preferably 50% of higher in order to have a neurolytic effect. In yet other embodiments, the ethanol is co-delivered with a neuromodulatory drug, such as amiodarone, to co-deliver a neuromodulator or nerve blocking agent with a neurolytic agent. The ethanol may improve the solubility and deliverability of the drug and improve the release rate.

Dissolving neurons or inducing changes such that the nerves are resorbed from the site of injury. Of interest is the ability to delivery to dissolve the nerves at the site of injection. In addition to traditional neurolytic agents, this can be accomplished by the application of nonspecific cytolytic agents such as surfactants or acids such as deoxycholic acid (KYBELLAR) either in combination with or within the in situ forming hydrogel. For example, 20 mg of synthetic deoxycholic acid can be delivered in an ethanol-loaded PEG hydrogel.

By incorporating these readily available neurotoxic agents into a formulation that will slow or control their spread, adverse events and complications arising from their use can be limited. In one embodiment, ethanol is incorporated in the gel as a solvent for the neuromodulatory agent that is delivered. In another embodiment, there is no neuromodulatory active agent and ethanol alone provides the neurolytic effect but its spread is controlled by its containment within a formulation. In one embodiment, after delivery, the rapid tissue absorption of ethanol into the surrounding hydrophobic neurons and adipocytes causes the liquid formulation to gel.

Norepinephrine reuptake inhibitors (NRIs) and less specific norepinephrine serotonin reuptake inhibitors (SNRIs) (and selective serotonin/5-hydroxytryptamine reuptake inhibitors (SSRIs) and dopamine reuptake inhibitors) block the reuptake of norepinephrine at the synaptic cleft thereby increasing and sustaining the action of norepinephrine at the nerve terminal in the heart and other tissues. Norepinephrine uptake transporters (NET) includes Uptake 1, present in the neurons and lung pulmonary endothelial cells and uptake 2 transporter, present in the myocardium. Reuptake inhibitors include guanethidine, 1-methyl-4-phenyl-pyridinium ion (MPP+) and Oxidopamine or 6-hydroxydopamine (6-OHDA), alpha-methyldopa, bretylium tosylate, guanacline, bethanidine and debrisoquine, desipramine, nisoxetine, ritanserin, setoperone, volinanserin, duloxetine, citalopram, fluvoxamine, zimeldine, sibutramine, Levomilnacipran, debrisoquine, lobeline and amezinium. Dopamine reuptake inhibitors include GBR-12909 and amfonelic acid. Many of these agents also function as MAO inhibitors to prevent norepinephrine deamination and some as a VMAT agonist. Although not a reuptake inhibitor, alkaloid cocaine interferes with Uptake-Guanethidine (1-2 mg/ml) is particularly interesting in some embodiments because it can both increase the norepinephrine in the synaptic cleft (transient sympathomimetic) initially through NET1 activity but also acting as a monoamine depleting agent, and blocks adrenergic transmission. High or sustained doses lead to neuronal cell death in both efferent and afferent nerves, such as capsaicin-sensitive primary sensory nerves. Preferably, these agents are delivered to nerve terminal or peripheral synapse of the post-ganglionic sympathetic nerve in the heart, lung, or tissue innervated by post-ganglionic sympathetic efferent nerves. At high concentrations, these agents result in immunotoxic NK- and mononuclear-cell mediated death as can be seen by degeneration of sympathetic ganglia in the sympathetic chain.

Anti-depressants. In another embodiment, the neuromodulatory agent is an anti-depressant such as bupropion, doxepin, desipramine, clomipramine, imipramine, nortriptyline, amitriptyline, protriptyline, trimipramine, tianeptine, fluoxetine, fluvoxamine, paroxetine, sertraline, phenelzine, tranylcypromine, amoxapine, maprotiline, trazodone, venlafaxine, mirtazapine, their pharmaceutically active salts and/or their optical isomers. In a very preferred embodiment, the anti-depressant is either bupropion or a pharmaceutically acceptable salt thereof, or nortriptyline or a pharmaceutically acceptable salt thereof. Bupropion, desipramine and imipramine are also ganglionic blocking agents (nicotinic) and at higher doses is toxic to afferent and efferent nerves.

Microtubule disrupting agents or cytoskeletal drugs that interact with actin or tubulin may also be used to denervate neurons such as phalloidin, cytochalaisin D, Latrunculin, colchicine (1 and 10 microM), demecolcine, jasplakinolide, nocodazole, paclitaxel (taxol), vincristine, vinblastine. Other potential approaches include inhibition of phophoinositide 3-kinase (PI3K), serine-threonine protein kinase B (Akt), extracellular signal-regulated kinase (ERK) pathway, the P38 mitogen activated protein kinase pathway (MAPK).

Cholesterol oxides (PMID 9566506) cause rapid cell sympathetic ganglia cell death in vitro at concentration of 4 μg/ml (10 μM) within 36 hours. The most potent of these 25-OH-cholesterol has demonstrated neurotoxicity across a range of cell types.

MAO-A and COMPT inhibitors, including tyramine, clorgyline, paragyline and 3,5-dinitrocatechol, Ro 41-1049, selegiline, tranylcypromine may result in excitatory chemical sympathectomy if delivered in high enough levels.

Immunosympathectomy can be achieved with Anti-Nerve growth Factor (anti-NGF, Tanezumab, Fulranumab), auto-immune sympathectomy with Anti-Dopamine Beta Hydroxylase (DHIT), DBH or Anti-acetylcholinesterase (Anti-AChE, immunotoxin sympathectomy with OX7-SAP, 192-SAP IgG, DBH-SAP or DHIT. Toxins such as botulinum toxin (BOTOX, DYSPORT type A through G, such as described, for example, in U.S. Pat. No. 6,743,424 to Donovan, which is hereby incorporated by reference in its entirety), tetrodotoxin, neosaxitoxin, may also be effective.

Efferent nerve degeneration. Botox can be advantageous in some embodiments for delivery to the sympathetic chain because it preferentially targets the pre-ganglionic sympathetic efferent nerves by blocking the release of acetylcholine. Blocking the sympathetic pre-ganglionic stimulation of post-ganglionic nerves has been demonstrated to dramatically reduce norepinephrine release from the post-ganglionic nerves. Botox injections typically last between 3 to 6 months, providing a sustained reduction in efferent nerve activity during this time. Furthermore, denervation of pre-ganglionic fibers, while leaving the post-ganglionic fibers intact results in reduced denervation supersensitivity (upregulation of beta-adrenergic receptors in the heart). As with all drugs, ‘off-target’ effects on post-ganglionic and afferent nerves are likely, although the agent will likely have a less powerful effect on these nerves.

Afferent nerve degeneration. High concentrations of capsaicin result in a stimulatory denervation of afferent nerves with small diameter soma (dark B-type)>intermediate diameter soma (light-A type)>type C fibers>thinly myelinated Aδ fibers and therefore may be used to provide short-term degeneration of sensory somatic or visceral fibers. Alternatively, resiniferatoxin (RTX) underway for trials for intrathecal administration for intractable pain, may also be suitable for applications in the paravertebral gutter as it is a highly selective agonist of the TRPV1 receptor and can selectively ablate afferent neurons. RTX is a potent agonist of the vanilloid receptor-1 (TRPV1), a pain sensing ion channel expressed in dorsal root ganglion A-delta and C fibers neurons, confers specificity to largely sensory neurons and exhibits cytotoxicity through calcium-mediated excitotoxicity. Since RTX is expressed not only at the ganglia and the nerve terminal but along the axons of primary afferents (and interneurons), local delivery to RTX to the afferents coursing along and through the sympathetic chain is desirable. RTX is thought to apoptose neurons selectively through an overload in calcium influx. The enhanced cardiac sympathetic afferent reflex (CSAR) may contribute to exaggerated sympathoexcitation in chronic heart failure. In one study, 50 μg/ml RTX (10% ethanol in 10% Tween 80/isotonic saline) was painted twice on the right and left ventricles with a brush to achieve complete ablation of TRPV1 expressing cardiac nerve endings for over 10 weeks. The RTX does not appear to impact parasympathetic or sympathetic efferent innervation of the heart but instead not only blocks the TRPV1 receptors but delete the TRPV1 receptors on cardiac afferents and damages the TRPV1 expressing afferent nerve endings. Thus, RTX can prevent the exaggerated renal and cardiac sympathetic efferent nerve activity that the cardiac visceral afferents play in potentiating cardiac disease, potentially through central and extraspinal reflexes.

The ability to deliver RTX, e.g., in a gel in a sustained and controlled way can be desirable. In particular, avoiding the uncontrolled spread of the toxin to off-target tissues is desirable. For example, in the case of cardiac applications of RTX, limiting the delivery of the drug to the cardiac tissue either through direct application or indirectly to the nerves innervating the cardiac tissue, can be desirable. In one embodiment, the application of RTX can be adapted for delivery in a hydrogel for delivery to the epicardium either transvascularly such as through the coronary arteries, transatrially or transventricularly. In another embodiment, the application of RTX can be adapted for delivery into and around (but not limited to) the stellate ganglion, the paravertebral gutter including the stellate ganglion to the lower thoracic ganglia, and the epidural space including the epidural root containing the dorsal root ganglion. By targeting afferent axons closer to their cell bodies or targeting the cell bodies themselves, as in the case with a paravertebral and intrathecal injection, respectively, degeneration can be more complete and regeneration is more limited. In addition, by targeting the dorsal root ganglia themselves, a more permanent afferent block can be achieved through the administration of RTX to the dorsal root. Finally, by delivering the RTX in a gel, the reinnervation of the tissue may be physically hindered by the physical barrier presented by the hydrogel, obviating the need for repeat injections. This approach can be modified to treat renal and cardiac dysfunction and thus reduce cardiac hypertrophy, apoptosis, fibrosis as well as reduce High LVEDP and lung edema. RTX could also be utilized in a gel for the treatment of chronic intractable cancer pain, or delivered intrathecally for a longer period of time with a catheter.

In one embodiment, the RTX-loaded hydrogel is delivered into the paravertebral gutter and in another embodiment it is delivered into the intervertebral foramen containing the dorsal root ganglion. The cervical, thoracic, or lumbar levels targeted are derived from the appropriate levels required to treat the specific disease and are driven by the innervation of a given target organ or target dermatome. In one embodiment, the final loading level of the hydrogel is between about 0.1 and 1 mg/ml, more preferably 0.25 mg/ml. The release of the RTX into the surrounding neural tissue can occur between 1 and 14 days post-injection of the neurolytic-hydrogel. For example, in one formulation, the RTX powder is mixed with the PEG powder and resuspended the SpaceOAR hydrogel kit to form a final concentration of 0.5 mg/ml in a 5 ml syringe.other formulations with RTX.

Other vanilloids, such as capsaicin, may be used, but can require high concentrations of drug in order to have a lasting effect. In one embodiment, the application can include between 1.0% and 10% loading in order to achieve efficacy.

Some anesthetics have relatively selectivity for C fibers and can potentially be administered at a concentration that ablates afferent fibers but only blocks other fibers types. In some embodiments, the delivery of excitotoxins such as kainic acid (0.5 nmol/ul), kainate/kanamycin, or N-methyl-D-aspartic acid (NMDA, 6.8 nmol/ul) and NMDA subtypes, okadaic acid, GM1 ganglioside, quisqualate or α-amino-3-hydroxy-4-isoxazoleproprionic acid (0.54 nmol/ul) can be applied that have been demonstrated preclinically to target only afferent, not efferent fibers. These agents result in considerable loss of cell bodies but spare axons and appear to be selective for the denervation of vagal afferent neurons but only stimulatory to the afferent dorsal root fibers.

Drugs-gene therapy. Alternatively, a gene therapy based approach to silencing nerves can be used to effectively halt neural activity of a pathway. For example, DNA, RNA, RNAi, and/or siRNA can be delivered to send a neuron into a pro-apoptotic pathway, to deliver a light-sensitive protein so that light can halt neurotransmission in a cell, such as described in U.S. Pub. No. 2013/0225664 to Horsager et al., which is hereby incorporated by reference in its entirety. Neurotrophic factors (e.g. GDNF, BDNF, or NGF) or neurotrophic factor receptors (e.g. TrkA or p75 low affinity NGF receptors) can be knocked out or deleted. Alternately, the phenotype of neurons can be altered such as from noradrenergic to cholinergic or neurotransmitter synthesis can be up or down regulated through changes in transcription and translation factors. Examples of proteins whose expression can be directly or indirectly up- or down-regulated include leukemia inhibitor factor, phenylethanolamine-N-methyl transferase (PNMT), tyrosine hydroxylase (TH) or DBH.

Modulating supersensitivity may be desirable specifically pre- or post-junctional supersensitivity, in which the responsiveness of cells is characterized by a leftward shift of concentration-response curves for agonists.

Suicide axoplasmic transport (retrograde). In yet another embodiment, a neuromodulatory agent can be delivered at the distal nerve terminals and retrogradely transported to the ganglia to modulate the nerve. In this manner, nerves innervating a target tissue or organ can be selectively denervated, such as, for example, with Adriamycin or Epirubicin. In one embodiment, 0.05 to 1 mg of Adriamycin (doxorubicin) can be delivered to the heart, lung, and great vessels of the thorax to selectively destroy non-motor afferent and efferent sympathetic and vagal fibers, providing an avenue to deliver the cytotoxic drugs to nerves away from the intrathecal space. These tissues include the pericardial sac, the epicardial fat pads, the nerves traveling along or across the coronary arteries, coronary veins, coronary sinus, the nerves traveling around the pulmonary arteries/veins, the pulmonary artery trunk, the aorta, the bronchial arteries/veins or lung root/hilum. These agents may be delivered through a transvascular or percutaneous or other minimally invasive approach. If performed carefully, high local concentrations of Adriamycin can be delivered to the sympathetic chain with careful avoidance of the intrathecal space, to achieve post-ganglionic sympathectomy. This cell death can also be achieved with other retrogradely transported agents such as Ricinus communis agglutinins and highly toxic lectins for example. Alternatively, neurotoxic or neurolytic drugs can be conjugated to retrogradely transported peptides or proteins, such as wheat germ agglutinin (WGA), dextran, horse radish peroxidase (HRP) for rapid axonal transport to the perikaryon from a nerve terminal or crushed/transected nerve. In another embodiment, the retrograde transport can be used to deliver drugs that support neuronal survival and/or regeneration. In another embodiment, viruses than are known to be transported retrogradely and transsynpatically can be used to deliver neuroprotective agents from the periphery directly to the target cells in the central nervous system, such as the hypothalamus. In one embodiment, the retrograde transsynaptic tracer pseudorabies virus (PRV) coated nanoparticle loaded with NGF can be delivered to the paravertebral gutter for delivery both locally and to central nervous system. For example, agents can be delivered into the paravertebral gutter that are then taken up by pre-ganglionic neurons and transported transynaptically to the locus coeruleus for the treatment of Parkinson's disease. These agents may also provide for improved local survival of neurons within the sympathetic chain, as these patient's also suffer from loss of cardiopulmonary sympathetic nerves.

Double crush or synergistic. In some embodiments, nerves may receive a “double crush” in which two, three, or more therapeutic agents or factors, in series or in parallel, lead to effective neuroablation through, in part, a synergistic effect. The first factor may be the presence of an existing precondition such as neuropathy or diabetes prior to receiving the second ‘crush’, the neurolytic agent. In another embodiment, the first crush may be a systemically administered agent, (whether delivered, for example, orally, intravenously, intraarterially, intraperitoneally or as an inhaled agent) that lowers the threshold for neurotoxicity before a local agent is delivered to ablate the nerves in the region. In one embodiment, reserpine is administered systemically, and then a neurolytic agent, such as lidocaine, is administered locally, as described, for example, in U.S. Pat. No. 4,029,793 to Adams et al. or U.S. Pat. No. 7,928,141 to Li, both of which are incorporated by reference in their entireties. In another embodiment, an anesthetic agent is delivered first to block the nerves and reduce the pain, and then subsequently a neurolytic agent is delivered that acts synergistically with the anesthetic agent to locally denervate the neurons. In another embodiment, a local agent is delivered and coupled with a mechanical or thermal signal to cause more complete neuronal cell death, such as a neurolytic agent combined with high-frequency ultrasound or radiofrequency ablation. In another embodiment, the synergistic effect may allow for a reduction in the dose or concentration of one or both agents, thereby reducing systemic toxicity.

In another embodiment, other drugs with known neuromodulatory effects, many with transient or acute excitotoxic effects, include, for example, glutamate, glutamine, polyglutamine, isoniazid, crotoxin, taipoxin, phenylephrine, tryptamine or 5-hydroxytryptamine, chlorpromazine, clozapine, doxorubicin (TRPV1), NMDA, MPTP, chlorpromazine and other phenothiazines, ampicillin, N-(2-Chloroethyl)-N-ethyl-2-bromobenzylamine (DSP-4), lanthanides, yohimbine, nicotine and lobeline and amphetamine (mixed agonist-antagonists), nicitinamide and nicotinic acid and derivatives, lectins, trimethyltin (TMT), NSAIDs such as indomethacin, nitrosoureas such as streptozotocin, streptomycin, gentamycin, bleomycin, 6-hydroxydopamine (100 mg/kg sc), kainite, quinolinic acid, phenytoin, bupropion, thalidomide, quinolinate, fluoroquinolone antibiotics such as moxifloxacin, levofloxacin, and ciprofloxacin, varatum alkaloids such as proveratrine or veratridine, vinca alkaloids such as vincristine, bortezomib (glove and stocking peripheral neuropathy), rotenone (pesticide), yessotoxin (increase in cytosolic calcium), brevetoxin (L-glut and L-asparate), the fluoroquinolones including ciprofloxacin, gatifloxacin, gemifloxacin, and levofloxacin; myelin fludarabine, methotrexate, vinblastine sulfate (0.4 mg/kg sc,) vincristine, cisplatin, oxaliplatin, ormaplatin, gentamycin, gemcitabine, sorafenib, angiotensin II agonists, saralasin, bleomycin, taxol/paclitaxel, L-arginine, phenytoin, caspace, caffeine, captopril, paclitaxel which induce ceramide synthesis include chemotherapeutic agents, gamma interferon, matrix metalloproteinases, and anandamide. Corticosteroids such as prednisone, methylprednisolone, triamcinolone diacetate, triamcinolone acetonide, or betamethasone can also be utilized. Drugs which block GABA-ergic transmission of sympathetic ganglia such as bicuculline and metrazol, VMAT-2 agonist methylphenidate, amphetamine, the powerful toxic lectin ricin, ergotoxine or ergotamine or ergotoxine derivatives (ergocristine, ergocornine, ergocryptine, methysergide) paralyze the sympathetic nervous system or cabergoline, pergoline or lisuride, gambierol, pyrethroids, ivabradine, mibefradil, nicorandil, trimetazidine quinapril, losartan, droperidol, tramadol, labetalol, spiperone, picrotoxin, butyl aminobenzoate, HA H3 receptor antagonist thioperamide, opipramol, pentazocine, lacidipine, 3-hydroxy-3-methylglutaryl coenzyme A (HMG-CoA) reductase inhibitors including mevastatin and lovastatin; rimcazole, panamesine, rimcazole, metaphit, tizanidine, apraclonidine, oxaprotiline, and spermine are some examples. Alternatively, sustained release of fast anterograde transport blockers has been implicated in acrylamide and gamma-diketone-mediated, and 2,5-hexanedione (2,5-HD) nerve degeneration.

Other non-pharmacologic chemical agents include salicylic acid (10% in ethanol), menthol, isotonic dextrose, hypotonic saline (e.g., half-normal saline or less, or dextrose in water), hypertonic saline (10%, 1.7M, 100 mg/ml) severe pain) or hyperbaric solution (5-8% glucose) which may be effective against C-fibers. Yet other agents include liquid nitrogen and hydrogen peroxide, octanoic acid, methanol, D-limonene, kainic acid, domoic acid, diethyl ether, and L-2-chloropropionic acid.

Similarly, many of these agents may initially increase neurotransmitter levels at the synaptic cleft but rapidly halt neurotransmission resulting in catecholamine depletion at lower doses and then cause degeneration at higher doses possibly through an immune related mechanism or cause an initial upregulation in neurotransmission followed by neurodegeneration. Further examples of therapeutic agents that can be utilized with systems and methods as disclosed herein can be found, for example, in U.S. Pat. No. 6,932,971 to Bachmann and U.S. Pub. No. 2006/0280797 to Shoichet et al., U.S. Pub. No. 2015/0132409, 2015/0202220, U.S. Pat. Nos. 8,975,233, 9,056,184 to Stein et al., U.S. Pub. No. 2013/0252932 and U.S. Pat. No. 9,011,879 to Seward, all of which are incorporated by reference herein in their entireties.

In another embodiment, the hydrogels can be modified with peptides that are growth-inhibitory to neurons (as opposed to most modifications to improve ingrowth).

In some embodiments, it can be desirable to protect a first population of nerves adjacent or in proximity to another second population of nerves that are receiving a neuromodulatory therapy or undergoing some other treatment. In one embodiment, the soma, ganglia, plexi, that require neuroprotection can be surrounded with a neuroprotective biocompatible ‘blank’ gel in which the neuroprotection is provided by the compliance of the gel with the tissue and the mechanical barrier of drug diffusion through the gel or formulation. In this manner, neurolytic or neurotoxic agents can be delivered alone or in combination with a gel the adjacent tissue while protecting other critical structures or tissues. The hydrogel may also protect other vital structures from chemical, thermal, or mechanical damage. Another embodiment involves the delivery of a neuroprotective agent or agent that antagonizes or attenuates the effects of the neurolytic agent within the gel. Like combinations with a neurolytic agent, delivering a gel with a neuroprotective agent that is retained in the gel and has limited spread to the regions in which the neurolytic agent is delivered can be desirable. For example, a neuroprotective agent can be applied to the nerves that it is desired to spare directly or in a sustained release formulation, such as a hydrogel. This procedure can be performed percutaneously or endovascularly prior to the injection of a neurolytic agent either alone or in a hydrogel. This may be performed through the same delivery effector such as a needle, such as through a double lumen with two exit ports facing opposite each other or another angled orientation with respect to the sidewall of the catheter. In some embodiments, delivery of the hydrogels may be performed through the same lumen but the gels are injected serially with the blank or neuroprotective-gel delivered first followed by administration of the neurolytic gel or vice versa. In one embodiment, the delivery of the neuroprotective agent/gel can be delivered in one location through a needle or lumen and then the needle or lumen can be rotated and oriented 180° (such as rostral followed by caudal) to deliver the neurolytic agent/gel to a second location to the nerves it is desired to ablate. For example, it may be desirable in some embodiments to ablate or neuromodulate only the T1 sympathetic ganglia and below. Because 80% of the time the T1 ganglia is fused completely or partially with the inferior cervical ganglion, it may be desirable to deliver a neuroprotectant gel or blank gel at the 1st rib to the inferior cervical ganglia through an ultrasound- or CT- or other image-guided approach. This guidance, for example, may be done utilizing one of the anterior approaches to access the stellate ganglion, as performed with stellate ganglion block. After this, either through the same lumen or through a catheter extended within or over the existing lumen, a neurolytic gel may be administered to the lower half or third of the stellate ganglion, the T1 sympathetic ganglion. In doing so, avoidance of Horner's syndrome may be possible. Specifically, the lumen can be directed upward toward the inferior cervical ganglion at the upper margin of the 1st rib to deliver a blank hydrogel. The lumen can then be rotated caudally to deliver a neurolytic-hydrogel to T1 to T4, as desired. Alternatively, injections of neuroprotectant gel or ‘blank’ gel can be made into the relevant intercostal spaces to prevent the intercostal nerves against intercostal neuritis, or into the dorsal or ventral roots, or into the intervertebral space or the intrathecal space to prevent spread to the roots or spinal cord, respectively.

In other embodiments to permit neuroprotection, one group of neurons can be protected via the distal delivery of agents at their axons or nerve terminals. The neuroprotective agent delivered there may block or reduce the activity of the specific population of nerves that it is desired to protect. In this embodiment, the neuroprotective agent and/or gel may be delivered distally to the targeted neuroablation zone along the axons or nerve terminals to have neuroprotective effects on the soma and axons directly within the neuroablation zone. In one embodiment, the agent is an anesthetic and protects the neuron from excitotoxic cell death. Alternatively, the agent may act as a specific antagonist to the mechanism of the neurolytic agent that is delivered. The neuroprotective agent may, for example, exert an effect for 24-48 hours, the same duration of the activity of the neurolytic agent at the injection site. In order to achieve this, injections of the neuroprotective agent are made into the organ capsule (such as the pericardial sac, pleura, or peritoneum, for example, or into the vasculature suppling the organ or extravascularly to the nerve bundles coursing into the organ. This neuroprotection can also be provided to populations of neurons undergoing mechanical axotomy.

As discussed herein, gels and specifically hydrogels can be configured to protect nerves, promote survival, reduce aberrant nerve firing, and promote regeneration, as needed to nerves. In one embodiment, prior to a surgical procedure, gels can be sprayed or injected or spread onto a surgical site (through open, laparoscopic, endoscopic or other minimally invasive approaches) to protect neural tissue. In one embodiment, the gels are transparent so the underlying neural and adjacent tissue can still be visualized. In another embodiment, the gel is adherent to the nerve and has mechanical properties such that the nerve has additional mechanical reinforcement against stretching or contusion that may occur from surgical tools. In one embodiment, a prostatectomy is performed, for example, manually or in an automated fashion with a robotic surgical system such as, for example, the da Vinci robot (Intuitive Surgical; Sunnyvale, CA) and it is desirable to protect nerves. A gel can be delivered through a port of the robotic system, or separately.

In one embodiment, after performing a surgery or minimally invasive surgery on a patient, the blank or neurotherapeutic gel can be delivered to the site of the nerves. For example, in surgeries in which a nerve is retracted or distracted in order to gain access to deeper tissues or in which a nerve in inadvertently compressed or crushed or otherwise damaged thermally or mechanically during a procedure, the formulation can be delivered along the nerve bundle in order to protect the nerve from injury. In some embodiments the PEG or other formulation can assist in maintaining or repairing the structure of the nerve.

Neuron survival or neuron regenerative drugs. In still other embodiments, neuromodulatory drugs that provide a neuroprotective or neuron survival cues to the sympathetic afferent and efferent nerves are delivered directly or in a gel-based platform to provide sustained releases of pro-survival, pro-regenerative, pro-differentiation cues. For example, controlled delivery of nicotine to the sympathetic chain, increases NGF production and thus the survival encourage the survival of afferent and efferent sympathetic nerves for the treatment of neurogenic orthostatic hypotension (NOH), cardiopulmonary denervation associated with Parkinson's disease (PD), cardiopulmonary denervation associated with diabetes and pure autonomic failure (PAF). Agents may also indirectly have a beneficial effect on nerves in the CNS, such as the locus coeruleus in Parkinson's disease. Alternatively, the local sustained delivery of L-dihydroxyphenylserine (L-DOPS) can be delivered to the cardiac sympathetic nerves to generate norepinephrine since these nerves are firing at the appropriate rate.

In some proregenerative embodiments, uncrosslinked PEG can administered locally to the paravertebral gutter or other target region containing neurons. PEG be utilized as a fusogen to fuse transected axons. The PEG can help to restore the integrity of acutely transected nerve fibers in peripheral and central nerves. PEG also has a scalant effect by resealing the membranes of neurons, providing a neuroprotective effect. With this approach, PEG can be utilized to improve neuronal survival and support resprouting and regeneration. In yet another embodiment, PEG can be conjugated to graphene nanoribbons (GNR) which act as an electrical conduit and thus an electrically active scaffold upon which neurons will grow, directing their processes in the proper direction across the gap. PEG-GNRs can be mixed into the a PEG solution (e.g., 600 Daltons) at between about 0.01% to 1% wt % to achieve electrical percolation, such as about 0.5 wt %.

In some embodiments, low molecular weight, PEG, that has not been crosslinked, for example, PEG 600 has been demonstrated to promote neurite growth. In some embodiments, increasing crosslinking density between polymer chains in the case of linear polymers or increasing the branching and crosslinking of star-shaped PEGs can be utilized. Some embodiments can incorporate Silk-PEG hydrogels and/or thermosensitive heparin-poloxamer (HP) thermo-sensitive hydrogels.

PEG, particularly low molecular weight PEG, can be a growth permissive material that permits cellular invasion, revascularzation and axon regeneration, including in challenging environments such as the central nervous system (CNS). PEG is considered a fusogen, a molecular that can achieve rapid restoration of electrical continuity between transected axons. Thus, for applications that require a pro-regenerative strategy, noncrosslinked PEG, particularly low molecular weight PEG (<5,000 kDa) can be utilized.

In some embodiments, after the microsurgical scar resection, the resulting cavity is filled with polyethylene glycol (PEG 600) which was found to provide an excellent substratum for cellular invasion, revascularization, axonal regeneration and even compact remyelination in vivo.

In some embodiments, axon regeneration-supporting effects, biopolymers that can be utilized include PEG, alginate-hydrogel, and Matrigel™ (Corning Life Sciences).

### Dosing

If chemodenervation is desired, the degree of neurotoxicity may be related to the concentration or dose administered. Generally, the toxicity increases with a longer duration of exposure above the therapeutic range. For example, the highest concentration of anesthetic used in local nerve blocks is around 2% in some cases. For a neurolytic application, the local concentration of lidocaine, lignocaine, or mepivacaine delivered may be about or at least about 5%, or bupivacaine may be about or at least about 1-2%.

### Percutaneous Devices

Unmet need: In addition to the complications associated with the uncontrolled spread of agents like ethanol, the paravertebral block (PVB, anesthetic) injections themselves may be associated with an unpredictable clinical spreading pattern that can vary from time to time within a given patient. This results in a failure to achieve paravertebral anesthetic block in up to 10% of patients.

A reliable, safe approach to disrupt or block multiple contiguous levels of the paravertebral gutter can be desirable to prevent the need for guidance and repeat insertion of needle or catheter at each sympathetic chain level. In the case of percutaneous approaches, this would significantly reduce the pain and anxiety associated with the procedure and potentially procedural complications. The procedure could be achieved with a flowable therapeutic composition, such as a hydrogel in some embodiments. In some embodiments, the paravertebral gutter can be accessed endovascularly, such as via a wall of the azygous vein in some embodiments, as opposed to transcutaneous paravertebral blocks.

Therefore, in addition to the appropriate selection of neuromodulatory agent and injectable formulation, the use of an appropriate device delivery system to administer the therapy in a safe and efficacious manner can be highly advantageous in some embodiments. In some embodiments, injection of the therapy, including but not limited to a) a neuromodulatory agent or agents alone, b) neuromodulatory agents delivered in a formulation such as a hydrogel, c) excipients (such as those from the GRAS list) delivered in a formulation such as a hydrogel, d) solvents delivered in a formulation such as a hydrogel, or e) a hydrogel alone without an active agent, is delivered into the paravertebral gutter or space. The paravertebral gutter can be accessed from multiple minimally invasive approaches including both the anterior (T1) and paravertebral approach transcutaneously and an arterial or venous approach endovascularly, and it can be preferred in some cases to deliver the agent into or toward the anterior region of the paravertebral gutter to permit longitudinal spread of the agent. Alternatively, the paravertebral gutter can be directly visualized as part of a VATS or surgical procedure and the therapy delivered through the pleura into the paravertebral gutter directly or injected into and around the site after the sympathectomy. The therapy can be delivered unilaterally or bilaterally during a procedure, such as for example on the left side first, and then the right side as needed to achieve maximal therapeutic benefit or vice versa, or only the left side or the right side. Alternatively the procedures can be performed in a staged fashion. In one embodiment, patients receive a stellate ganglion block or paravertebral block with anesthetic prior to the delivery of the neurolytic-hydrogel to confirm that they are responders and to identify any challenges with the patient anatomy.

Ultrasound. In some embodiments, the injections are performed under real-time ultrasound, MRI-, CT- or fluoroscopic guidance with lower rates of complications. In one embodiment, a Philips iU22 ultrasound system (Philips Healthcare) with a high-frequency 3D 4D volume linear array transducer (VL13, 13 to 5 MHZ) is used. In another embodiment, a 180 Plus US Machine (Sonosite) with a 10-5 MHz 38 mm broadband linear array transducer (depth between 5 to 7 cm) set in ‘General’ imaging mode was used for preliminary ultrasound pinpointing followed by needle insertion. As needed, short-axis scanning can be performed with the transducer placed medially and then moved 4 to 5 cm laterally to pinpoint the respective paravertebral areas and vertebral and intervertebral level. The probe can also be rotated to scan the area longitudinally (long-axis). In the short axis view, a 17-G Tuohy needle (Epi Mini Set 17G Polymedic, Tenema) is inserted out of plane immediately down and medial to the ultrasound probe. The Tuohy needle is advanced point by point in increments, aiming at the target nervespace after feeling the click of the tip of the needle through the superior costotransverse ligament anterior to the muscle. After each advance, 0.2 ml of normal saline can be injected allow for sonographic visualization of the needle tip. Confirmation that the needle is in the paravertebral space can be performed by injecting 1 or 2 ml of saline and sonographically observing the dilation of the paravertebral space. Once the space is identified, the needle is disconnected from the normal saline syringe/barrel and after a negative blood and air aspiration test, the formulation is delivered. As required, the air aspiration test may also serve to remove the saline from the needle lumen in preparation for delivery of the therapy. In an alternative embodiment, a soft-tipped 19-G polyethylene catheter can be inserted 2-3 cm beyond the tip of the needle, the needle removed, and the therapy delivered from the catheter. In yet another embodiment, a slightly oblique ultrasound scan is performed using a curved array transducer to provide visualization of the transverse process, pleura and costotransverse ligament. After an inline approach with an 16G-18G Tuohy needle approximately 2 to 3 cm lateral from midline, 10 ml of normal saline is injected to confirm the position of the needle tip by distension of the space under ultrasound prior to delivering the therapy through the same lumen. Color Doppler imaging may be used to help determine the location of the injectate.

Blank hydrogel. In one embodiment, the target nerve region is first injected with blank hydrogel, approximately 2 to 5 ml, to surround the nerve and provide a physical barrier to the spread of neuromodulatory agent or neuromodulatory-agent loaded hydrogel, and then a second hydrogel is injected inside the first hydrogel with the first hydrogel confining the spread of the second neuroablative hydrogel. In one embodiment, the device that delivers the therapy has a valve at the distal end of the barrel, proximal to the needle that permits blank hydrogel to be delivered first followed by neurolytic agent-loaded hydrogel second. In another embodiment, the Tuohy needle is divided into two channels inside the needle with two non-contacting circular side ports on either side of the needle. In this embodiment, one exit port may be through the needle bevel and the other a side port on the opposite of the needle from the bevel. Alternatively, the needle may now permit exit of the therapy from the bevel, but from two ports on either side of the needle, each connected with the internal channels, to permit flow of blank hydrogel rostrally and neurolytic-agent loaded hydrogel caudally either serially (blank first) or together. Alternatively, an atraumatic blunt catheter can be advanced approximately 1 cm from the Tuohy needle to deliver the therapy from a side port or a two-channel catheter can be advanced approximately 1 cm from the Tuohy needle to deliver a blank hydrogel in one direction and the neurolytic agent-loaded hydrogel in the opposite direction. Alternatively, a catheter can be inserted to the paravertebral space over the trip of the needle and advanced by 1 cm before the needle is removed. In still other embodiments, a balloon or curtain or other device can be expanded from a delivery port on the needle to prevent backflow of gel to non-target levels. Alternatively, a catheter with a soft atraumatic tip that has a steering handle and locking mechanism can be advanced to preferentially deliver the therapy rostrally or caudally by preferentially directing the tip of the device rostrally or caudally. The aforementioned devices may contain another port to allow for delivery of additional anesthesia as necessary without having to move the device.

Ports for delivering therapy. Thermosensitive, shear-thinning, or other hydrogels can be injected through a single lumen or port, or multiple lumens. Cross-linked hydrogels, which typically contain two or more components, can be mixed in various settings including 1) out of the patient (pre-mixing), prior to delivery, 2) within the delivery device but still outside of the patient, 3) within the delivery device, within the patient, and 4) at the distal tip of the delivery device, inside the patient. In the case of the mixing being required near the tip of the catheter, a multi lumen catheter with a transition zone that permits turbulent flow might allow for more flexibility to mix the agent more proximally to where it is injected in some embodiments. The transition time from a solution to a gel can be controlled by relative mixing the agents and their relative proportions. In another embodiment a balloon is inflated of a compliant sheet of material is deployed to prevent spread in the opposite direction from the needle tip.

In one embodiment, a single puncture is performed at the desired level (between T2 and T8) region using a nerve stimulator guided technique. At the T3 location, the costotransverse ligament is punctured using a 21 gauge unipolar insulated needle with a conductive atraumatic short beveled tip (10 cm long, Stimuplex, B. Braun) and a nerve stimulator (initial stimulating current at 2.5 mA, 1 Hz, 9V) is advanced until an appropriate intercostal (upper thoracic) or abdominal muscle (lower thoracic) response is visualized. The needle is then further advanced anteriorly until the appropriate muscle response can be achieved with stimulation at a current less than 0.5 mA or less (1 Hz, 9 V) and the injection is performed at this location Approximately one to 15 ml of the neuromodulatory formulation, such as one to 10 ml of neuromodulatory hydrogel are delivered.

Methods related to the use of robotics for both the diagnostic and therapeutic, or combined modalities, are also described. Robotic systems can be used to deliver the therapy stereotactically through a small surgical incision (endoscopic, thorascopic etc) to the target nerve and then integrated diagnostic electrodes can be used to monitor the neural response to therapy. These robotic systems may provide advanced needle/catheter control including force sensing, temperature sensing, rotation, advancing and withdrawal, and tip curve control, balloon expansion. For example, the Stereotaxis system (Niobe Magnetic Navigation System) which has been used to treat hypertension via renal denervation might also be useful for this application. The system can include an irrigated magnetic catheter and an advanced electroanatomic mapping system allowing for advanced mapping and navigation allowing for less contrast and radiation. Alternatively, a robotically controlled steerable catheter like the one developed by Hansen Medical (Magellan or Sensei Robotic control systems) could be used to facilitate accurate navigation and delivery of the different ablative modalities (RF, Cryotherapy, neuromodulatory agents, etc.) to the target nerve.

Alternatively, an electrode catheter or electrode needle (Stimuplex Ultra, Braun) can be placed in the nerve or adjacent to the nerve so that it can be stimulated and or locally during the procedure This provides additional confirmation beyond ultrasound that the appropriate spread of the neuromodulatory agent in a hydrogel has occurred.

### Repeat Procedures.

As necessary, the agent or therapy may be delivered on successive treatment days. In one embodiment, an anesthetic is delivered to a target site(s) to confirm safety and/or efficacy and the non-reversible therapy is then delivered on the same day or within 30-days of the trial anesthetic procedure. In another embodiment, the therapy is delivered at regular intervals in order to maximize efficacy. For example, the therapy can be delivered at 0, 30, and 60 days at the same levels or adjacent levels.

instances, one, two, or more spinal levels or dermatomes are treated. In some embodiments, the target region for neuromodulation is the nerves contained within and crossing through the thoracic paravertebral space (TPVS) on their way directly or indirectly to the heart, lungs, aorta, esophagus or other organs or vessels.

Bone. Either transcutaneous or endovascular. In one embodiment, a safety device can be designed so that the therapy cannot be injected unless it is in contact with bone. In this way, the catheter tip, or the blunt needle tip are advanced and there is a mechanical probe on the tip that makes contact with the bone. When the probe tip is appropriately pushed, an internal valve inside the lumen of the catheter/needle is pushed such that the lumen lines up with the opening on the side of the catheter. In one embodiment, the opening is directed rostrally, in another embodiment it is directed caudally. In yet another embodiment, it is directed laterally. In another embodiment, communication from two channels is lined up with two openings on either side of the catheter when the probe is appropriately pushed. In one embodiment, the mechanism of actuation is spring-loaded, in another embodiment it is a button that requires less force to actuate. In another embodiment, the sensor is an impedance sensor, detecting a difference in impedance between the bone and non-bone. In another embodiment, the sensor delivers an electrical signal and measures the threshold for stimulation before this is translated into a mechanical opening. By offsetting the opening off of the tip of the catheter, delivery of the therapy is assured within the paravertebral gutter. When the probe tip is not engaged or activated, the valve or opening to deliver the agent is closed (safety valve) and it acts as a safety mechanism to prevent injection of neurolytic agent in the wrong location. In one embodiment, the device itself has a closed loop system to stimulate and record the electrical or electromyographic signals (nerve, muscle, respectively), first to confirm the correct location and then, during and after the procedure to confirm that the therapy was delivered.

The injectable nerve barrier can be placed around an otherwise intact or healthy nerve, a hyperactive or hypersensitive nerve around a damaged or crushed nerve, or around a transected nerve. The nerve barrier itself may also act to further crush or damage the nerve. The nerve barrier may contain an agent or agents that damage the nerve fibers or soma in addition to acting as a barrier. The injectable nerve barrier can also be delivered to a nerve that has an existing neuroma or in a nerve where prevention of neuroma formation is desirable.

The nerve fibers can be encapsulated by the injectable barrier, in which the nerve barrier limits and confines the neurite outgrowth from the damaged nerve but provides no path through which they can travel to form meaningful connections with target and non-target nerves or tissue (including myocytes, skeletal muscle, tendons, nerves, and other cell types). Alternatively, the nerves can simply encounter a barrier that prevents them from forming appropriate connections to distant tissue.

In the case of injections in and around the nerves in the paravertebral gutter, including the sympathetic afferent and efferent nerves, the injection of the nerve barrier in combination with a non-excitotoxic neurolytic agent could be thought of as a ‘gentle’ sympathectomy.

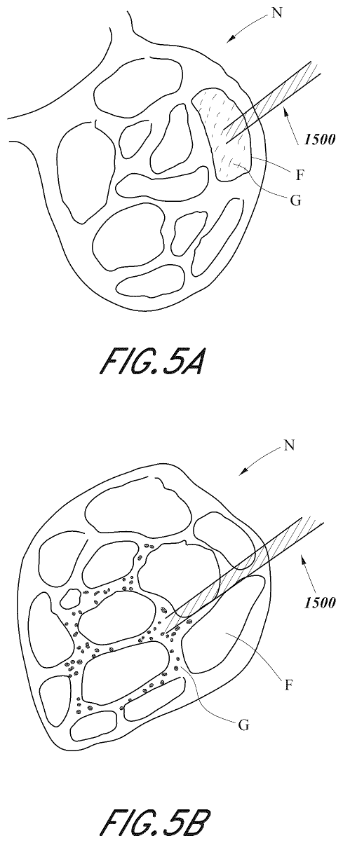

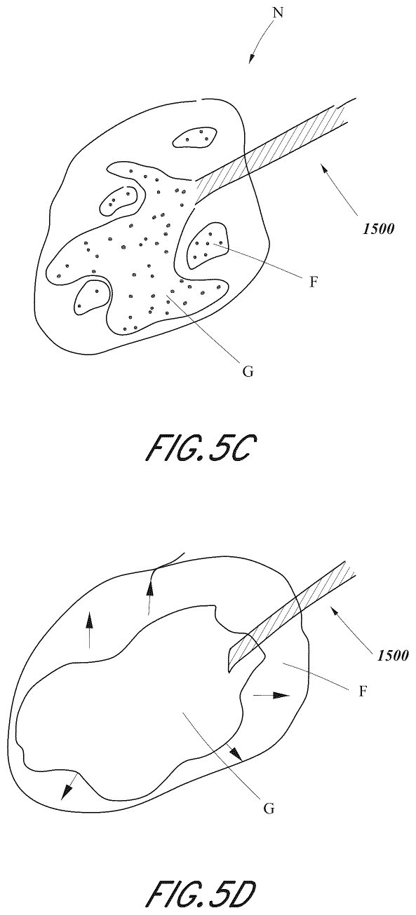

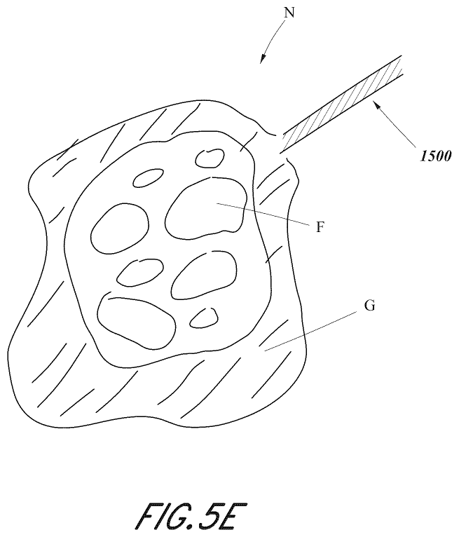

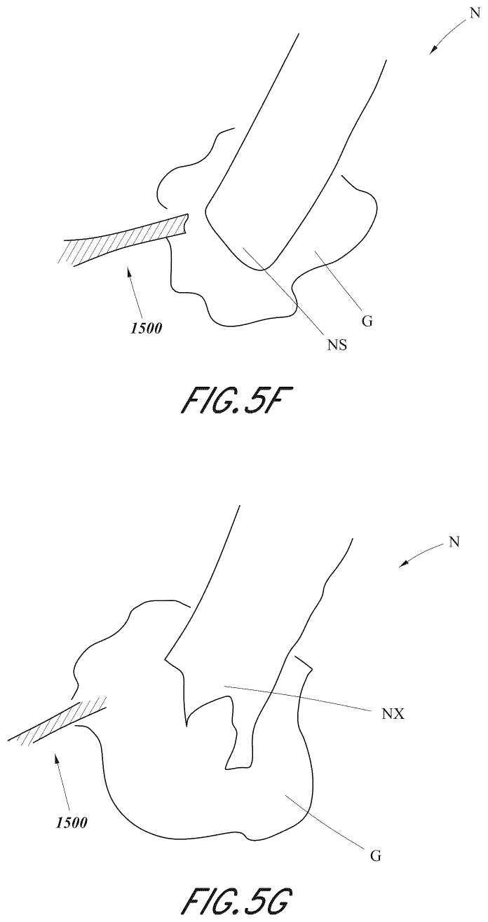

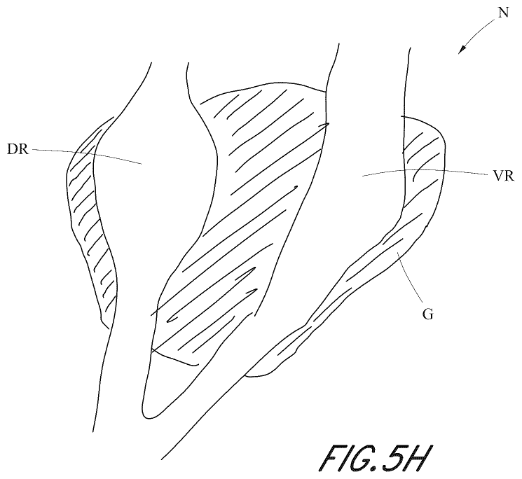

A nerve includes a number of spaced-apart fascicles (also known as nerve tracts in the central nervous system), which in turn are bundles of funiculi. A funiculus is a bundle of axons. The epineurium is the outermost layer of dense irregular connective tissue surrounding a peripheral nerve. It usually surrounds multiple nerve fascicles as well as blood vessels which supply the nerve. Smaller branches of these blood vessels penetrate into the perineurium. In addition to blood vessels which supply the nerve, lymphocytes and fibroblasts are also present and contribute to the production of collagen fibers that form the backbone of the epineurium. In the peripheral nervous system, the myelin sheath of each axon in a nerve is wrapped in a delicate protective sheath known as the endoneurium. Within the nerve, axons targeting the same anatomical location are bundled into fascicles, each surrounded by another protective sheath known as the perineurium. Any of the foregoing could be targets for injection of gels, such as hydrogels, including those disclosed elsewhere herein. FIGS. 5A-H schematically various embodiments of methods of injecting a gel into various locations within or proximate a nerve. FIG. 5A schematically illustrates injection of a gel G via injector 1500 into a target fascicle F of a nerve N (e.g., intrafascicular injection), to target one, two, or more nerve bundles of interest. FIG. 5B schematically illustrates injection of a gel G via injector 1500 intraneurally, and spreading in the region between fascicles (e.g., interfascicular injection). FIG. 5C schematically illustrates injection of a gel G via injector 1500 interfascicularly after creation of a lesion in a nerve N. The lesion can be created by ablation, dissolution, or other destruction of the nerve N, and delivered in a relatively larger volume because of the creation of interfascicular dead space as the fascicles F are reduced in volume and/or number. FIG. 5D schematically illustrates injection of a gel G via injector 1500 within a fascicle F, in which the gel G swells after injection to greater than its injected volume within the fascicle F to further reduce blood supply and crush nerve bundles. FIG. 5E schematically illustrates injection of a gel G via injector 1500 around the nerve bundle of fascicles F to prevent aberrant connections outside of the bundle, and/or to deliver therapeutic agents including drugs locally to the nerve bundle. FIGS. 5F-5G schematically illustrates injection of a gel G via injector 1500 around a transected nerve stump NS (FIG. 5F) or crushed stump NX (FIG. 5G) of a nerve N. The gel G can conform and adhere to the tissue and advantageously prevent nerve outgrowth and neuroma formation. FIG. 5H schematically illustrates injection of a gel G via injector 1500 that can be injected around neural tissue to prevent unwanted sprouting to non-target tissue (e.g., sympathetic preganglionic sprouts to the dorsal root ganglion). Illustrated are dorsal root DR and ventral root VR, although other nerves, areas near bifurcations of nerves, and the like can also be treated as disclosed herein. In some embodiments, the specific anatomy to the targeted (e.g., a target fascicle, epineurium, endoneurium, perineurium, etc.) can be precisely located under imaging guidance, such as ultrasound, CT, MRI, and the like. Injections within the fascicles typically occur at 15 psi or above and intraneural injections are typically less than 15 psi. Injections can be delivered in the intramuscular or intrafascial planes between muscles to deliver the gel around the nerve bundle.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount. Furthermore, various theories and possible mechanisms of actions are discussed herein but are not intended to be limiting.

What is claimed is:

1. A method of preventing nerve regeneration and neuroma formation in a patient, comprising:
providing a composition comprising a hydrolytically degradable bond-containing hydrogel;
identifying a target region associated with a nerve;
injecting the composition into the target region; and
contacting the nerve with the composition to form a barrier to confine neurite outgrowth from the nerve.

2. The method of claim 1, wherein the target region comprises an epidural space.

3. The method of claim 1, wherein the nerve is a damaged nerve.

4. The method of claim 1, wherein the hydrogel is adherent to the nerve for a period of 3 months or more.

5. The method of claim 1, wherein following injecting the hydrogel into the target region, the hydrogel swells to a volume greater than a volume injected to compress the nerve.

6. The method of claim 1, wherein the hydrogel is a blank hydrogel.

7. The method of claim 1, wherein the hydrogel is in situ forming.

8. The method of claim 1, wherein the hydrogel comprises one of: PEG-ester, PEG-NHS ester, or hyaluronic acid.

9. The method of claim 1, wherein the hydrogel has a compressive strength from 0 to 10 kPa or 20 kPa.

10. The method of claim 1, wherein the hydrogel a tensile strength from 10 to 15 kPa or 20 kPa.

11. The method of claim 1, wherein the composition further comprises a therapeutic agent.

12. The method of claim 11, wherein the therapeutic agent comprises resiniferatoxin (RTX).

13. A method of modulating a nerve of a patient, comprising:
providing a composition comprising a hydrolytically degradable bond-containing hydrogel;
identifying a target region associated with the nerve;
injecting the composition into the target region; and
contacting the nerve with the composition to form a barrier to confine neurite outgrowth from the nerve and prevent neuroma formation in the nerve.

14. The method of claim 13, wherein the target region comprises an epidural space.

15. The method of claim 13, wherein the nerve is a damaged nerve.

16. The method of claim 13, further comprising:
identifying the target region associated with the nerve under imaging guidance; and
injecting the composition into the target region to contact the nerve perineurally.

17. The method of claim 13, wherein following injecting the hydrogel into the target region, the hydrogel swells to a volume greater than a volume injected to compress the nerve.

18. The method of claim 13, wherein the hydrogel is a blank hydrogel.

19. The method of claim 13, wherein the hydrogel is in situ forming.

20. The method of claim 13, wherein the hydrogel comprises one of: PEG-ester, PEG-NHS ester, or hyaluronic acid.

21. The method of claim 13, wherein the hydrogel has a compressive strength from 0 to 10 kPa or 20 kPa.

22. The method of claim 13, wherein the hydrogel a tensile strength from 10 to 15 kPa or 20 kPa.

23. The method of claim 13, wherein the composition further comprises a therapeutic agent.

24. The method of claim 23, wherein the therapeutic agent comprises resiniferatoxin (RTX).
